(19) United States
(12) Patent Application Publication
(10) Pub. No.: US 2023/0329155 A1
(43) Pub. Date: Oct. 19, 2023

(54) SYSTEM AND METHOD FOR MANIPULATING AN ELECTRICAL POTENTIAL OF PLANTS AND ALTERNATIVELY FOR MANIPULATING AN ELECTRICAL CHARGE OF DISPERSED PARTICLES THAT INTERACT WITH THE PLANTS

(71) Applicant: E-DETE - PRECISION TECHNOLOGIES FOR AGRICULTURE LTD., kiryat Tiv'on (IL)

(72) Inventors: Zvi ROSENSTOCK, Rosh Pina (IL); Eylam RAN, Qiryat Tivon (IL); Rafi AGAMI, Kibutz Kfar Giladi (IL); Gal SAPIR, Kibutz Kfar Giladi (IL); Asaf BORENSTEIN, Haifa (IL)

(21) Appl. No.: 18/213,860

(22) Filed: Jun. 25, 2023

Related U.S. Application Data
(63) Continuation-in-part of application No. PCT/IL2022/051405, filed on Dec. 28, 2022.
(60) Provisional application No. 63/294,086, filed on Dec. 28, 2021.
(60) Provisional application No. 63/314,697, filed on Feb. 28, 2022.

(51) Int. Cl. A01G 7/04

(52) U.S. Cl. CPC A01G 7/04

(57) ABSTRACT

A system and method for manipulating an electrical potential of a plurality of plants, may include electrically and mechanically connecting plant electrodes to a plurality of plants, connecting the plant electrodes to a DC power source, connecting the DC power source to a growth medium via a grounding electrode; and providing DC power to the plurality of plants. The connection of the plurality of plant electrodes to the plurality of plants may include inserting at least a portion of each plant electrode into inner layers of the plant, in proximity to a lowest branching point of each plant.

### CROSS-REFERENCE TO RELATED APPLICATION

This application is a continuation-in-part (CIP) of International Patent Application no. PCT/IL2022/051405 filed Dec. 28, 2022, which claims priority from U.S. Provisional Patent Application Nos. 63/294,086 filed Dec. 28, 2021, and 63/314,697 filed Feb. 28, 2022, the contents of which are hereby fully incorporated by reference in their entirety.

### FIELD

The present subject matter relates to manipulating an electrical potential of plants and alternatively for manipulating an electrical charge of dispersed particles that interact with the plants. More particularly, the manipulation of the electrical potential of the plants and alternatively manipulation of the electrical charge of the dispersed particles that interact with the plants, is for modifying and improving growth and health of the plants and/or yield of products of the plants.

### BACKGROUND

Most plants have a natural negative electrical charge, and as a result a negative electrical potential difference between the plant and the substrate on which the plant growth, for example soil. The electrical potential of the plant is changeable due numerous so-called natural causes, for example environmental conditions, season of the year, time of day, and age of the plant, just to name a few.

Particles, for example pollen grains, insect pests, liquid (e.g., fertilizer) droplets, and the like, can have a natural electrical charge with a certain polarity: either a positive natural electrical charge, or a negative natural electrical charge, or a neutral natural electrical charge (i.e., without access electrical charge, or no polarity), and a corresponding natural electrical potential. Similarly, plants can have either a positive electrical charge, or a negative electrical charge, or a neutral electrical charge, and a corresponding electrical potential. When the polarity of the electrical charge of the particle is opposite to the polarity of the electrical charge of the plant, for example the particle is positively charged, and the plant is negatively charged, the particle is attracted to the plant. On the other hand, when the polarities of the electrical charges of the plant and the particle are similar, for example, the particle and the plant are both negatively charged, then the particle is repelled from the plant. The difference between the polarity and quantity of the electrical potential of the plant and the polarity and quantity of the electrical potential of the particle determines either the attraction, or repulsion, force, to or from the plant, that is exerted on the particle. In addition, the difference between the polarity and quantity of the electrical potential of the plant and the polarity and quantity of the electrical potential of the particle affects a trajectory and velocity of the particle motion.

### SUMMARY

Unless otherwise defined, all technical and scientific terms used herein have the same meaning as commonly understood by one of ordinary skill in the art to which this subject matter belongs. Although methods and materials similar or equivalent to those described herein can be used in the practice or testing of the present subject matter, suitable methods and materials are described below. In case of conflict, the patent specification, including definitions, will control. In addition, the materials, methods, and examples are illustrative only and not intended to be limiting.

According to one aspect of the present subject matter, there is provided a combination system for manipulating an electrical potential of a plurality of plants and manipulating an electrical charge of particles that interact with at least one plant of which electrical potential has been manipulated, the combination system comprising: at least one stationary plant system for manipulating an electrical potential of the plurality of plants; and at least one particle system for manipulating the electrical charge of particles that interact with at least one plant of which electrical potential has been manipulated.

According to one embodiment, the stationary plant system comprising: at least one power source electrically connected to a plurality of plant electrodes and at least one grounding electrode with electrically conductive cables, wherein each plant electrode is configured to electrically and mechanically connect to a plant contact point at a plant, wherein each grounding electrode is configured to mechanically and electrically connect either to another plant contact point, or to a growth medium contact point at a growth medium in which the plurality of plants grow, and that the plurality of plants are in contact with the growth medium, or a combination thereof, wherein the connection of the plurality of plant electrodes and the at least one grounding electrode facilitates electrical current flow through the plurality of plants and wherein the electrical potential of the plurality of plants, or at least one part of a plant, is affected by inducing an electrical current in the electrical circuit.

According to one embodiment, in the stationary plant system in each plant of the plurality of plants, each at least one plant electrode is configured to electrically and mechanically connect to a plant contact point. According to one embodiment, the plants are more than 1 meter apart.

According to one embodiment, in the stationary plant system, a number of the grounding electrodes is less than a number of the plant electrodes.

According to one embodiment, the plurality of plants is a plurality of trees, each comprising a trunk.

According to one embodiment, in the stationary plant system, a plant electrode is electrically and mechanically connected to a plant contact point at the trunk of the tree, substantially an edge of the trunk, above substantially a 50% trunk length.

According to one embodiment, in the stationary plant system, a plurality of plant electrodes is configured to electrically and mechanically connect to corresponding plant contact points at the trunk of the tree, substantially at a same height of the trunk within substantially 10% trunk length, and around a circumference of the trunk.

According to one embodiment, in the stationary plant system, a plurality of plant electrodes are configured to electrically and mechanically connect to one plant at different heights of the plant.

According to one embodiment, in the stationary plant system, a distance between the power source and at least one plant electrode electrically and mechanically connected to at least one plant is larger than a distance between the power source and a closest plant to the power source.

According to one embodiment, in the stationary plant system, a distance between the power source and at least one plant electrode electrically and mechanically connected to at least one plant is larger than 5 meters.

According to one embodiment, in the stationary plant system, the plant electrode is configured to electrically and mechanically connect to inner tissue of the plant. In the stationary plant system, the power source may be configured to provide direct current (DC). According to one embodiment, in the stationary plant system, the power source is configured to provide DC carrying alternating current (AC).

According to one embodiment, the combination system further comprising a control unit configured to control an operation of the combined system, to indicate or measure various parameters, and to communicate with components of the combined system.

According to one embodiment, the combination system further comprising a control unit configured to control an operation of the stationary plant system, to indicate or measure various parameters, and to communicate with components of the combined system.

According to one embodiment, the control unit is configured to monitor at least one of ambient temperature; ambient humidity; wind conditions; density of pollen in air; direction and velocity of a pollen cloud in air; voltage, current, resistance in the combination system, and any combination thereof.

According to one embodiment, in the stationary plant system, a portion of the electrically conductive cables are electrically insulated.

According to one embodiment, in the stationary plant system, the grounding electrode is an existing electrical ground.

According to one embodiment, wherein the stationary plant system further comprising an in-growth medium electrically conductive cable that is configured to electrically connect to the power source and conduct an electrical current.

According to one embodiment, in the particle system, the manipulated electrically charged particles are manipulated electrically charged pollen, and the system is configured to increase attraction forces toward the at least one plant that act on the manipulated electrically charged pollen.

According to one embodiment, in the particle system, the manipulated electrically charged particles are manipulated electrically charged pollen, and the system is configured to decrease attraction forces toward the at least one plant that act on the manipulated electrically charged pollen.

According to one embodiment, in the stationary plant system, the at least one power source is configured to provide an extra low voltage.

According to another aspect of the present subject matter, there is provided a stationary plant system for manipulating an electrical potential of the plurality of plants, the system comprising: at least one power source electrically connected to a plurality of plant electrodes and at least one grounding electrode with electrically conductive cables, wherein each plant electrode is configured to electrically and mechanically connect to a plant contact point at a plant, wherein each grounding electrode is configured to mechanically and electrically connect either to another plant contact point, or to a growth medium contact point in a growth medium in which the plurality of plants grow, and that the plurality of plants are in contact with the growth medium, or a combination thereof, wherein the connection of the plurality of plant electrodes and the at least one grounding electrode facilitates electrical current flow through the plurality of plants and wherein the electrical potential of the plurality of plants, or at least one part of a plant, is affected by inducing an electrical current in the electrical circuit.

According to one embodiment, in each plant of the plurality of plants, each at least one plant electrode is configured to electrically and mechanically connect to a plant contact point.

According to one embodiment, the plants are more than 1 meter apart. According to one embodiment, a number of the grounding electrodes is less than a number of the plant electrodes.

According to one embodiment, the plurality of plants is a plurality of trees, each comprising a trunk. According to one embodiment, a plant electrode is electrically and mechanically connected to a plant contact point at the trunk of the tree, substantially an edge of the trunk, above substantially a 50% trunk length.

According to one embodiment, a plurality of plant electrodes is configured to electrically and mechanically connect to corresponding plant contact points at the trunk of the tree, substantially at a same height of the trunk within substantially 10% trunk length, and around a circumference of the trunk.

According to one embodiment, a plurality of plant electrodes are configured to electrically and mechanically connect to one plant at different heights of the plant.

According to one embodiment, a distance between the power source and at least one plant electrode electrically and mechanically connected to at least one plant is larger than a distance between the power source and a closest plant to the power source.

According to one embodiment, a distance between the power source and at least one plant electrode electrically and mechanically connected to at least one plant is larger than 5 meters.

According to one embodiment, the plant electrode is configured to electrically and mechanically connect to inner tissue of the plant.

According to one embodiment, wherein the power source is configured to provide direct current (DC). According to one embodiment, the power source is configured to provide DC carrying alternating current (AC).

According to one embodiment, the stationary plant system further comprising a control unit configured to control an operation of the stationary plant system, to indicate or measure various parameters, and to communicate with components of the stationary plant system.

According to one embodiment, the control unit is configured to monitor at least one of ambient temperature; ambient humidity; wind conditions; density of pollen in air; direction and velocity of a pollen cloud in air; voltage, current, resistance in the combination system, and any combination thereof.

According to one embodiment, a portion of the electrically conductive cables are electrically insulated. According to one embodiment, the grounding electrode is an existing electrical ground.

According to one embodiment, the stationary plant system further comprising an in-growth medium electrically conductive cable that is configured to electrically connect to the power source and conduct an electrical current. According to one embodiment, the at least one power source is configured to provide an extra low voltage.

### BRIEF DESCRIPTION OF THE DRAWINGS

Embodiments are herein described, by way of example only, with reference to the accompanying drawings. With specific reference now to the drawings in detail, it is stressed that the particulars shown are by way of example and for purposes of illustrative discussion of the preferred embodiments, and are presented in the cause of providing what is believed to be the most useful and readily understood description of the principles and conceptual aspects of the embodiments. In this regard, no attempt is made to show structural details in more detail than is necessary for a fundamental understanding, the description taken with the drawings making apparent to those skilled in the art how several forms may be embodied in practice.

In the drawings:

FIGS. 1A-1B, schematically illustrate a combination system according to some embodiments of the invention;

FIG. 2A schematically illustrates a diagrammatic presentation of a prior art particle system according to some embodiments of the invention;

FIG. 2B schematically illustrates some additional embodiments of a prior art particle system;

FIG. 3 schematically illustrates a diagrammatic presentation of a prior art particle system, in a form of a system for dry artificial pollination of cultivated trees or shrubs by insect-borne pollen, according to some embodiments of the invention;

FIGS. 4A, 4B, 4C, 4D and 4E schematically illustrate a side view of a stationary plant system comprising a power source mechanically and electrically connected to at least one plant and to a growth medium, according to some embodiments of the invention;

FIG. 5A schematically illustrates a plant stationary system and its connection to a plurality of plants, according to some embodiments of the invention;

FIG. 5B is a flowchart of a method for manipulating an electrical potential of a plurality of plants according to some embodiments of the invention;

FIGS. 6A, 6B and 6C schematically illustrate a side view of some embodiments of a stationary plant system for manipulating an electrical potential of a plurality of plants, comprising a power source electrically connected to a plurality of plants in parallel, according to some embodiments of the invention;

FIGS. 7A and 7B schematically illustrate a side view of some embodiments of a stationary plant system, comprising a power source electrically connected to a plurality of plants in a series, according to some embodiments of the invention;

FIGS. 8A, 8B and 8C schematically illustrate plant electrodes according to some embodiments of the invention;

FIGS. 8D and 8E schematically illustrate plant electrodes assemble on a plat according to some embodiments of the invention;

FIG. 9A schematically illustrates a side view of a plant electrode configured to electrically and mechanically connect to at least one inner tissue, or layer of a plant, according to some embodiments of the invention;

FIGS. 9B, 9C and 9D, schematically illustrates a side view of plant electrodes configured to electrically and mechanically connect to at least one inner tissue, or layer of a plant, and electrically and mechanically connected to a plant, according to some embodiments of the invention;

FIGS. 10A, 10B and 10C schematically illustrate top longitudinal section views of various embodiments of a plant electrode according to some embodiments of the invention;

FIG. 11A schematically illustrates a side view of a plant electrode 20 comprising a plurality of affixing elements connected to a linear connector according to some embodiments of the invention;

FIG. 11B schematically illustrates a side view of a plant electrode comprising a plurality of affixing elements connected to a linear connector, electrically and mechanically connected to a plant according to some embodiments of the invention;

FIGS. 12A and 12B schematically illustrate a top view a plant electrode comprising at least one affixing element connected to a surface attaching connector according to some embodiments of the invention;

FIG. 13 schematically illustrates some definitions relating to a position of a plant contact point along a trunk of a tree according to some embodiments of the invention;

FIG. 14 schematically illustrates a position of plant electrodes on a trunk of a tree according to some embodiments of the invention;

FIG. 15 schematically illustrates t, a stationary plant system comprising a plurality of power sources according to some embodiments of the invention;

FIG. 16 schematically illustrates a depth of a grounding electrode in a growth medium according to some embodiments of the invention;

FIG. 17A schematically illustrates a side view of a mobile plant module, comprising a power source electrically connected to a plant and to a growth medium according to some embodiments of the invention;

FIG. 17B schematically illustrates mobile plant attaching elements attach to an arm through an arm connector according to some embodiments of the invention;

FIG. 18 schematically illustrates a side view of a plant electrification station, according to some embodiments of the invention;

FIG. 19 schematically illustrates a side view of two mobile plant module for manipulating an electrical potential of at least one plant, each mobile plant module comprising a flying mobile carrier according to some embodiments of the invention;

FIG. 20 schematically illustrates a side view a stationary plant module for manipulating an electrical potential of at least one plant, comprising a power source electrically connected to a plant at two contact points, according to some embodiments of the invention;

FIGS. 21A-21B schematically illustrate side views of a system for manipulating an electrical potential of at least one plant and for manipulating an electrical charge of particles that interact with the at least one plant, according to some embodiments of the invention;

FIG. 21C schematically a top view of a system for manipulating an electrical potential of at least one plant and for manipulating an electrical charge of particles that interact with the at least one plant, according to some embodiments of the invention; and

FIGS. 22A and 22B include graphs showing results of providing electricity to trees according to some embodiments of the invention.

### DESCRIPTION OF THE PREFERRED EMBODIMENTS

Before explaining at least one embodiment in detail, it is to be understood that the subject matter is not limited in its application to the details of construction and the arrangement of the components set forth in the following description or illustrated in the drawings. The subject matter is capable of other embodiments or of being practiced or carried out in various ways. Also, it is to be understood that the phraseology and terminology employed herein is for the purpose of description and should not be regarded as limiting. In discussion of the various figures described herein below, like numbers refer to like parts. The drawings are generally not to scale. For clarity, non-essential elements were omitted from some of the drawings.

It is an aspect of the present subject matter to provide a substitute or even replacement solution to natural pollination. In recent years, honey bees that facilitate the natural pollination are declining or disappearing all together, and therefore, the food industry that relies on pollination is endangered. The present subject matter provides a green solution to this problem by using technologies of artificial pollination (i.e., mechanical pollination) without harming nature.

Multiple plants have a natural electrical potential between the plant and the ground (earth), for example with a growth medium in which the plant is planted, or a growth medium that is in contact with the plant, e.g., soil in which the plant is planted; electrically charged liquid in which the plant is planted, like hydroponic plants; droplets of electrically charged liquid that are dispersed in the air in a vicinity of the plant (for example, water mist) and the like. The natural electrical potential changes overtime. These changes can be attributed to numerous reasons, such as environmental conditions, seasons, time of day, age of the plant, type of the plant, and the like.

Multiple plants also interact with particles. The term “particle”, as used herein, refers to any type of particle that is of interest in regard to the plant, that can be attracted to the plant, or to a part of the plant; or can be repelled from the plant, or from a part of the plant. Some particles that are desired to be more efficiently, and more effectively, attracted to plants include: a pollen grain, a droplet of liquid fertilizer, for example sprayed droplets of materials, and the like. Some particles that are desired to be more efficiently, and more effectively, repelled from the plant include: dust, a pest insect, a herbicide liquid droplet or powder particle that is to be repelled from an agricultural crop, and the like. Particle major diameter can vary significantly, from substantially 0.0001 micrometers to substantially 10,000 micrometers. Specifically, less than substantially 0.001, 0.01, 0.1, 0.5, 1.0 micrometers. Specifically, less than substantially 2, 5, 10, 15, 20, 30, 40, 50, 60, 70, 80, 90, 100, 150, 250, 500, 1,000, 10,000, 100,000, 500,000 micrometers.

According to one embodiment, the particle is an insect. According to another embodiment, the insect is a beneficial insect to the plant, for example a honey bee, a bumble bee, a butterfly, a moth, a wasp, a fly, and the like, that carries insect borne pollen grains. According to this embodiment, the aim of the present subject matter is to improve attraction of the beneficial insect to the plant, or to at least one part of the plant, for example at least one flower, or at least one stigma, and the like. According to an additional embodiment, the insect is a pollinating insect, and changing the electrical potential of the plant also improves the attraction of pollinating insects towards stigmas of flowers of the plant. According to a further embodiment, the insect is a pest insect. According to this embodiment, the present subject matter is aimed at repulsion the pest insect from the plant, or from at least one part of the plant, that is vulnerable to damage by pest insects, for example leaves, trunk, roots, and the like. As used herein, trunk also referred to as the stem, is the part of a tree, that connects the leafy crown with its roots. The crown of the tree is the upper part of the tree composed of leaves, twigs, branches, flowers and fruit. According to one embodiment, the insect is in the vicinity of the plant, for example flying in the air in the vicinity of the plant. According to yet an additional embodiment, the insect is on the plant, for example on a surface of at least one leaf, on a bark of a tree trunk, and the like. According to still an additional embodiment, the insect is in the plant, for example in inner tissues, or layers of a trunk of a tree, within a plant's epidermis tissue, in a plant's roots, and the like.

According to one embodiment, the present subject matter is aimed at attracting pollen grains that are carried by pollen carriers, for example insects, birds and other animals, to a plant. Pollen grains adhere to a body of the pollen carrier when the pollen carrier “visits” a flower of a plant, or passes by a flower of the plant. Then the pollen carrier moves to another flower, either of the same plant or of another plant, and the carried pollen grains that are carried by the pollen carrier are captured by a stigma of the other flower. The present subject matter is aimed at assisting with the attraction of the pollen grains by the stigmas and facilitate, and improve, attraction of the pollen grains that are carried by the pollen carriers by the stigmas.

Electrically charged particles are either attracted to the plant, or to a part of the plant; or repelled from the plant, or from a part of the plant, as a result of a difference in polarity and quantity between the electrical charge of the electrically charged particle and the electrical charge of the plant, or of the part of the plant. Control of the attraction, or repulsion, of the particles, to or from the plant, respectively, can be achieved by either manipulating an electrical potential of the plant, or of a part of the plant; or manipulating an electrical charge of the particles; or a combination thereof.

An aim of the present subject matter is to control either attraction to the plant, or to a part of the plant, or repulsion from the plant, or from a part of the plant, of electrically charged particles, by manipulating an electrical potential of the plant, alternatively in combination with manipulating an electrical charge of the particles.

Plants and specifically trees are considered to be very poor conductors of electrical current. In some cases, the plant can conduct electrical current, but its electrical resistance properties dominant. Different elements and layers of the plant exhibit substantially different conductivity capabilities. For example, the electrical resistance properties of the outer layer of most trees is very high. And often significantly higher than the internal layers of the trunk.

For achieving the aims of the present subject matter, there is provided a combination system for manipulating an electrical potential of at least one plant and manipulating an electrical charge of particles that interact with the at least one plant, the combination system comprising: at least one plant system for manipulating an electrical potential of at least one plant; and at least one particle system for manipulating an electrical charge of particles that interact with at least one plant.

The present subject matter further provides a combination system for manipulating an electrical potential of a plurality of plants and manipulating an electrical charge of particles that interact with at least one plant whose electrical charge has been manipulated, the combination system comprising: at least one stationary plant system for manipulating an electrical potential of a plurality of plants; and at least one particle system for manipulating an electrical charge of particles that interact with at least one plant whose electrical potential has been manipulated.

In addition, there is provided a method for manipulating an electrical potential of at least one plant and manipulating an electrical charge of particles that interact with the at least one plant, the method comprising: manipulating an electrical potential of at least one plant using at least one plant system; and manipulating an electrical charge of particles that interact with the at least one plant using at least one particle system. In other words, the present subject matter provides a method for using the combination system.

According to one embodiment, the plant system 1 is part of the combination system 12, and the method for using the plant system 1 is part of the method for using the combination system. According to another embodiment, the plant system 1 and the method for using the plant system 1 are independent and stand alone.

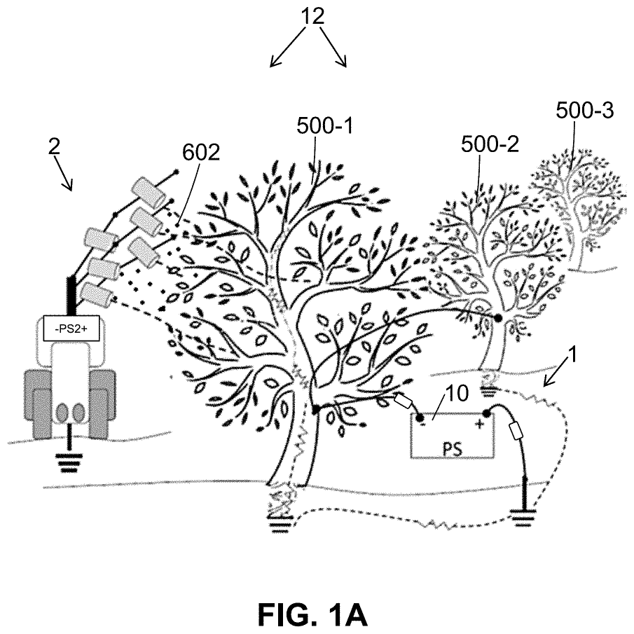

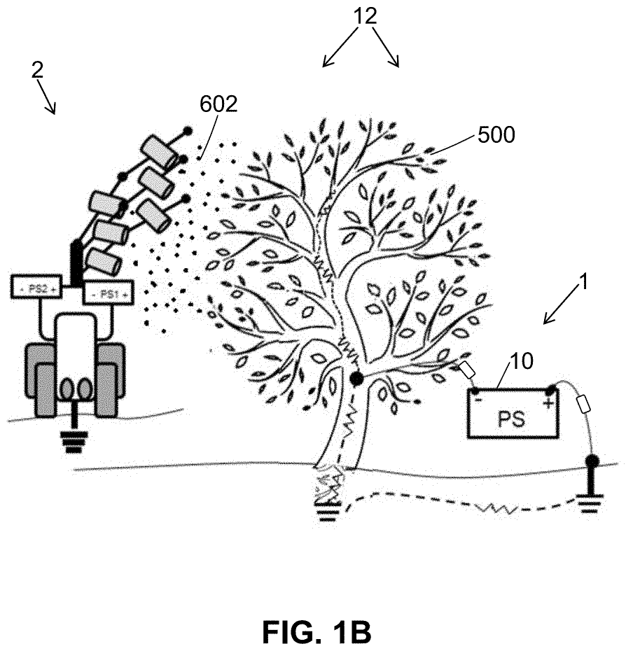

Referring now to FIGS. 1A-B, schematically illustrating a combination system according to some embodiments of the invention. According to one embodiment, the combination system 12 comprises a plant system 1 and a particle system 2. The plant system 1 is for manipulating an electrical potential of at least one plant 500, by providing electricity to the plant form power source 10 (e.g., a DC power source). In other words, the plant system 1 is configured to manipulate an electrical potential of at least one plant 500. Thus, FIG. 1A shows a plant system 1 that manipulates an electrical potential of a plurality of plant 500, and FIG. 1B shows a plant system 1 that manipulates an electrical potential of one plant 500. The particle system 2 is for manipulating an electrical charge of particles that interact with the at least one plant 500. In other words, the particle system 2 is configured to manipulate an electrical charge of particles that interact with the at least one plant 500 (i.e., a plurality of plant 500 as shown in FIG. 1A, or one plant 500 as shown in FIG. 1B). As a result of the manipulation of the electrical charge of the particles by the particle system 2, the particles become manipulated electrically charged particles 602, and they are dispersed by the particle system 2 towards the at least one plant 500.

Referring now to the particle system 2. An aim of the particle system 2 is to manipulate an electrical charge of particles 600 that interact with the at least one plant 500. This aim is achieved by either changing a quantity of an electrical charge of the particles 600, or changing a polarity of an electrical charge of the particles 600, or a combination thereof. Manipulating the electrical charge of the particles 600 affects movement of the particles 600 toward the at least one plant 500 with which the particles 600 are to interact. There are two types of movement of the particles 600 in relation to the at least one plant 500: attraction of the particles 600 to the at least one plant 500, and repulsion of the particles 600 from the at least one plant 500. Manipulating the electrical charge of the particles 600 affects, namely increases or decreases, the attraction of the particles 600 to the at least one plant 500, or the repulsion of the particles 600 from the at least one plant. In addition, manipulating the electrical charge of the particles 600 affect the trajectory and velocity of the movement of the particles 600.

Particles that it is desired to increase their attraction to the at least one plant assist in modifying and improving growth and health of the at least one plant and/or yield of products of the at least one plant. In some embodiments, beneficial particles to the at least one plant include, but not limited to, pollen grains, fertilizer particles, fluid droplets and the like.

Particles that it is desired to increase their repulsion from the at least one plant harm growth and health of the at least one plant and/or yield of products of the at least one plant, or cause damage to the at least one plant. In some embodiments, harmful, or damaging, particles to the at least one plant, include, but not limited to, pesticide particles, herbicide particles that their repulsion from a crop plant is desired when treating weeds that interfere with the growth and health of the crop plant, and the like.

The following terms are used hereinafter to distinguish between two types of particles: The term “natural electrically charged particles” as disclosed hereinafter refers to particles that have natural electrical charge characteristics, without any artificial intervention. The term “manipulated electrically charged particles” as disclosed hereinafter refers to particles whose electrical charge has been manipulated with the particle system 2 of the present subject matter, according to embodiments described herein.

Still referring to FIGS. 1A-B. According to one embodiment, the particle system 2 is configured to manipulate a natural electrical charge of the particles 600, to obtain manipulated electrically charged particles 600, and disperse the manipulated electrically charged particles 600. The term “to disperse”, or “dispersing” as disclosed herein refers to spreading, or distributing particles 600. Any mechanism of dispersing the particles 600 is under the scope of the present subject matter, for example, spraying, scattering, sprinkling, and the like, of the particles 600.

According to another embodiment, the manipulated electrically charged particles 600 are dispersed in a vicinity of the at least one plant 500 with which the manipulated electrically charged particles 600 interact. For example, pollen grains that are dispersed in the air in the vicinity of the at least one plant 500 as part of an artificial pollination process of the at least one plant 500, as shown in FIGS. 1A-B; fertilizer particles 600 that are dispersed in the air in the vicinity of the at least one plant 500; pesticide particles 600 that are dispersed in the air in the vicinity of the at least one plant 600, and the like.

Even though the particle system 2 of the present subject matter is known in the art, a brief description of the particle system 2, including a particle system 2 designed by the inventors of the present subject matter, is given below for the sake of better understanding the present subject matter.

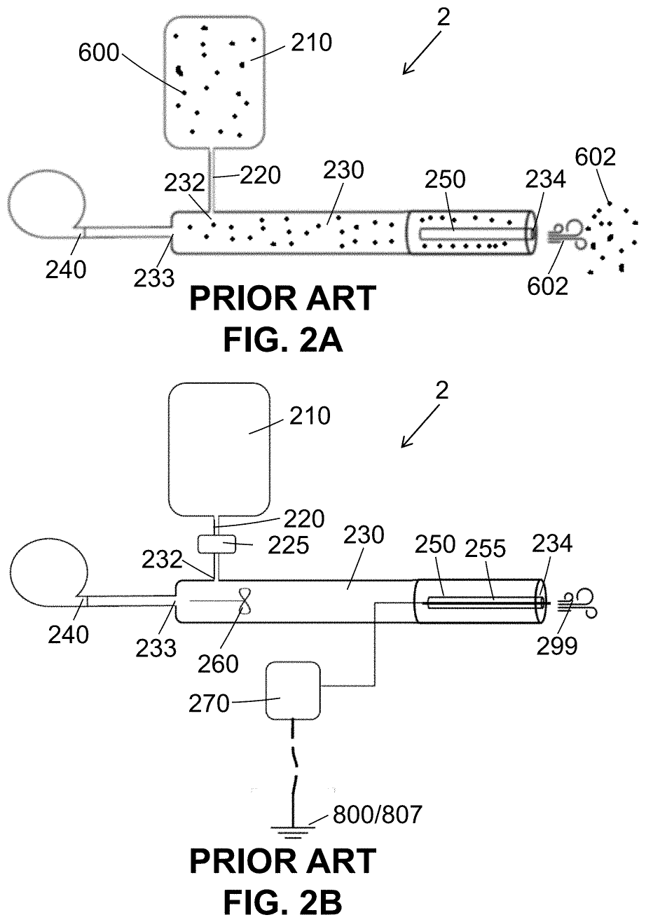

Referring now to FIG. 2A schematically illustrating a diagrammatic presentation of a prior art particle system. According to one embodiment, the particle system 2 comprises: at least one container 210 configured to accommodate particles 600; at least one particle distributor 230 configured to receive particles 600 from the container 210 and distribute the particles 600, wherein the at least one particle distributor 230 comprises: at least one particle inlet 232 in each of the at least one particle distributor 230, fluidically connected to the at least one container 210 with at least one conduit 220: at least one flow inlet 233 in each of the at least one particle distributor 230, positioned aside the at least one particle inlet 232; and at least one outlet 234 in each of the at least one particle distributor 230, positioned in an opposite side of the particle distributor 230 relative to the particle inlet 232 and the flow inlet 233, and configured to let particles 600 distribute out of the particle distributor 230; and at least one flow generator 240 fluidically connected to the at least one flow inlet 233 and configured to facilitate flow of the particles 600 from the at least one particle inlet 232, through the at least one particle distributor 230, toward the at least one outlet 234, wherein an electrical charge of the particles 600 is manipulated during flow of the particles 600 from the at least one container 210 to the at least one outlet 234, thereby forming manipulated electrically charged particles 602 that are distributed out through the at least one outlet 234.

According to one embodiment, the particles 600 flow from the container 210 to the conduit 220, then to the particle inlet 232, then through the particle distributor 230 to the outlet 234 and out of the particle system 2. According to another embodiment, the manipulated electrically charged particles 602 flow out of the particle system 2 in a vicinity of at least one plant. During the flow of the particles 600 through the particle system 2, the electrical charge of the particles 600 is manipulated, thus converting the particles 600 to manipulated electrically charged particles 602, as can be seen in FIG. 2A.

According to one embodiment, the container 210 is configured to accommodate particles 600. In some embodiments, particles 600 include: solid particles 600 of any size, for example pollen aggregates, granules of a solid fertilizer, or pesticide, or herbicide, and the like; a powder, for example a powder of pollen, and the like. According to another embodiment, the container 210 is configured to accommodate a liquid that is to be dispersed as particles 600, for example particles 600 in a form of droplets. Some examples of a liquid include, but not limited to, a liquid solution of a fertilizer, or a pesticide, or a herbicide and the like.

The term “pollen” as disclosed herein refers to a powdery substance produced by seed plants. Pollen comprises pollen grains, which produce male gametes, also known as sperm cells.

The term “pesticide” as disclosed herein refers to a substance for controlling pests that can cause damage to plants. In other words, a pesticide, as referred to herein, is a substance that serves as a plant protection product, or a crop protection product, which in general, protects plants from weeds, fungi, or insects. The pesticide can be a chemical substance, or a biological agent, for example a virus, bacterium, or fungus, that deters, incapacitates, kills, or otherwise discourages pests. Another type of pesticides is based on mineral oils for the treatment of trees infected with acari, for example; or dormant oils that are applied on plants during dormancy, for example at winter, in order to eradicate pests. In some embodiments, target pests of a pesticide can include insects, plant pathogens, weeds, mollusks, birds, mammals, fish, nematodes (roundworms), and microbes that destroy property, cause nuisance, or spread disease, or are disease vectors.

The term “fertilizer” as disclosed herein refers to any material of natural or synthetic origin that is applied to soil or to plant tissues to supply plant nutrients. The most common fertilizers include three main macro nutrients: Nitrogen (N), Phosphorus (P), and Potassium (K) with occasional addition of supplements like rock dust for micronutrients. The fertilizer is applied to the plants in a variety of forms, just to mention a few forms relevant for the present subject matter: as a dry powder, or dissolved in liquid and applied as aerosols or mist.

According to one embodiment, the conduit 220 is configured to guide flow of the particles 600 from the container 210 to the particle inlet 232 of the particle distributor 230. According to another embodiment, the conduit 220 is configured to determine a dosage of the particles 600 that flow from the container 210 to the particle inlet 232 of the particle distributor 230. For example, a diameter of the conduit 220 can determine the dosage of the particles 600. The higher the diameter of the conduit 220—the higher is the dosage of the particles 600. Other types of dosing elements that are configured to determine the dosage of the particles 600 are described in FIG. 2B.

According to one embodiment, the flow generator 240 is configured to facilitate flow of the particles 600 from the particle inlet 232 toward the outlet 234 through the particle distributor 230. According to another embodiment, the flow generator 240 is configured to increase the flow of the particles 600 through the particle distributor 230. Any type of flow generator 240 that is configured to perform the aforementioned functions is under the scope of the present subject matter. In some embodiments, flow generators 240 include, but not limited to: a blower; a vent; a high pressure flow generator; a pump; a device that allows falling of the particles 600 by gravitation thereby facilitating, and even increasing, the flow of the particles 600; a rotating device that by its rotation facilitates, and even increases, the flow of the particles 600, and the like.

As mentioned above, according to one embodiment, the electrical charge of the particles 600 is manipulated during flow of the particles 600 from the container 210 to the outlet 234, thereby forming manipulated electrically charged particles 602. Any type of manipulation of the electrical charge of the particles 600 is under the scope of the present subject matter. For example, usage of a phenomenon known as the triboelectric effect, or triboelectric charging. This can be achieved, for example, by the friction of the particles 600 with components of the particle system 2 through which the particles 600 flow. For example, friction of the particles 600 with walls of the container 210, or friction of the particles 600 with the conduit 220, or the particle distributor 230, or other components though which the particles 600 flow. For example, an inner aspect of the particle distributor 230 comprises negatively charging materials like glass, aluminum, nylon, mica and the like. Particles 600 that flow through the particle distributor 230 and rub, or come in contact with the negatively charging materials, become negatively charged. For another example, an inner aspect of the particle distributor 230 comprises positively charging materials like polytetrafluoroethylene (PTFE), Teflon, silicon, polyvinyl chloride (PVC) and the like. Particles 600 that flow through the particle distributor 230 and rub, or come in contact with, the positively charging materials become positively charged.

According to another embodiment, the particle system 2 further comprises a charger 250 configured to manipulate the electrical charge of the particles 600. Any type of manipulation of the electrical charge of the particles 600 is under the scope of the present subject matter. In one example, manipulation of the electrical charge of the particles 600 is taking particles 600 that are neutral, namely have no electrical charge, and cause the particles 500 to become electrically charged, either positively, or negatively. In another example, manipulation of the electrical charge of the particles 600 is changing a polarity of the electrical charge of the particles 600. If the particles 600 are positively charged, the manipulation of their electrical charge converts the particles 600 to negatively charged, and vice versa. In yet another example, manipulation of the electrical charge of the particles 600 is changing a quantity of the electrical charge of the particles 600, which according to the International System of Units (SI) is measured in coulombs. Thus, manipulation of the electrical charge of the particles 600 can be either increasing, or decreasing, the quantity of the electrical charge of the particles 600. In still another example, manipulation of the electrical charge of the particles 600 is any combination of the aforementioned types of manipulation of the electrical charge of the particles 600.

According to yet another embodiment, the charger 250 is positioned at any site of the particle system 2 where particles 600 flow, for example: in the container 210, or in the conduit 220, or in the particle distributor 230. According to still another embodiment, the charger 250 is positioned in the particle distributor 230, in a vicinity of the outlet 234, as shown in FIG. 2A. According to a further embodiment, a distal end of the charger 250 protrudes from the outlet 234 of the particle distributor 230. According to yet a further embodiment, the distal end of the charger 250 is in-line with the outlet 234 of the particle distributor 230. According to still a further embodiment, the distal end of the charger 250 is within the particle distributor 230. Any type of charger 250 is under the scope of the present subject matter. For example, in relation to the usage of the triboelectric charging described above, the charger 250 can be a nozzle. According to this embodiment, the nozzle is made of a material that causes electrical charging of the particles 600 that rub with the nozzle, for example a nozzle made of glass for charging the particles 600 with a negative electrical charge, or a nozzle made of polytetrafluoroethylene (PTFE) for charging the particles 600 with a positive electrical charge.

Additional embodiments of manipulating the electrical charge of the particles 600 are described in FIG. 2B.

Referring now to FIG. 2B schematically illustrating some additional embodiments of a prior art particle system. For clarity, non-essential elements, previously described, have been omitted.

As mentioned above, the diameter of the conduit 220 can determine the dosage of the particles that flow from the container 210 to the particle inlet 232 of the particle distributor 230. According to another embodiment, the particle system 2 further comprises a dosing element 225 configured to determine the dosage of the particles that enter into the particle inlet 232 of the particle distributor 230. According to yet another embodiment, the dosing element 225 is positioned in fluid connection with the conduit 220 in a manner that allows the dosing element 225 to determine the dosage of the particles that flow either to, or through, or from the conduit 220. Thus, according to one embodiment, the dosing element 225 is positioned in the container 210 and is configured to determine the dosage of the particles that flow to the conduit 220. According to another embodiment, the dosing element 225 is positioned on the conduit 220 and is configured to determine the dosage of the particles that flow through the conduit 220. According to yet another embodiment, the dosing element 225 is positioned at the particle inlet 232, or on the conduit 220 adjacent to the particle inlet 232, and is configured to determine the dosage of the particle that flow from the conduit 220 to the particle distributor 230.

Any type of dosing element 225 is under the scope of the present subject matter. In some embodiments, dosing elements 225 include: a dosing element 225 that operates by motion of a disk; a nozzle through which the particles flow—with alternatively an element applying high pressure on the flow of the particles. According to one embodiment, the dosing element 225 is configured to generate microdoses of the particles that enter the inlet zone 232. According to another embodiment, the dosing element 225 is configured to allow passage of particles having a certain size, or size range, while preventing passage of particles that do not have a desired size, or size range. According to yet another embodiment, the dosing element 225 is configured to allow passage of particles having a certain shape, while preventing passage of particles not having a desired size.

It can be understood from the description thus far that the particles flow in the particle system 2 from the container 210 to the outlet 234 and out of the particle system 2, for example toward at least one plant. Accordingly, the particles are in a fluidic form, and flow in a particle environs. The particle environs can be a gas, or a mixture of gases, for example atmospheric air. Airborne pollen, for example, are particles that flow in a particle environs in a form of atmospheric air. In another example, the particles can be a liquid, for example a liquid solution of pesticides, or herbicides, or a fertilizer. In this embodiment, the particles are droplets of the liquid and the particle environs is also a gas, or a mixture of gases, for example atmospheric air. In this form, the particles are dispersed as aerosols, or mist. In all these embodiments, there is an optional need to mix the particles with the particle environs.

Thus, according to one embodiment, the particle system 2 further comprises a mixer 260, as illustrated in FIG. 2B. The mixer 260 is configured to mix the particles together with the particle environs, for example in order to ensure homogeneous dispersion of the particles and as much as possible uniform manipulation of the electrical charge of the particle. According to another embodiment, the mixer 260 is positioned inside the particle distributor 230. According to yet another embodiment, the mixer 260 is positioned in a vicinity to the particle inlet 232 and the flow inlet 233 of the particle distributor 230, for example in order to mix the particles with the particle environs upon entry of the particles into the particle distributor 230.

As mentioned above and shown in FIG. 2A, the particle system 2 comprises a charger 250 configured to manipulate the electrical charge of the particles. Several embodiments of the charger 250 were mentioned above as well. According to the aforementioned embodiments, the charger 250 operates per se. According to an additional embodiment shown in FIG. 2B, the charger 250 operates by using at least one particle power source 270 that functions as a source of electrical current, or electrical voltage, for electrically charging the particles. Thus, the charger 250 functions as an electrode, or the charger 250 further comprises at least one electrode 255, as shown in FIG. 2B. Thus, according to one embodiment, the charger 250 is an electrode, or the particle system 2 further comprises at least one electrode 255 at the charger 250, and at least one particle power source 270 electrically connected to the charger 250 functioning as an electrode, or to the at least one electrode 255. In addition, the at least one particle power source 270 is electrically connected to a particle electrical ground 807, for example soil 800, wherein the charger 250 functioning as an electrode, or the at least one electrode 255, is configured to manipulate the electrical charge of the particles, and the particle power source 270 is configured to supply an electrical power, for example in a form of an electrical current, or an electrical voltage, to the charger 250 functioning as an electrode, or to the at least one electrode 255. According to another embodiment, the at least one electrode 255 is configured to electrically charge the particles.

According to one embodiment, the particle power source 270 is configured to provide various levels of different characteristics of electrical power, for example various levels of electrical current, various levels of electrical voltage, and the like. Any type of mechanism, and any composition of the particle power source 270, that allows the particle power source 270 to provide the various levels of different characteristics of electrical power, is under the scope of the present subject matter. Following are some embodiments of the particle power source 10 that allow the particle power source 10 to provide the various levels of different characteristics of electrical power: According to one embodiment, the particle power source 270 comprises at least two resistors. According to another embodiment, the particle power source 270 comprises multiple capacitors. According to yet another embodiment, the particle power source 270 comprises at least two resistors and multiple capacitors.

Any type of electrode 255 that is configured to manipulate an electrical charge of flowing particle, and more particularly electrically charge the flowing particles, is under the scope of the present subject matter, for example a net electrode 255. The net electrode 255 has a shape of a net that is electrically charged. Passage of the flowing particles through the net electrode 255 manipulates the electrical charge of the particles, for example electrically charges the particles, or changes a quantity of an electrical charge of the particles, or changes a polarity of the electrical charge of the particles, or any combination thereof. Thus, according to one embodiment, the electrode 255 is a net electrode 255.

Another example is a corona-discharge electrode 255. Thus, according to another embodiment, the electrode 255 is a corona-discharge electrode 255.

A corona-discharge electrode 255 is a conductor configured to carry high electrical voltage. When a high electrical voltage is carried by the corona-discharge electrode 255, a fluid, for example a particle environs, that surrounds the corona-discharge electrode 255, for example air, is ionized, and as a result the air undergoes electrical breakdown and become electrically conductive. When particles are carried by the air, they are electrically charged as well. Therefore, when the charger 250 comprises a corona-discharge electrode 255, a mixture of ionized air and manipulated electrically charged particles 602 is distributed through the outlet 234 of the particle distributor 230.

According to one embodiment, the particle power source 270 is configured to supply a high voltage to the at least one electrode 255, for example when the electrode 255 is a corona-discharge electrode 255.

According to one embodiment, the at least one electrode 255 is positioned on a longitudinal axis of the particle distributor 230. According to another embodiment, the at least one electrode 255 is a single electrode 255. According to yet another embodiment, the at least one electrode is a multiplicity of electrodes 255. According to still another embodiment, the multiplicity of electrodes 255 are arranged substantially parallel to each other. According to a further embodiment, the multiplicity of electrodes 255 are electrically connected to each other. According to yet a further embodiment, the at least one electrode 255 is located on a circumference of the outlet 234. According to still a further embodiment, the at least one electrode 255 protrudes out of the outlet 234.

According to one embodiment, a temperature of the particle environs is kept above substantially 5, 10, 25, 20, 25, 30, 45, 50, 60, 70 degrees Celsius. According to another embodiment, the temperature of the particle environs is kept at ambient temperature+/− (plus/minus) substantially 1, 2, 3, 4, 5, 7, 10, 25 degrees Celsius, as measured in a period of less than substantially 0.1, 0.2, 0.5, 1, 2, 3, 4, 5, 6 hours. According to yet another embodiment, the particles temperature is kept above substantially 5, 10, 25, 20, 25, 30, 45, 50, 60 degrees Celsius. According to still embodiment, the particles temperature is kept at an ambient temperature+/−(plus/minus) substantially 1, 2, 3, 4, 5, 7, 10, 25 degrees Celsius, as measured in a period of less than substantially 0.1, 0.2, 0.5, 1, 2, 3, 4, 5, 6 hours.

According to one embodiment, a humidity of the particle environs is kept above substantially 2, 5, 10, 25, 20, 25, 30, 45, 50, 60, 70, 80% (v/v). According to another embodiment, the humidity of the particle environs is kept at an ambient humidity level+/− (plus/minus) substantially 1, 2, 3, 4, 5, 7, 10, 25, 35, 50. 60 70% (v/v), as measured in a period of less than substantially 0.1, 0.2, 0.5, 1, 2, 3, 4, 5, 6 hours. According to yet another embodiment, the humidity of the particles in the container 210 is kept above substantially 2, 5, 10, 25, 20, 25, 30, 45, 50, 60, 70% (v/v). According to still another embodiment, the humidity of the particles in the container 210 is kept above an ambient humidity level+/−(plus/minus) 2, 5, 10, 25, 20, 25, 30, 45, 50% (v/v) as measured in a period of less than substantially 0.1, 0.2, 0.5, 1, 2, 3, 4, 5, 6 hours.

According to one embodiment, components of the particle system 2 are heat insulated. This embodiment can be achieved, for example, by an embodiment according to which the components of the particle system 2 are made, at least partially, of temperature insulating materials. In some embodiments, components of the particle system 2 that can be heat insulated include: the container 210, the conduit 220, the particle distributor 230 and the like.

According to one embodiment, the particle system 2 comprises at least one humidity control element configured to control the humidity of the particles that are in the particle system 2. Any type of humidity control element is under the scope of the present subject matter. According to one embodiment, the humidity control element is an active humidity control element. According to another embodiment, the humidity control element is a passive humidity control element. According to yet another embodiment, the particle system 2 comprises multiple humidity control elements, wherein at least one humidity control element is an active humidity control element and at least one humidity control element is a passive humidity control element.

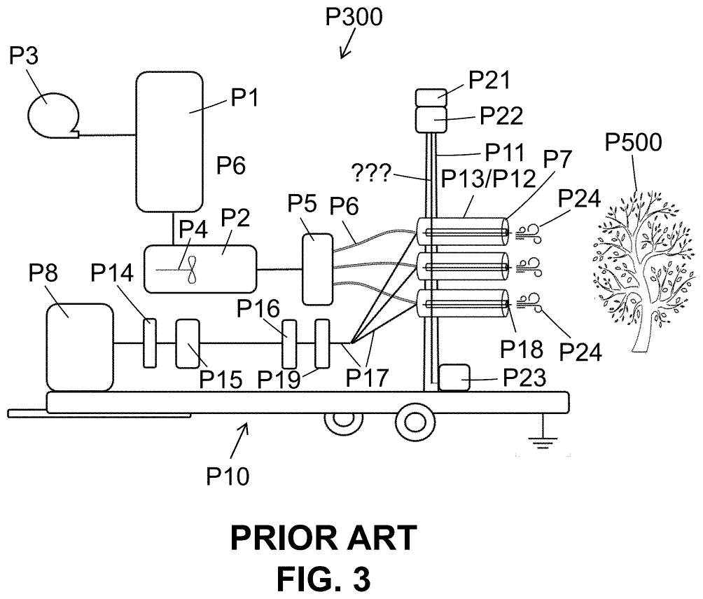

Referring now to FIG. 3, schematically illustrating a diagrammatic presentation of a prior art particle system, in a form of a system for dry artificial pollination of cultivated trees or shrubs by insect-borne pollen, according to some embodiments of the invention. It should be noted that the term “artificial pollination” is synonymous with “mechanical pollination”. This system, and a method for using said system, are described in United States patent application No. U.S. Ser. No. 16/644,900 (publication No. US 2020/0260675), the entire contents of which is incorporated herein by reference in its entirety. It should be noted that the terms used in the description of FIG. 3 are similar to the terms described in US 2020/0260675, while their reference numbers are substantially in accordance with the reference numbers used in US 2020/0260675. However, as one may understand, the system illustrated in FIG. 3 is a specific embodiment of the particle system 2 illustrated in FIGS. 2A-B.

FIG. 3 illustrates a particle charging and distribution system P300, configured to electrically charge and distribute pollen. In the system P300 illustrated in FIG. 3, an air supply P3 feeds compressed air to feeding system P2 through particle container P1. Then, the particles move to feeding system P2. Feeding system P2 comprises a mixer P4. The particles are moved by compressed air, or by venturi effect, via the mixer P4 such that particles are mixed with the compressed air in a homogenous manner. Then, an air-particles mixture is fed to distributer P5 that is configured to distribute the air-particles mixture over nozzles P7 via pipes P6. Reference numbers P13 and P18 refer to external shields and central electrodes, respectively. Manipulated electrically charged particles cloud P24 is directed to a target area P500. Electrical charging of the particles can be performed by at least one alternative, such as charging in the container P13, by corona discharge by electrode P18, or by a triboelectric effect based on friction. System P300 is mounted on chassis P10 that can be self-propelled, or manually movable. In the case of the self-propelled embodiment, the system P300 is provided with a propulsion system (not shown). Reference number P8 refers to a power supply. Electric circuitry is energized via circuit breaker P14, converter P15, high voltage distribution unit P16, high voltage safety unit P19, and conduction system P17. A plurality of electrostatic barrels P12 is organized in an array.

Barrel mounting pole P11 is configured to mount at least one electrostatic barrel. According to one embodiment, barrel mounting pole P11 is configured to mount at least one sensing unit of meteorological variables P21. According to one embodiment, the barrel mounting pole P11 is configured to mount at least one sensing unit of spatial parameters P22.

According to one embodiment, the at least one sensing unit of meteorological variables P21 is configured to sense meteorological variables such as wind velocity and direction, air temperature, relative humidity and luminance. According to another embodiment, at least one sensing unit of spatial parameters P22 is configured to identify target areas P500, such as plants, and relative position of a target to a particle charging and distribution system P300, and build a three-dimensional model of a target. Any type of sensing unit of spatial parameters P22 is under the scope of the present subject matter, for example, but not limited to, a Light Detection and Ranging (Lidar) system; any type of imaging device, for example a video imaging device, a still imaging device, and the like. Processing unit P23 is signally connected to a control unit, and is configured to control at least one of the following parameters: flow velocity of the particles within the at least one electrostatic barrel, voltage on an electrode within the electrostatic barrel, dispensable dose of the particles, distance between the electrostatic barrel and the target, direction of the flow of the particles, position of the system P300 relative to the target area P500, and the like.

Referring now to the plant system 1, a schematic illustration of which is given in FIG. 1. The present subject matter provides a plant system 1 for manipulating an electrical potential of at least one plant by forming an electrical circuit that includes the at least one plant.

In other words, the present subject matter provides a plant system 1 that is configured to manipulate an electrical potential of at least one plant by forming an electrical circuit that includes the at least one plant.

In addition, the present subject matter provides a method for manipulating an electrical potential of at least one plant by forming an electrical circuit that includes the at least one plant, by using the plant system 1. In other words, the present subject matter provides a method for using the plant system 1.

The term “electrical circuit” as disclosed herein refers to a closed loop path for transmitting electrical current from a power source through at least one plant and back to the power source. In some embodiments, the electrical circuit includes in addition a growth medium in which the at least one plant grows. The growth medium is also in mechanical and electrical contact with the at least one plant. It should be noted that even though the electrical circuit includes components of the plant system 1, at least one plant and optionally the aforementioned growth medium, the at least one plant and the growth medium are not part of the present subject matter. The present subject matter includes the components of the plant system 1 that are for manipulating the electrical potential of the at least one plant. These components will be described in detail hereinafter.

One aim of the plant system 1 is to control the level of electrical potential, namely increasing or decreasing the electrical potential between the at least one plant and a growth medium in which the at least one plant grows and is in contact with the at least one plant. Another aim of the plant system 1 is to control the electrical potential of the at least one plant, or of different parts of the at least one plant, specifically of edges of the at least one plant, for example flowers, stigmas of flowers, and the like. Yet another aim of the plant system 1 is to control the duration of formation of an electrical circuit that causes the manipulation of the electrical potential of the at least one plant, or parts thereof.

The plant system 1 is configured to manipulate an electrical potential of at least one plant, thereby affecting either attraction of particles to the at least one plant, or repulsion of particles from the at least one plant. More particularly, the manipulation of the electrical potential of the at least one plant with the plant system 1 improves either attraction of particles to the at least one plant, or repulsion of particles from the at least one plant. The electrical potential of the at least one plant that is formed due to the manipulation of the electrical potential of the at least one plant is occasionally termed hereinafter “manipulated plant electrical potential”.

According to one embodiment, manipulating the plant electrical potential is changing the electrical potential of the at least one plant, and more specifically changing the intensity, or level, of the plant electrical potential, or changing the polarity of the plant electrical potential, or both changing the intensity and polarity of the plant electrical potential. According to another embodiment, the term “manipulating” refers to controlling the plant electrical potential of the at least one plant, namely deliberately changing the plant electrical potential of the at least one plant to a desired intensity, or frequency, or polarity, or any combination of desired intensity, frequency and polarity, and optionally keeping them for a desired duration of time. According to yet another embodiment, the term “manipulating” refers to monitoring the plant electrical potential of the at least one plant, namely registering the plant electrical potential of the at least one plant at certain points in time, or during a certain period of time. According to a further embodiment, the term “manipulating” refers to using closed loop control techniques. According to yet a further embodiment, the term “manipulating” refers to using closed loop control techniques comprising feedback and feed-forward signals. According to still another embodiment, the term “manipulating” refers specifically to “manipulating a plant electrochemical potential of at least one plant”.

In some embodiments, manipulating the plant electrical potential may be conducted to increase the provision of nutritive and fertilizing materials to the plant's roots.

Any type of plant is under the scope of the present subject matter, for example: a tree, a bush, a shrub, a herb, a grass and the like. More particularly, the plant is beneficial, for example an agricultural plant, an ornamental plant, and the like According to one embodiment, the plant system 1 is configured to manipulate the electrical potential of a part of a plant, for example a root, a trunk of a tree, a stem of a herb, a branch, a twig, a leaf, a flower, a stigma, a stamen, and the like, or a plurality of parts of the plant. According to another embodiment, the plant system 1 is configured to manipulate a segment of a part of the plant, for example a tip of a leaf, a segment of a branch, or twig, that is close to a surface of a crown of a tree, and the like.

As mentioned above, the plant system is configured to manipulate the electrical potential of at least one plant, and thereby affect either attraction of particles to the at least one plant, or repulsion of particles from the at least one plant. In other words, the electrical potential of a plant that is manipulated by the plant system can either increase or decrease, either attraction of particles to the plant, or repulsion of particles from the plant. This can be achieved when the particles are electrically charged. According to one embodiment, the manipulated electrical potential of the plant allows either attraction, or repulsion, of any particle, to or from, the plant, respectively. According to another embodiment, the particle is at a distance from the plant that allows the manipulated electrical potential of the plant to affect either attraction, or repulsion, of the particle, to or from the plant, respectively. According to yet another embodiment, the particle is in close vicinity to the plant.

According to one embodiment, the part of the plant is a stigma of a flower. Thus, according to this embodiment, the plant system is configured to affect either attraction, or repulsion, of particles, to or from at least one stigma, respectively. According to another embodiment, the particles are pollen grains. Thus, according to this embodiment, the plant system is configured to affect either attraction, or repulsion, of pollen grains to, or from, at least one stigma, respectively.

Multiple types of pollen grains are under the scope of the present subject matter, including insect-borne pollen, wind-borne pollen (also known as airborne pollen), animal-borne pollen, and the like. According to a further embodiment, the pollen grains are airborne. This embodiment relates to plants that are pollinated by airborne pollen grains, for example date trees, olive trees, pistachio trees, and the like.

According to yet a further embodiment, the pollen grains are insect borne, and the manipulated electrical potential of the at least one stigma facilitates improved attraction of the pollen grains from the insect toward the at least one stigma, or improved repulsion of the pollen grains away from the at least one stigma. This embodiment relates to plants that are pollinated by insects, birds or other animals carrying the pollen grains, for example, citrus trees (e.g., orange, lemon, grapefruit), mango trees, almond trees, and the like. According to still a further embodiment, the pollen grains are inherently insect-/bird-/animal-borne, but the pollen grains can be artificially spread toward, or near, at least one stigma of a plant, as airborne particles. According to an additional embodiment, the pollen grains are inherently insect-/bird-/animal-borne, but the insect-/bird-/animal-borne pollen grains are harvested, electrically charged and artificially spread toward, or near, at least one stigma of at least one plant, as airborne particles. It should be noted that these embodiments relate not only to at least one stigma, but also to at least one entire flower.

Here is a noncomprehensive list of some plants, that according to some embodiments, the plant system 1 of the present subject matter is configured to facilitate their pollination: Acerola, Adzuki Bean, Alfalfa, Allspice, Almond, Alsike Clover, Apple, Apricot, Areca Nuts, Arrowleaf Clover, Avocado, Azarole, Bambara Pea, Beans, Beet, Bell Pepper, Berries Spp., Black Currant, Blackberry, Blackeye Bean, Black-Eyed Pea, Blueberry, Boysenberry, Brazil Nut, Broad Bean, Broad Beans, Broccoli, Brussels Sprouts, Buckwheat, Cabbage, Cactus, Cajan Pea, Canola, Cantaloupe, Carambola, Caraway, Cardamom, Carrot, Cashew, Cashew Apple, Cauliflower, Celery, Cherry Spp., Chestnut, Chili Pepper Spp., Chinese Cabbage, Citrus Fruits, Clementine, Clover, Cocoa Beans, Coconut, Coffea Spp., Congo Bean, Coriander, Corn, Cotton, Cow Bean, Cowpea, Cranberry, Crimson Clover, Christmas tree, Crownvetch, Cucumber, Dogroses, Dry Beans, Durian, Eggplant, Elderberry, Feijoa, Fennel, Figs, Flax, Gherkins, Goa Bean, Gooseberries, Gourd, Grape, Grapefruit, Green Bean, Green Pepper, Greengage, Groundnuts, Guar Bean, Guava, Haricot Bean, Hazelnut, Hog Plum, Horse Bean, Hyacinth Bean, Jack Bean, Jujube, Karate Nuts, Karite, Kidney Bean, Cannabis, Kola Nuts, Lemon, Lima Bean, Lime, Linseed, Longan, Loquat, Lupine, Lychee, Macadamia, Mammee Apple, Mandarins, Mango, Mangoes, Mangosteens, Maracuja (Passion Fruit), Marrow, Melon, Melon Seed, Mirabelle, Mungo Bean, Mustard, Naranjillo, Nectarine, Okra, Onion, Orange, Papaya, Peach, Pear, Pecan, Peppers, Persimmon, Pigeon Pea, Pistachio, Plum, Pomegranate, Pomelos, Potato, Prickly Pear, Pumpkin, Quince, Rambutan, Rapeseed, Raspberry, Red Clover, Red Currant, Red Pepper, Rose Hips, Rowanberry, Safflower, Sainfoin, Scarlet Runner Bean, Service Tree (Sorbus Domestica), Sesame, Shea Nuts, Sloe, Soybean Spp., Squash (Plant), Starfruit, Starfruit Turnip, Strawberry, Strawberry Tree, String Bean, String Beans, Sunflower, Sword Bean, Tamarind, Tangelo, Tangerine, Tomato, Turnip, Vanilla, Vetch, Walnut, Watermelon, Wheat, White Clover, Zucchini.

Some additional plants, that the plant system 1 of the present subject matter is configured to facilitate their pollination, include: Grasses, grass, weed, wheat, Poaceae, Gramineae, and Corn.

The following table lists properties of some of the plants:

### Crops Pollinator Commercial Latin Name

product of
pollination
Kiwifruit Honey bees, Bumblebees, Solitary bees fruit Actinidia deliciosa
Almond Honey bees, Bumblebees, Solitary bees nut Prunus dulcis, Prunus
(Osmia cornuta), Flies amygdalus, or
Amygdalus communis
Mustard Honey bees, Solitary bees (Osmia seed Brassica alba, Brassica
cornifrons, Osmia lignaria) hirta, Brassica nigra
Cotton Honey bees, Bumblebees, Solitary bees seed, fiber Gossypium spp. Pistacia vera
Pistachio Wind pollination nut Pistacia vera
Pear Honey bees, Bumblebees, Solitary fruit Pyrus communis
bees, Hover flies (Eristalis spp.)
Wheat Wind Pollination grains Triticum Spp. (species)
Avocado Stingless bees, Solitary bees, Honey fruit Persea americana
bees
Cannabis, Wind pollination, Flowers, Cannabis Spp.
(Marijuana) seeds

According to one embodiment, the plant system is configured to form an electrical circuit, wherein the plant is a component of the electrical circuit, serving as a resistor, an inductor and capacitor. It should be emphasized that even through the plant system forms an electrical circuit in which electrical current can flow through the plant, and the plant is part of the electrical circuit, the plant is not part of the plant system. In this configuration, the electrical potential of the plant can be manipulated, and accordingly, the plant impedance (Z) can be manipulated as well. According to another embodiment, the electrical potential of the plant can be manipulated by the plant system of the present subject matter, and accordingly, the electrical current in the electrical circuit, including the plant, can be manipulated as well.

According to one embodiment, the plant system 1 is a stationary plant system for manipulating an electrical potential of a plurality of plants. In other words, the stationary plant system is configured to manipulate an electrical potential of a plurality of plants. According to another embodiment, the plant system 1 is a mobile plant system for manipulating an electrical potential of at least one plant at a time. In other words, the mobile plant system is configured to manipulate an electrical potential of at least one plant at a time.

According to one embodiment, in the combination system 12, an operation of the stationary plant system and the mobile plant system is coordinated with an operation of the at least one particle system 2.

Referring now to the stationary plant system. The present subject matter provides a stationary plant system for manipulating an electrical potential of a plurality of plants by forming an electrical circuit in which an electrical current can flow through the plurality of plants, the stationary plant system comprising: at least one power source electrically connected to a plurality of plant electrodes (e.g., first electrodes) and at least one grounding electrode, wherein each plant electrode is configured to electrically and mechanically connect to a plant contact point in a plant, wherein each grounding electrode is configured to mechanically and electrically connect either to a plant contact point, or to a growth medium contact point in a growth medium in which the plurality of plants grow, and that the plurality of plants are in contact with the growth medium, or a combination thereof, wherein the connection of the plurality of plant electrodes and the at least one grounding electrode causes formation of an electrical circuit in which electrical current can flow through the plurality of plants and wherein the electrical potential of the plurality of plants, or at least one part of a plant, is affected by inducing an electrical current in the electrical circuit thus providing an electrical potential between the plants and the growth medium.

It should be noted that the plant contact point is not part of the stationary plant system, and is not under the scope of the present subject matter. A plant contact point is defined as a spot on a surface of a plant, or a spot in an interior of a plant, for example at least one inner tissue, or layer of the plant, to which a plant electrode, and occasionally a grounding electrode, is mechanically and electrically connected.

Mechanical connection of the plant electrode, or grounding electrode, to the plant contact point means that there is a physical connection between the plant electrode, or grounding electrode, and the plant contact point. In some embodiments, there is no gap between the plant electrode, or the grounding electrode, and the plant contact point. More specifically, there is no gap of air between the plant electrode, or grounding electrode, and the plant contact point. According to one embodiment, the physical connection between the plant electrode, or the grounding electrode, and the plant contact point is robust. According to another embodiments, there is a need to invest force in order to separate the plant electrode, or the grounding electrode, from the plant contact point. According to a further embodiment, the plant contact point is in at least one inner tissue, or layer of the plant.

Electrical connection of the plant electrode, or grounding electrode, to the plant contact point means that there is electrical connection between the plant electrode, or grounding electrode, and the plant contact point. In other words, this electrical connection of the plant electrode, or grounding electrode, with the plant contact point, allows flow of electrical current from the plant electrode, or the grounding electrode, to the plant contact point. Since, as mentioned above, there is no gap, and there is no air gap, between the plant electrode, or grounding electrode, and the plant contact point, there is also no electrical connection between the plant electrode, or the grounding electrode, and the plant contact point through a gap, specifically air gap, in between the plant electrode, or grounding electrode, and the plant contact point.

It should be emphasized, again, that the connection of the plant electrode, or the grounding electrode, with the plant contact point is mechanical as defined above and electrical as defined above.

It should be further noted that the growth medium contact point is not part of the stationary plant system, and is not under the scope of the present subject matter. A growth medium contact point is defined as a spot in a growth medium in which a plant grows, and is in contact with, to which a grounding electrode is mechanically and electrically connected.

Mechanical connection of the grounding electrode to the growth medium contact point means that there is a physical connection between the grounding electrode and the growth medium contact point. In some embodiments, there is no gap between the grounding electrode and the growth medium contact point. More specifically, there is no gap of air between the grounding electrode and the growth medium contact point. According to one embodiment, the physical connection between the grounding electrode and the growth medium contact point is robust. According to another embodiments, there is a need to invest force in order to separate the grounding electrode from the growth medium contact point.

In some embodiments, electrical connection of the grounding electrode to the growth medium contact point means that there is electrical connection between the grounding electrode and the growth medium contact point. In other words, this electrical connection of the grounding electrode with the growth medium contact point allows flow of electrical current from the grounding electrode to the growth medium contact point. Since, as mentioned above, there is no gap, and there is no air gap, between the grounding electrode and the growth medium contact point, there is also no electrical connection between the grounding electrode and the growth medium contact point through a gap, specifically air gap, in between the grounding electrode and the growth medium contact point.

It should be emphasized, again, that the connection of the grounding electrode with the growth medium contact point is mechanical as defined above and electrical as defined above.

According to one embodiment, the growth medium is electrically conductive. According to another embodiment, the growth medium is a liquid. According to another embodiment, the growth medium is soil. Thus, according to a further embodiment, the soil is electrically conductive. According to yet a further embodiment, the electrical conductivity of the soil can be changed. Any mechanism for changing the electrical conductivity of the soil is under the scope of the present subject matter, for example, by changing the fluid content of the soil, by changing the content of electrolytes, for example salt ions, in the soil, and the like.

According to another embodiment, the plurality of plants is a plurality of trees. According to yet another embodiment, the plant contact point is located above a bottom section of a trunk of the tree. According to yet another embodiment, the plant contact point is located above a midpoint of the trunk (substantially 50% of trunk's length). According to still another embodiment, the plant contact point is located above substantially 75% of the length of the trunk. According to a further embodiment, the plant contact point is located substantially at a first division line of the trunk, where the lowest limbs divide from the trunk.

According to a still another embodiment, the at least one power source comprises a plurality of plant electrodes, wherein each plant electrode is configured to mechanically and electrically connect to a plant contact point.

According to a further embodiment, the plurality of plant electrodes are configured to mechanically and electrically connect to a plurality of plant contact points that are spaced on a circumference of a plant.

According to yet a further embodiment, the plant electrode is configured to mechanically and electrically connect to at least one inner tissue, or layer that is below an epidermis layer of the plant.

According to still a further embodiment, the grounding electrode is configured to mechanically and electrically connect to at least one inner layer that is below an epidermis layer of the plant.

According to an additional embodiment, the plant electrode, or the grounding electrode, is configured to mechanically and electrically connect to a woody plant comprising a bark, and the at least one inner tissue, or layer is below the bark.

According to yet an additional embodiment, the plant electrode, or the grounding electrode is configured to mechanically and electrically connect to a hydroponic plant, when the roots of the hydroponic plant a positioned in a mist of liquid, or a sprayed liquid, or in a liquid. According to this embodiment, the plant electrode, or the grounding electrode, is configured to mechanically and electrically connect to at least one inner tissue, or layer that is below an epidermis layer of the roots of the hydroponic plant.

According to still an additional embodiment, the electrical potential of a part of the plant is affected by inducing an electrical current in the electrical circuit.

According to another embodiment, an anode of the power source is electrically connected to the plant electrode, and a cathode of the power source is electrically connected to the grounding electrode, causing the plant to be negatively charged. In this embodiment, the plant is negatively charged when the plant electrode is electrically and mechanically connected to the plant, and the grounding electrode is electrically and mechanically connected either to the plant, or to growth medium in which the plant grows and in contact with.

According to yet another embodiment, a cathode of the power source is electrically connected to the plant electrode, and an anode of the power source is electrically connected to the grounding electrode, causing the plant to be positively charged. In this embodiment, the plant is positively charged when the plant electrode is electrically and mechanically connected to the plant, and the grounding electrode is electrically and mechanically connected either to the plant, or to growth medium in which the plant grows and in contact with.

According to still another embodiment, the power source is a direct current (DC) power source. According to an additional embodiment, the power source is an alternating current (AC) power source. According to yet an additional embodiment, the AC power source provides an alternating electrical current in a frequency of at least substantially 0.1 Hz. According to still an additional embodiment, the AC power source provides an alternating electrical current in a frequency of substantially less than 0.1, 1, 10, 100, 1,000, 10⁴, 10⁵, 10⁶, 10⁷, 10⁸, 10⁹, 10¹⁰, 10¹¹, or 10¹² Hz, and the like.

According to a further embodiment, in the stationary plant system the at least one plant electrode is in a form of a plant penetrating element that is configured to electrically connect to the power source, and to conduct an electrical current to at least one inner tissue, or layer of the plant, wherein the first contact point is in at least one inner tissue, or layer of the plant. Embodiments of the plant electrode in a form of a plant penetrating element are described hereinafter. According to yet a further embodiment, in the electrical circuit that is formed by the stationary plant system the plurality of plants is electrically connected in parallel. According to an additional embodiment, in the electrical circuit that is formed by the stationary plant system the plurality of plants is electrically connected in series.

According to yet an additional embodiment, the electrical circuit that is formed by the stationary plant system comprising: a closed sub-circuit in which an electrical current can flow through a part of the plant and through components of the stationary plant system; and an open sub-circuit, at another part of the plant in which an electrical current cannot flow, and an electrical potential can be formed.

According to still an additional embodiment, the stationary plant system is configured to affect the electrical potential in the open sub-circuit by manipulating the electrical current that flows through the closed sub-circuit.

According to another embodiment, the stationary plant system is configured to either monitor, or control, or monitor and control the electrical potential of the plurality of plants; or monitor, or control, or monitor and control the electrical current that flows through the plurality of plants; or monitor, or control, or monitor and control contact of particles with a part of the plant, or any combination thereof.

The present subject matter further provides a method for manipulating an electrical potential of a plurality of plants by forming an electrical circuit in which electrical current can flow upon desire through the plurality of plants, the method comprising: providing a stationary plant system; electrically connecting at least one power source to a plurality of plant electrodes and to at least one grounding electrode; mechanically and electrically connecting each plant electrode to a plant contact point in an inner tissue, or layer of the plant; mechanically and electrically connecting each grounding electrode to a growth medium contact point that is in a growth medium in which the plurality of plants grow and that the plurality of plants are in contact with the growth medium, thereby forming an electrical circuit between the at least one power source, the plurality of plant contact points and the at least one growth medium contact point, inducing an electrical current that can flow through the electrical circuit, thereby affecting the electrical potential of at least part of the plurality of plants. In other words, the method for manipulating an electrical potential of a plurality of plants by forming an electrical circuit in which electrical current flows upon desire through the plurality of plant uses the stationary plant system described herein.

According to one embodiment, the stationary plant system is for affecting movement of electrically charged particles to, from, or within, the plurality of plants. In other words, the stationary plant system is configured to affect movement of electrically charged particles to, from, or within, the plurality of plants.

According to one embodiment, in the electrical circuit that is formed by the stationary plant system, electrical current flows from the power source, to the plurality of plant electrodes, from each plant electrode to a plant contact point, through at least a part of the plurality of plant, through roots of the plurality of plants-, with a return path through the growth medium to the at least one growth medium contact point, from each growth medium contact point to a grounding electrode and back to the power source.

According to one embodiment, the stationary plant system is configured to manipulate an electrical potential of a plurality of plants, between the plurality of plant electrodes and the at least one grounding electrode.

According to one embodiment, the stationary plant system is configured to manipulate the electrical potential of one plant. According to one embodiment, the stationary plant system is for manipulating an electrical potential of at least one plant, of parts of the plant that are not part of the electrical circuit.

According to one embodiment, the stationary plant system is configured to form an electrical circuit, wherein the electrical potential of the plurality of plants, or at least one part of the plurality of plants, is affected by applying an electrical potential between the plurality of plant electrodes and the at least one grounding electrode.

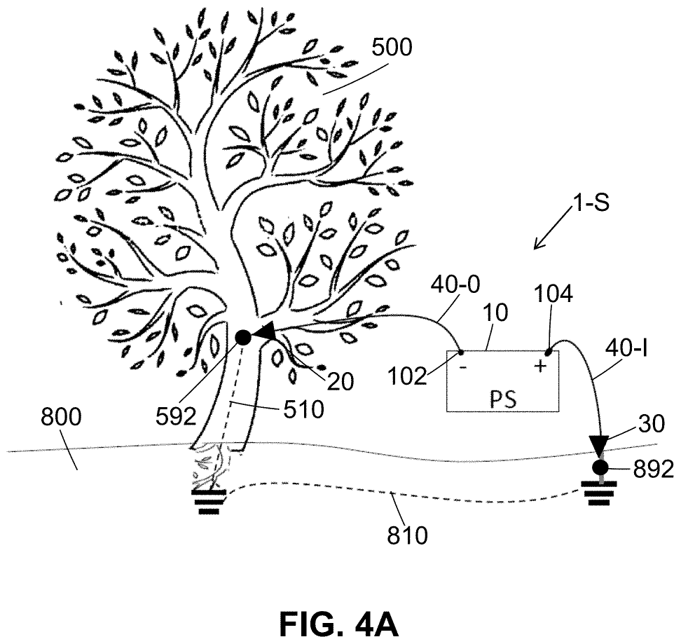

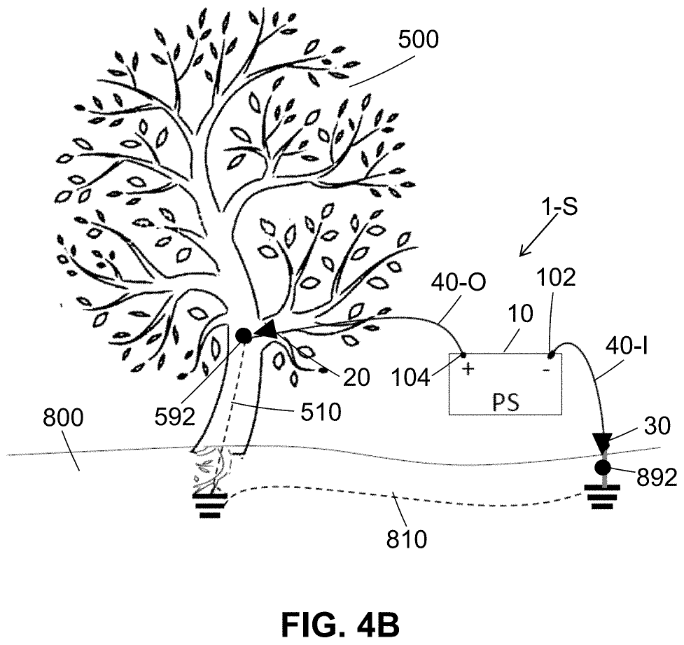

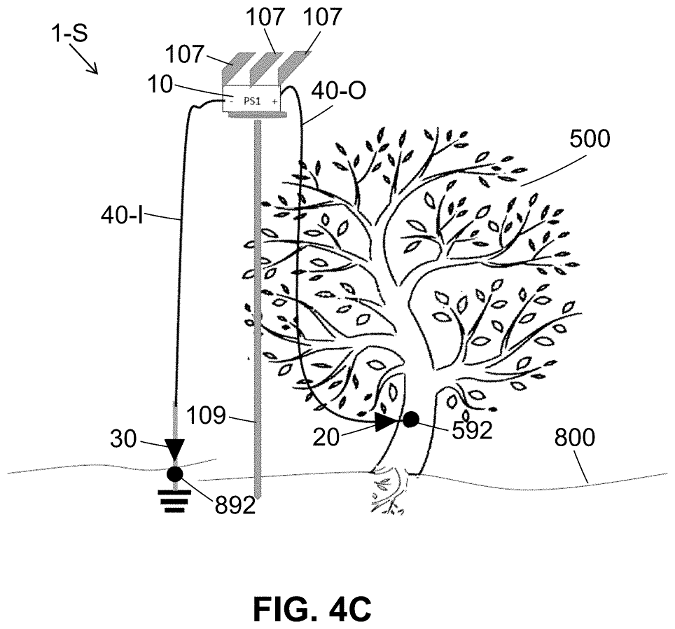

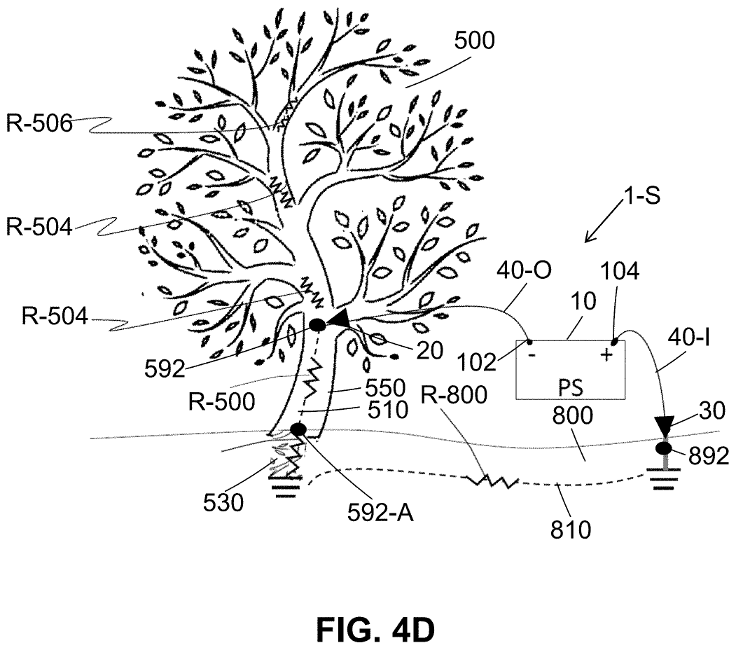

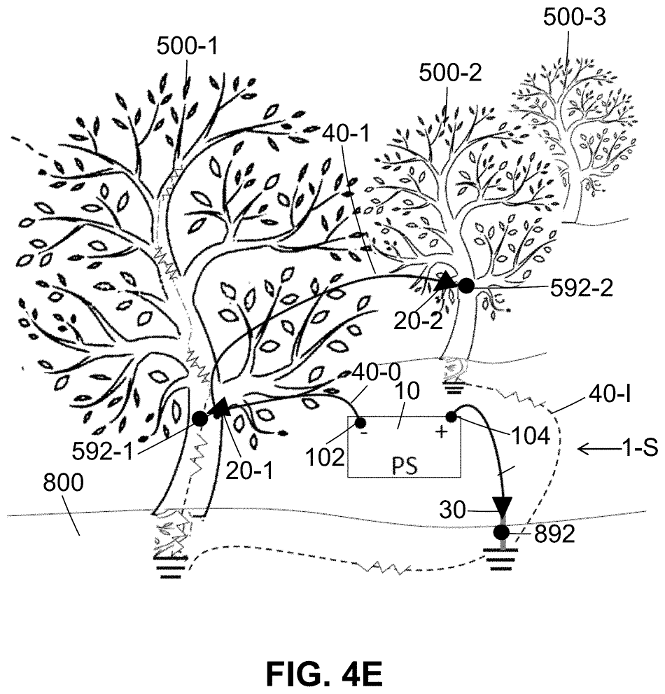

Referring now to FIGS. 4A-4E, schematically illustrating, according to some embodiments, a side view of a stationary plant system comprising a power source mechanically and electrically connected to at least one plant and to a growth medium. FIGS. 4A-D show a stationary plant system 1-S that is configured to mechanically and electrically connect to one plant 500, and FIG. 4E shows a stationary plant system 1-S that is configured to mechanically and electrically connect to a plurality of plants 500. FIGS. 4A-E illustrate the basic components of the stationary plant system 1-S and their connection with the at least one plant 500 and the growth medium 800.

FIG. 4A shows a plant 500, for example a tree 500, planted in a growth medium 800, for example soil 800, and the stationary plant system 1-S mechanically and electrically connected to the plant 500 and the growth medium 800. The stationary plant system 1-S illustrated in FIG. 4A comprises a power source 10 electrically connected to a plant electrode 20 and a grounding electrode 30. The plant electrode 20 is mechanically and electrically connected to a plant contact point 592 at the plant 500, and the grounding electrode 30 is mechanically and electrically connected to a growth medium contact point 892 at the growth medium 800. According to the embodiment shown in FIG. 4A, the growth medium 800 is soil 800 in which the plant 500 grows and with which the plant 500 is in contact.

According to one embodiment, the stationary plant system 1-S is connected to the at least one plant 500, and alternatively to the growth medium 800 with at least one electrically conductive cable 40. Various types of the electrically conductive cable 40 are described hereinafter. Any element that is electrically conductive can serve as an electrically conductive cable 40, for example an electricity cable 40, a rod 40 made of an electricity conductive material, a chain 40 made of an electricity conductive material, and the like.

The electrically conductive cable 40 that electrically connects the power source 10 to the plant electrode 20 is termed hereinafter “outward electrically conductive cable 40-O”, and is configured to conduct an electrical current from the power source 10 to the plant electrode 20. The electrically conductive cable 40 that electrically connects the grounding electrode 30 to the power source 10 is termed hereinafter “inward electrically conductive cable 404”, and is configured to conduct an electrical current from the grounding electrode 30 to the power source 10.

According to one embodiment, shown in FIG. 4A, the outward electrically conducting element 40-O is electrically connected to an anode 102 (negative electrode) of the power source 10, and the inward electrically conducting element 40-I is electrically connected to a cathode 104 (positive electrode) of the power source 10. As a result, when an electrical circuit is formed in the setting shown in FIG. 4A, the plant 500 is negatively charged. In other words, according to this embodiment, an anode 102 of the power source 10 is electrically connected to the plant electrode 20 that is electrically and mechanically connected to the plant contact point 592, and a cathode 104 of the power source 10 is electrically connected to the grounding electrode 30 that is electrically and mechanically connected to the growth medium contact point 892, causing the plant 500 to be negatively charged.

Referring now to FIG. 4B. According to another embodiment, shown in FIG. 4B, the outward electrically conducting element 40-O is electrically connected to a cathode 104 (positive electrode) of the power source 10, and the inward electrically conducting element 40-I is electrically connected to an anode 102 (negative electrode) of the power source 10. As a result, when an electrical circuit is formed in the setting shown in FIG. 4B, the plant 500 is positively charged. In other words, according to this embodiment, a cathode 104 of the power source 10 is electrically connected to the plant electrode 20 that is electrically and mechanically connected to the plant contact point 592, and an anode 102 of the power source 10 is electrically connected to the grounding electrode 30 that is electrically and mechanically connected to the growth medium contact point 892, causing the plant 500 to be positively charged.

In the drawings hereinafter, the outward electrically conducting element 40-O is electrically connected to an anode 102 (negative electrode) of the power source 10, and the inward electrically conducting element 40-I is electrically connected to a cathode 104 (positive electrode) of the power source 10, as shown in FIG. 4A. It should be noted that this embodiment is given as an example only and should not be considered as limiting the scope of the present subject matter. An opposite orientation of connection of the outward electrically conducting element 40-O and the inward electrically conducting element 40-I, as shown in FIG. 4B, can also be established in the embodiments shown in shown in the following drawings.

These two embodiments demonstrate the ability of the stationary plant system 1-S of the present subject matter to control, and change, inter alia, the electrical polarity of the at least one plant 500 that is part of the electrical circuit formed by the stationary plant system 1, by connecting the outward electrically conducting element 40-O and the inward electrically conducting element 40-I to desired electrodes of the power source 10, as described above.

As further shown in FIGS. 4A-B, an electrical circuit is formed. The electrical circuit comprises the power source 10, an outward electrically conductive cable 40-O connecting the power source 10 to the plant electrode 20 that is electrically and mechanically connected to the plant contact point 592 at the plant 500, the plant 500, the growth medium 800, the grounding electrode 30 that is electrically and mechanically connected to the growth medium contact point 892 at the growth medium 800, and an inward electrically conductive cable 40-I connecting the grounding electrode 30 to the power source 10. The part of the electrical circuit that is in the plant 500 is depicted with dashed line 510, and the part of the electrical circuit that is in the growth medium 800 is depicted with dashed line 810. A closed electrical circuit allows manipulation of the electrical potential of the plant 500.

According to one embodiment, the grounding electrode 30 is electrically and mechanically connected to a soil contact point 892 in the growth medium 800. According to another embodiment, the grounding electrode 30 is electrically and mechanically connected to a soil contact point 892 in the growth medium 800 in close proximity to the plant 500, for example at a distance of up to substantially 1 cm, or substantially 5 cm, or substantially 10 cm, or substantially 50 cm, and the like. According to yet another embodiment, the grounding electrode 30 is electrically and mechanically connected to a soil contact point 892 in the growth medium 800 adjacent to the plant 500, for example up to substantially 1 meter, or substantially 2 meters, or substantially 3 meters, or substantially 4 meters, or substantially 5 meters, and the like. According to still another embodiment, the grounding electrode 30 is electrically and mechanically connected to a soil contact point 892 in the growth medium 800 in a distance from the plant 500, for example up to substantially 10 meters, substantially 50 meters, substantially 100 meters, substantially 200 meters, substantially 500 meters, substantially 1,000 meters, and the like.

Thus, when the grounding electrode 30 is electrically and mechanically connected to a soil contact point 892 in the growth medium 800, the electrical circuit comprises the power source 10, the at least one plant 500, and the growth medium 800. This embodiment of the stationary plant system 1-S is configured to manipulate an electrical potential difference between the at least one plant 500, or between the at least one part of the at least one plant 500, and the growth medium 800 in which the at least one plant 500 is planted, when the growth medium 800 is part of the electrical circuit, and in one embodiment, the electrical current returns to the power source 10 through the growth medium 800.

The power source 10 is of any known type of power source 10. Some types of power source 10 include: a chemical power storage device, for example a battery; a capacitor; an inductor, a solar power source configured to transform solar energy to electrical power, for example, at least one solar panel, as shown hereinafter in FIG. 4C; and a kinetic generator configured to transform kinetic energy, for example wind energy, water flow energy, kinetic energy of a motor and the like, to electrical power.

Referring now to FIG. 4C, schematically illustrating a side view of a stationary plant system comprising a power source comprising at least one solar panel according to some embodiments of the invention. The stationary plant system 1-S shown in FIG. 4C is similar to the stationary plant system 1-S shown in FIGS. 4A-B. However, according to the embodiment shown in FIG. 4C, the power source 10 further comprises at least one solar panel 107 configured to transform solar energy to electrical power. In order to increase the effectiveness of the at least one solar panel 107, there is an option to elevate at least the at least one solar panel 107, or the at least one solar panel 107 with the power source 10, so the at least one solar panel 107 would not be held in the shade of the plants 500, for example trees 500. By elevating the at least one solar panel 107 above the plants 500, the at least one solar panel 107 is exposed continuously to sun light during day times and not shaded by the plants 500. This can be achieved, for example, by placing at least the at least one solar panel 107, or the power source 10 with the at least one solar panel 107, on a power source stand 109 that is configured to hold at least the at least one solar panel 107, or the power source 10 with the at least one solar panel 107. Thus, according to another embodiment, the stationary plant system 1-S comprises at least one power source stand 109 that is configured to hold at least the at least one solar panel 107, or the power source 10 with the at least one solar panel 107, above the plants 500. According to one embodiment, the power source stand 109 is embedded in the growth medium 800, as shown in FIG. 4C. According to another embodiment, the power source stand 109 is attached to a plant 500.

According to one embodiment, the stationary plant system 1-S illustrated in FIG. 4C further comprises an electrical energy storing element electrically connected to at least one solar panel 107, and configured to be electrically charged by the at least one solar panel 107. In this sense, the power source 10 shown in FIG. 4C can serve as an electrical energy storing element 10. Thus, the electrical charging of the electrically energy storing element 10, by the at least one solar panel 107 can be performed during daytime and clear sky, when solar energy can reach the at least one solar panel 107. When solar energy is not available, for example during the night, or during cloudy days, for example, conduction of electrical current through the plants 500 is still possible, by using the electrical energy stored in the electrical energy storing element 10.

According to one embodiment, the power source 10 is a direct current (DC) power source 10. According to another embodiment, the DC power source 10 is a high voltage DC power source 10, generating, for example, more than substantially 1,500 Volts. According to yet another embodiment, the DC power source 10 is a low voltage DC power source 10, generating, for example, a range of substantially 120 to 1,500 Volts. According to still another embodiment, the DC power source 10 is an extra-low voltage DC power source 10, generating, for example, less than substantially 120 Volts.

According to one embodiment, the power source 10 is an alternating current (AC) power source 10. According to another embodiment, the AC power source 10 is a high voltage AC power source 10, generating, for example, more than substantially 1,000 Volts. According to yet another embodiment, the AC power source 10 is a low voltage AC power source 10, generating, for example, a range of substantially 50 to 1,000 Volts. According to still another embodiment, the AC power source 10 is an extra-low voltage AC power source 10, generating, for example, less than substantially 50 Volts. According to one embodiment, the DC power source 10 is configured to supply an AC current carried on a DC current.

Referring now to FIG. 4D schematically illustrating a side view of some electrical resistance points in a plant and a growth medium mechanically and electrically connected to a stationary plant system according to some embodiments of the invention. FIG. 4D is essentially similar to FIG. 4A. However, FIG. 4D additionally shows some electrical resistance points in the plant 500 and the growth medium 800 that are electrically and mechanically connected to the stationary plant system 1-S. In the electrical circuit that is formed, the growth medium 800 and parts of the plant 500 can have a certain level of resistance to the electrical current, that depends on the location where the electrical resistance is measured. The electrical resistance at a point in the growth medium 800 is termed “growth medium resistance R-800”. The electrical resistance at a point in a stem of a plant 500, or a trunk of a tree 500, is termed “plant resistance R-500”. The electrical resistance at a point in a branch of the plant 500 is termed “branch resistance R-504”. The electrical resistance at a point in a twig of the plant 500 is termed “twig resistance R-506”. Generally, as the electrical resistance point in a part of the plant 500 is closer to the growth medium 800, the electrical resistance at that point is lower. Accordingly, the plant resistance R-500 is lower than the branch resistance R-504; and the branch resistance R-504 is lower than the twig resistance R-506.

According to one embodiment, shown for example in FIG. 4D, the electrical circuit that is formed by the stationary plant system 1-S comprises a closed sub-circuit in which there is a flow of an electrical current, depicted with dashed line 510, through a part of the at least one plant 500 and through components of the stationary plant system 1-S; and an open sub-circuit, at another part of the at least one plant 500 in which there is no flow of electrical current, and there is formation of an electrical potential.

According to a further embodiment, the closed sub-circuit comprises components of the stationary plant system 1, a part of the plant 500, and in some embodiments, like the embodiment shown in FIG. 4D, also the growth medium 800. For example, in the embodiment shown in FIG. 4D, the closed sub-circuit runs in the following path: anode 102 of the power source 10—outward electrically conductive cable 40-O—plant electrode 20 electrically and mechanically connected to a plant contact point 592 at a plant 500—through part of the plant 510 between the plant contact point 592 and the growth medium 800—through the growth medium 810—growth medium contact point 892 at the growth medium 800—grounding electrode 30 electrically and mechanically connected to the growth medium contact point 892—inward electrically conductive cable 40-I—cathode 104 of the power source 10. This example shows the nature of the closed sub-circuit, that comprises parts of the stationary plant system 1-S, a part of the plant 500 and the growth medium 800. According to an additional embodiment, the part of the plant 500 that is part of the closed sub-circuit is between the plant contact point 592 and the growth medium 800. In other words, the part of the plant 500 that is part of the closed sub-circuit is the part of the plant 500 that is below the plant contact point 592, to which the plant electrode 20 is electrically and mechanically connected.

According to yet a further embodiment, the open sub-circuit comprises parts of the plant 500 that are not part of the closed sub-circuit, and there is no flow of an electrical current through these parts of the plant, but there is formation of an electrical potential in these parts of the plant 500. According to this embodiment, parts of the plant 500 that are above the plant contact point 592, to which the plant electrode 20 is electrically and mechanically connected, are parts of the open sub-circuit.

According to one embodiment, as can be understood from the aforementioned description, the position of the plant contact point 592 at the plant 500, to which the plant electrode 20 is electrically and mechanically connected, determines which parts of the plant 500 are parts of the closed sub-circuit, and which parts of the plant 500 are parts of the open sub-circuit. In other words, the position of the plant contact point 592, at the plant 500, to which the plant electrode 20 is electrically and mechanically connected, determines through which parts of the plant 500 there is a flow of an electrical current. Thus, according to the embodiment shown in FIG. 4D, the plant electrode 20 is electrically and mechanically connected to a plant contact point 592 that is positioned at an upper edge of the trunk 550, in a trunk's 550 first division line, of a tree/plant 500. As a result, the entire trunk 550 is part of the closed sub-circuit, whereas any part of the tree 500 that is above the plant contact point 592 at the trunk 550 is part of the open sub-circuit. When, for example, it is desired to include also the trunk 550 in the open sub-circuit, it is possible by electrically and mechanically connecting the plant electrode 20 to an alternative plant contact point 592-A at the lowest edge of the trunk 550, close to the growth medium 800. As a result, only roots 530 of the plant 500, or tree 500, are part of the closed sub-circuit, whereas the trunk 510, and upper parts of the plant 500 that are above the alternative plant contact point 592-A are part of the open sub-circuit. When, for example, it is desired to include also a portion of the trunk 550 in the open sub-circuit, this can be achieved by electrically and mechanically connecting the plant electrode 20 to an alternative plant contact point 592-A at a lower position on the trunk 550, for example close to the growth medium 800.

In some embodiments, manipulating the plant electrical potential may be conducted to increase the provision of nutritive and fertilizing materials to the plants roots. Therefore, the location of contact point 592 at the plant 500 may be determined in order to optimize the nutritive flow towards roots 530 and further from roots 530 to different parts of plant 500.

According to one embodiment, both the closed sub-circuit and the open sub-circuit are affected by the voltage of the power source 10, and by the nature of the components of the stationary plant system 1-S, for example the electrical resistance of the various types of the electrically conductive cable 40. Accordingly, the voltage that is formed in the electrical circuit corresponds to the type (DC or AC), polarity and voltage level of the power source 10.

It should be emphasized again that the closed sub-circuit is formed due to the electrical and mechanical connection of components of the stationary plant system 1-S with the plant 500 and the growth medium 800, and that the part of the plant 500 and the growth medium 800, which are part of the formed closed sub-circuit, are not part of the present subject matter. However, the components of the stationary plant system 1-S that cause the formation of the closed sub-circuit in particular, and the electrical circuit in general, are parts of the present subject matter.

FIG. 4E shows a plurality of plants 500, for example a plurality of trees 500, planted in a growth medium 800, for example soil 800, and the stationary plant system 1-S mechanically and electrically connected to the plurality of plants 500 and the growth medium 800. FIG. 4E shows a number of three plants 500, designated first plant 500-1, second plant 500-2 and third plant 500-3, of which the first plant 500-1 and the second plant 500-2 (totally two plants) are electrically and mechanically connected to the stationary plant system 1-S. It should be noted though that a number of two plants 500 that are electrically and mechanically connected to the stationary plant system 1-S is given as an example only. The stationary plant system 1-S is configured to electrically and mechanically connect to any number of plants 500.

The stationary plant system 1-S illustrated in FIG. 4E comprises a power source 10 electrically connected to a plurality of plant electrodes 20 and to a grounding electrode 30. In the embodiment shown in FIG. 4E, the stationary plant system 1-S comprises two plant electrodes 20—plant electrode No. 1 20-1 and plant electrode No. 2 20-2. It should be noted, though, that a number of two plant electrodes 20 of the stationary plant system 1-S is given as an example only. The stationary plant system 1-S can comprise any number of plant electrodes 20.

In addition, the stationary plant system 1-S shown in FIG. 4E comprise one grounding electrode 30. Nevertheless, it should be noted that an embodiment of the stationary plant system 1-S comprising one grounding electrode 30 is given as an example only. The stationary plant system 1-S can comprise any number of grounding electrodes 30. According to one embodiment, the number of grounding electrodes 30 is lower than the number of plant electrodes 20. Thus, the stationary plant system 1-S shown in FIG. 4E comprises two plant electrodes 20 and one grounding electrode 30.

According to one embodiment, each plant electrode 20 is mechanically and electrically connected to a plant contact point 592 at different plants 500. Thus, as illustrated in FIG. 4E, the plant electrode No. 1 20-1 is electrically connected to a first plant contact point 592-1 in the first plant 500-1, and the plant electrode No. 2 20-2 is electrically connected to a second plant contact point 592-2 in the second plant 500-2.

In addition, the grounding electrode 30 is mechanically and electrically connected to a growth medium contact point 892 at the growth medium 800. Also, according to the embodiment shown in FIG. 4B, the growth medium 800 is soil 800 in which the plurality of plants 500 grow and with which the plurality of plants 500 are in electrical and mechanical contact.

Regarding the electrically conductive cables 40 shown in FIG. 4E, according to the embodiment shown in FIG. 4E, the stationary plant system 1-S comprises an outward electrically conductive cable 40-O between the anode 102 of the power source 10 and the plant electrode No. 1 20-1, a first electrically conductive cable 40-1 between the plant electrode No. 1 20-1 and the plant electrode No. 2 20-2, and an outward electrically conductive cable 40-O between the cathode 104 of the power source 10 and the grounding electrode 30.

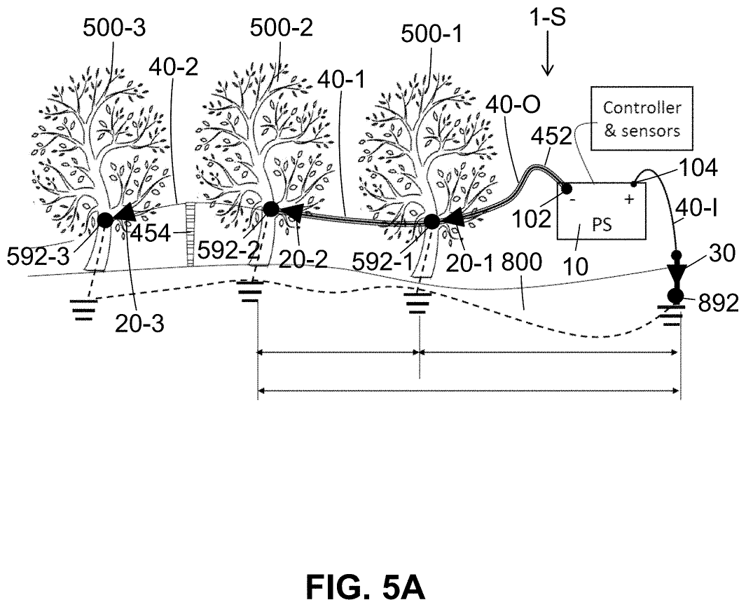

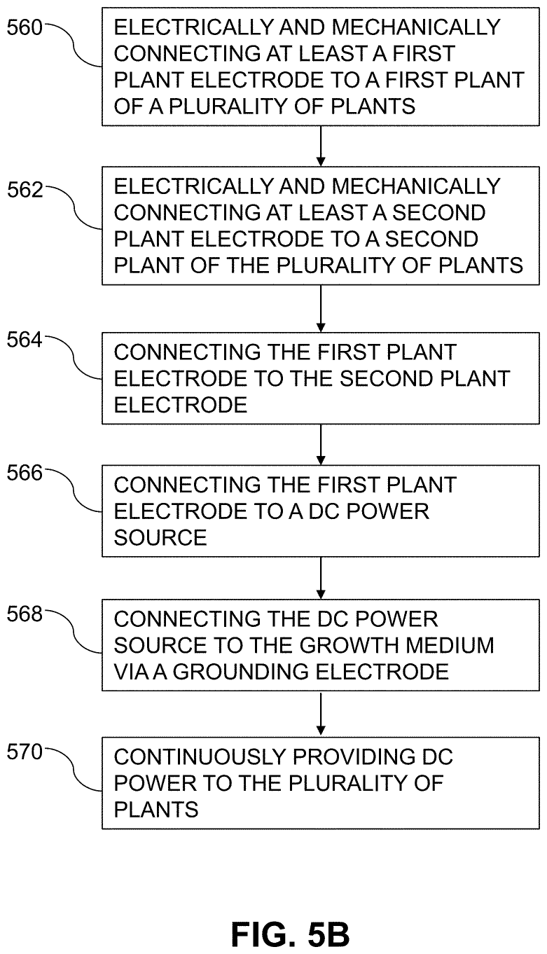

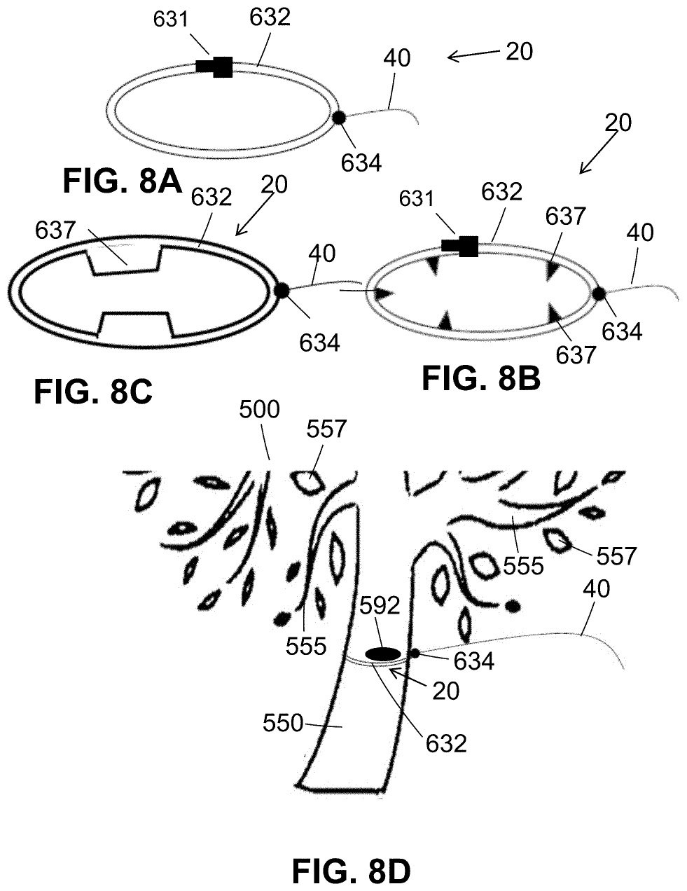

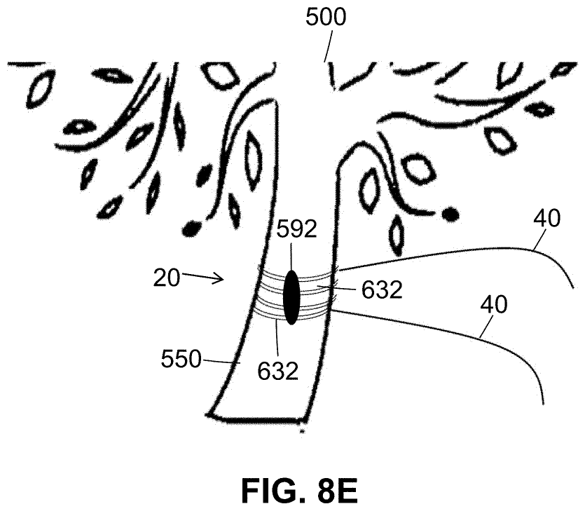

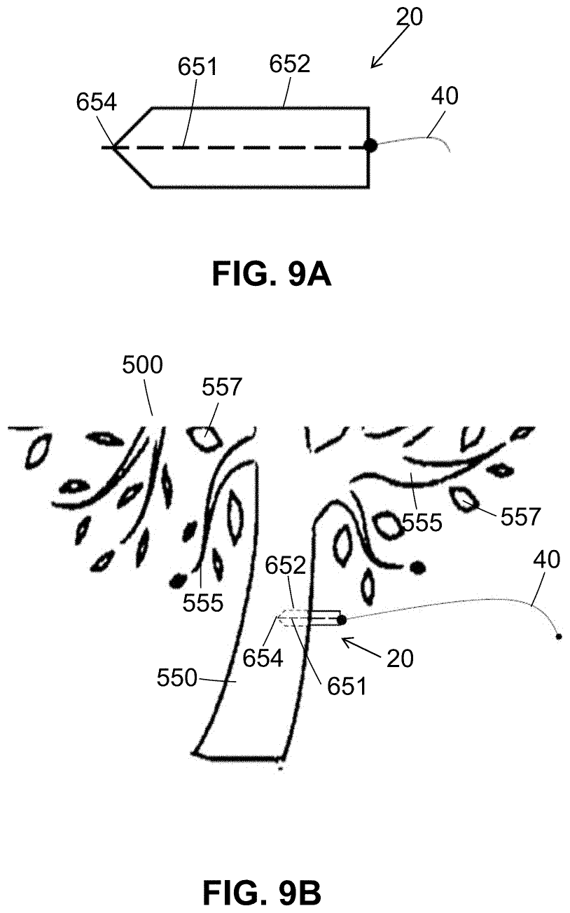

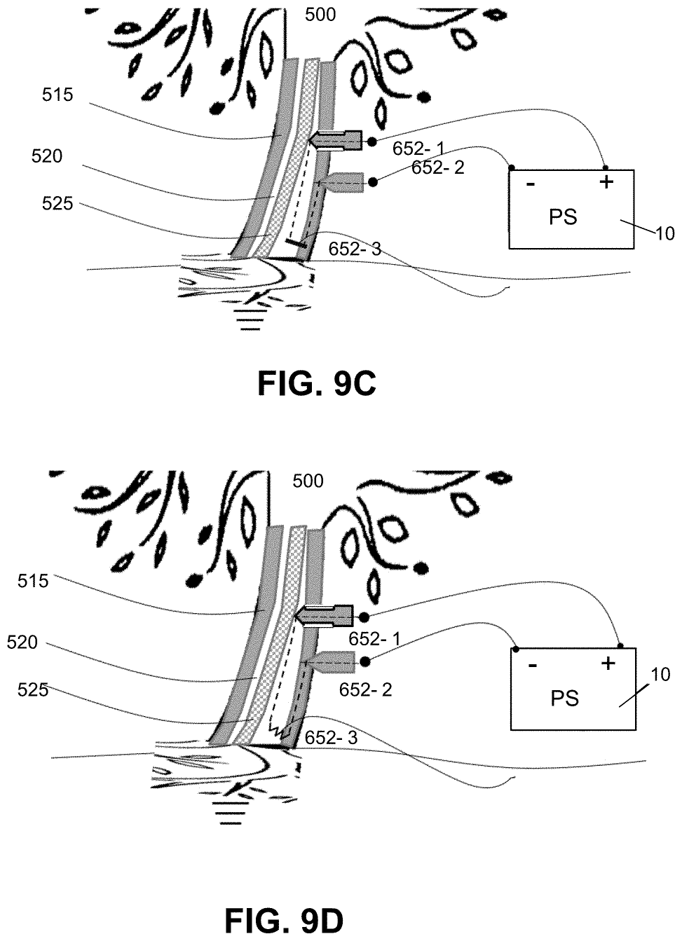

Referring now to FIG. 5A, schematically illustrating some additional embodiments of a plant stationary system and its connection to a plurality of plants. In some embodiments, system 1-S is configured to manipulate an electrical potential of plants. FIG. 5A shows a plant stationary system 1-S electrically and mechanically connected to a plurality of plants 500, for example a plurality of trees 500—a first plant 500-1, a second plant 500-2 and a third plant 500-3. A cathode 104 of the power source 10 is electrically connected to a grounding electrode 30, and the grounding electrode 30 is electrically and mechanically connected to a growth medium contact point 892 in a growth medium 800 in which the plurality of plant 500 grows, or planted, and with which the plurality of plants 500 is in contact. An anode 102 of the power source 10 is electrically connected to a plant electrode No. 1 20-1, which is electrically and mechanically connected to a first plant contact point 592-1 at the first plant 500-1. System 1-S comprises two or more plant electrodes, for example, 20-1 to 20-3, each electrically and mechanically connectable to a plant 500-1 to 500-3, wherein at least a portion of each plant electrode is insertable into inner layers of the plant, as illustrated and discussed with respect to FIGS. 8A-8E and 9A-9D. The plant electrode No. 1 20-1 is electrically connected to a plant electrode No. 2 20-2 with a first electrically conductive cable 40-1. The plant electrode No. 2 20-2 is electrically and mechanically connected to a second plant contact point 592-2 at the second plant 500-2. The plant electrode No. 2 20-2 is also electrically connected to a plant electrode No. 3 20-3 with a second electrically conductive cable 40-2. In addition, the plant electrode No. 3 20-3 is electrically and mechanically connected to a third plant contact point 592-3 at the third plant 500-3. The connection of the stationary plant system 1-S with the plurality of plants 500 and the growth medium 800 shown in FIG. 5 gives rise to a closed electrical circuit that includes the aforementioned components of the stationary plant system 1-S, and the plurality of plants 500 and growth medium 800 that are electrically and mechanically connected to the stationary plant system 1-S.

According to one embodiment, shown in FIG. 5A, most of the electrically conductive cables 40 of the stationary plant system 1-S are electrically insulated from the growth medium 800 (e.g., soil). In this sense, the growth medium 800 is considered also an electrical ground 800. Any type of electrical insulation of the electrically conductive cables 40 from the growth medium 800 is under the scope of the present subject matter, of which some embodiments are shown in FIG. 5.

According to one embodiment, the electrically conductive cable 40 is coated with an electrical insulating material. For example, the outward electrically conductive cable 40-O and the first electrically conductive cable 40-1, shown in FIG. 5, are electrically insulated with an electrical insulating coating 452 that covers at least some of a length of the outward electrically conductive cable 40-O and the first electrically conductive cable 40-1, in a manner that prevents electrical connection between the outward electrically conductive cable 40-O and the first electrically conductive cable 40-1 with the growth medium 800. The electrical insulating coating is made of any electrical insulating material, for example plastic, rubber, silicon, wood and the like.

According to another embodiment, the electrically conductive cable 40 is electrically insulated from the growth medium 800 by supporting the electrically conductive cable 40 with an electrically insulated support 454, as shown in FIG. 5A, in which a second electrically conductive cable 40-2 is supported with an electrically insulated support 454. This embodiment is relevant especially, but not only, to electrically conductive cables 40 that are long and there is a possibility that they may bend, or become loose, and electrically connect to the growth medium 800. Any type of electrically insulated support 454 is under the scope of the present subject matter, for example but not limited to, a pole made of, or coated with, an electrical insulating material, like a pole made of wood or plastic, or a pole coated with a plastic, rubber, or silicon cover, and the like. In this regard, it has been found by the inventor that also a pole made of iron, which is electrically conductive, that is covered with rust, can serve as an electrically insulated support 454, since rust is an electrical insulator. Another mechanism for electrically insulating an electrically conductive cable 40 from the growth medium 800 is by fixing the electrically conductive cable 40 to the plant 500, and using the plant 500, for example a tree 500, as an electrically insulated support 454. Yet another mechanism can be fixing the electrically conductive cable 40 at a high altitude above the growth medium 800, in a manner that minimizes the chance for the electrically conductive cable 40 to touch the growth medium 800.

In some embodiments, the electrical insulation of the electrically conductive cables 40 from the growth medium 800 is important for the function of the stationary plant system 1-S, and specifically for the manipulation of the electrical potential of the plurality of plants 500. When an electrically conductive cable 40 electrically connects to the growth medium 800, an electrical short circuit can be formed between the power source 10 and the growth medium 800, thus eliminating at least one plant 500 from the electrical circuit that is formed by the stationary plant system 1-S, and as a result preventing manipulation of the electrical potential of this at least one plant 500.

According to one embodiment, the stationary plant system 1-S is for manipulating the electrical potential of a plurality of plants 500, namely more than one plant 500, or more than substantially 10, 100, 1,000, 10⁴, 10⁵, 10⁶, 10⁷, 10⁸, or more plants 500.

According to one embodiment, when the electrical potential of a plurality of plants 500 is manipulated, the plurality of plants 500 is electrically connected in parallel, for example trees in an orchard, trees in a portion of an orchard, a group of shrubs, a flower bed, and the like.

Referring now to FIG. 5B which is a flowchart of a method for manipulating an electrical potential of a plurality of plants according to some embodiments of the invention. The method of FIG. 5B may be performed using system 1-S according to any embodiment of the invention. In step 560, at least a first plant electrode may be electrically and mechanically connected to a first plant of a plurality of plants. For example, a first plant electrode 20-1 may be electrically and mechanically connected to plant 500-1 (e.g., tree 500-1 illustrated). In some embodiments, electrically and mechanically connecting first plant electrode 20-1 to the first plant 500-1 may include inserting at least a portion of each plant electrode into inner layers of the plant, in proximity to a lowest branching point of each plant, for example, point 592-1. Some nonlimiting examples for inserting at least a portion of each plant electrode into inner layers of the plant are discussed with respect to FIGS. 8A-8E and 9A-9B.

The inventors surprisingly found the location of first contact point along the trunk length has a significant effect on the electrical potential measured in the crown of the tree. The distance from the soil results in large differences in electrical potential, as measured in reference to the electrical ground.

In step 562, at least a second plant electrode may be electrically and mechanically connected to a second plant of a plurality of plants. For example, a second plant electrode 20-2 may be electrically and mechanically connected to plant 500-2 (e.g., tree 500-2 illustrated). In some embodiments, plant 500-2 is not a neighboring plant of plant 500-1. In some embodiments, electrically and mechanically connecting second plant electrode 20-2 to the second plant 500-2 may include inserting at least a portion of each plant electrode into inner layers of the plant, in proximity to a lowest branching point of each plant, for example, point 592-2. Some nonlimiting examples for inserting at least a portion of each plant electrode into inner layers of the plant are discussed with respect to FIGS. 8A-8E and 9A-9B.

In some embodiments, inserting the at least a portion of each plant electrode to each plant is at a height of at least 50% of the height of the lowest branching point from the growth medium. In some embodiments, inserting the at least a portion of each plant electrode to each plant is at a height of at least 75% of the height of the lowest branching point from the growth medium. In some embodiments, inserting the at least a portion of each plant electrode to each plant is at a height of at least 90% of the height of the lowest branching point from the growth medium. Therefore, point 592-1 and point 592-2 may be located at a height of at least, 50%, 75% 90% or more of the height of the lowest branching point from the surface of growth medium 800.

In some embodiments, steps 560 and 562 may be repeated for additional plants, for example, trees in a multiple orchard, at least some rows of trees in an orchard, multiple grapevines in a vinery, multiple plants in a field and the like. In step 564, the first plant electrode may be connected to the second plant electrode. For example, first plant electrode 20-1 may be connected to second plant electrode 20-2 via electrically conductive cable 40-1 isolated from growth medium 800, as illustrated in FIG. 5A.

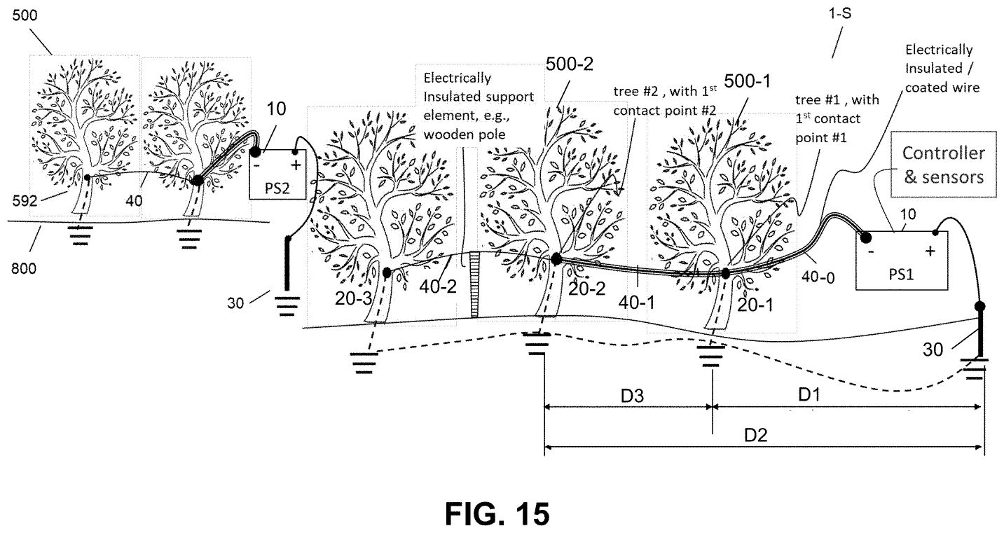

In some embodiments, step 564 may be repeated for additional plants. For example, second plant electrode 20-2 may be connected to third plant electrode 20-3 via electrically conductive cable 40-2. In some embodiments, first electrically conductive cable 40-1 differs from second electrically conductive cable 40-2 by at least one of, thickness, length, and conductivity, as illustrated and discussed with respect to FIG. 15. In some embodiments, the diameter of cables 40-1 and 40-2 may be determined such that the cables ohmic resistance is smaller than, 10%, 5%, 2%, 1% or less of the ohmic resistance of growth medium 800.

In step 566, the first plant electrode may be connected to a DC power source. For example, first plant electrode 20-1 may be connected to DC power source 10 via an electrically conductive cable 40-O isolated from growth medium 800, as illustrated in FIG. 5A.

In step 568, the DC power source may be connected to the growth medium via a grounding electrode. For example, DC power source 10 may be connected to growth medium 800 via grounding electrode 30. In some embodiments, grounding electrode 30 is located at least 5 meters from at least one plant electrode. For example, grounding electrode 30 may be located at least 5 meters from first plant electrode 20-1, second plant electrode 20-2, third plant electrode 20-3, etc. In some embodiments, grounding electrode 30 may be located at least, 2 m, 4 m, 5 m, 6 m, 7, 10 m, 20 m or more from first plant electrode 20-1.

In step 570, DC power may continuously be provided to the plurality of plants. For example, DC power source 10 may remain electrically connected to first plant electrode 20-1 for at least 2 minutes. In some embodiments, DC power source 10 may remain electrically connected to first plant electrode 20-1 for at least 5 minutes, 10 minutes, 30 minutes, 1 hour, 2 hours, 5 hours, 12 hours, 24 hours, 2 days, 10 days, 20 days, 50 days, the entire blooming season, the entire ripening season, the entire year, and any value in between.

In some embodiments, a plurality of plant electrodes may be connected directly to a DC power source and further to a plant electrode of a different plant. For example, a first plant electrode connected to a first tree in a row of trees in an orchard, may be connected to the DC power source, and to multiple other trees in the row may be connected to each other via conductive cables. In some embodiments, a plurality of first plant electrodes may all be connected to a single DC power source. In some embodiments, at least some of the first plant electrodes may each be connected to a single DC power source. In some embodiments, different groups of plants (e.g., female trees vs. male trees) may each be connected to a different power source for providing different electrical potential to each group of plants. In some embodiments, different groups of plants (e.g., female trees vs. male trees) may each be connected to a different power source for providing different electrical polarity to each group of plants.

In some embodiments, the plurality of plants may include at least 100 plants, at least 200 plants, at least 500 plants, at least 1000 plants, at least 2000 plants, at least 5000 plants, or more. In some embodiments, the distance between two neighboring plants (e.g., the trunk of a tree, or stalk of a plant) in a row of plants may be at least 0.1, meter 0.2 meter, 0.5 meter, 0.85 meter, 1 meter, 1.5 meter, 2 meters, 5 meters, 7 meters, 9 meters, 10 meters or more.

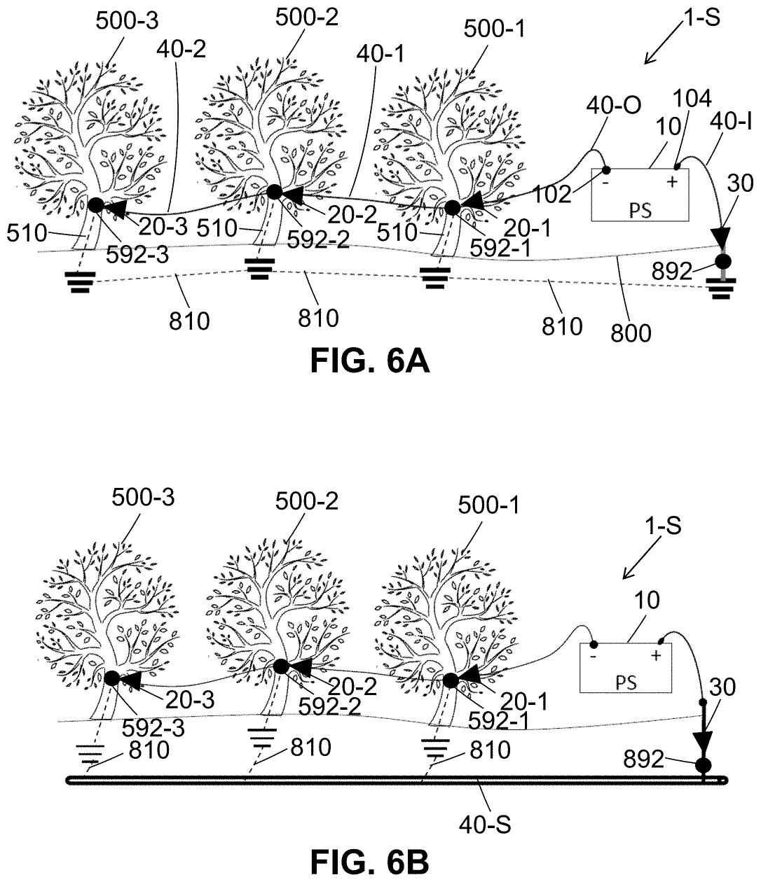

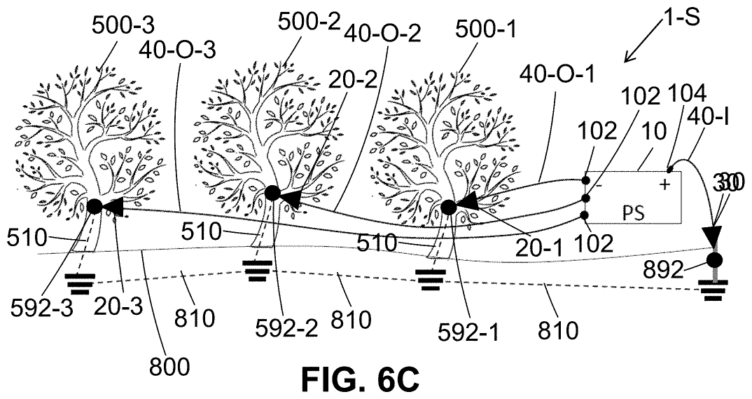

Referring now to FIGS. 6A-C schematically illustrating a side view of some embodiments of a stationary plant system for manipulating an electrical potential of a plurality of plants, comprising a power source electrically connected to a plurality of plants in parallel, according to some embodiments of the invention. It should be noted that the embodiment of electrically connecting the stationary plant system 1-S to three plants 500 in parallel is given as an example only. Any number of plants 500 that are electrically connected in parallel by the stationary plant system 1-S is under the scope of the present subject matter. In addition, the linear arrangement of the plants 500 that are electrically connected in parallel, as shown in FIGS. 6A-C is also given as an example only. Any arrangement of the plants 500 that are electrically connected in parallel, for example multiple lines of plants 500, is also under the scope of the present subject matter. According to one embodiment, the plants 500 that are electrically connected to the power source 10 in parallel are similar, for example trees of the same genus, or species; for example, the connected plants 500 are almond trees. According to another embodiment, the plants 500 that are electrically connected to the power source 10 in parallel are not similar, for example trees of different genera, or species; for example, some of the connected plants are almond tree, and others are apple trees.

FIG. 6A shows the stationary plant system 1-S electrically connecting three plants 500 in parallel. It should be noted that the embodiment of electrically connecting the stationary plant system 1-S to three plants 500 in parallel, as shown in FIG. 6A and the following FIGS. 6B-C, is given as an example only. Any number of plants 500 that are electrically connected in parallel by the stationary plant system 1-S is under the scope of the present subject matter.

FIG. 6A illustrates a power source 10 electrically connected in parallel to three plants 500 with electrically conductive cables 40, and to the growth medium 800, for example soil 800 in which the plants 500 are planted, similarly to the stationary plant system 1-S shown in FIG. 4E, thereby generating a closed electrical circuit in which an electrical current can flow from the power source 10 to the plants 500 through electrically conductive cables 40, through the plants 500 as depicted with dashed lines 510, through the growth medium 800 as depicted with dashed lines 810, and back to the power source 10 through the inward electrically conductive cable 40-I. Also in this embodiment, the stationary plant system 1 comprises three plant electrodes—20-1, 20-2 and 20-3, when each one of them is electrically and mechanically connected to a plant contact point—592-1, 592-2 and 592-3 at each plant 500. The closed electrical circuit allows manipulation of the electrical potentials of the plants 500 that are electrically connected in parallel, as described above.

In the embodiment illustrated in FIG. 6A, the power source 10 is electrically and mechanically connected to a closest first plant 500-1 with an outward electrically conductive cable 40-O, the first plant 500-1 is electrically connected to a next second plant 500-2 in the row with a first electrically conductive cable 40-1, and the second plant 500-2 is electrically connected to a third plant 500-3 in the row with a second electrically conductive cable 40-2. The electrical and mechanical connection to the plants 500 is achieved with the plant electrodes —20-1, 20-2 and 20-3, shown in FIG. 6A. In addition, the growth medium 800 is electrically connected to the power source 10 with a grounding electrode 30 and with an inward electrically conductive cable 40-I. This configuration of the stationary plant system 1-S allows formation of an electrical circuit in which the plants 500 are electrically connected in parallel, when the electrical circuit allows flow of an electrical current from the power source 10 through the plants 500 and the electrically conductive cables 40 that connect the plants 500, and then through the growth medium 800 back to the power source 10 to close the electrical circuit.

FIG. 6B illustrates another embodiment of the stationary plant system 1-S and the parallel electrical circuit shown in FIG. 6A. For the sake of simplicity, reference numbers of components that are shown in FIG. 6A have been omitted in FIG. 6B. According to the embodiment shown in FIG. 6B, the stationary system 1-S further comprises at least one in-growth medium electrically conductive cable 40-S that is configured to electrically connect to the power source 10 and conduct an electrical current Electrically conductive cable 40-S is not isolated to allow conducting electricity to the medium (e.g., soil). as part of the electrical circuit, for example a parallel electrical circuit, as shown in FIG. 6B. According to this embodiment, the in-growth medium electrically conductive cable 40-S is electrically and mechanically connected to the growth medium contact point 892 to which the grounding electrode 30 is electrically and mechanically connected. According to another embodiment, the in-growth medium electrically conductive cable 40-S is elongated and is configured to be buried in the growth medium 800, or for example placed in a tunnel in the growth medium 800, or in a ditch dug in the growth medium 800, in the vicinity of the plants 500, in a manner that allows electrical connection of the in-growth medium electrically conductive cable 40-S with the growth medium 800 that is in the vicinity of the plants 500. Dashed lines 810 in FIG. 6B designate the parts of the electrical circuit through the growth medium 800 that connect to the in-growth medium electrically conductive cable 40-S. Thus, the in-growth medium electrically conductive cable 40-S facilitates the conduction of the electrical current through the growth medium 800.

Any type of in-growth medium electrically conductive cable 40-S is under the scope of the present subject matter, for example a rod, a strip, a pipe, a water drip line and the like, given that it is electrically conductive. For example, a water pipe made of an electrically conductive material can serve as an in-growth medium electrically conductive cable 40-S. Alternatively, or additionally, an electrically conductive liquid, for example water, that is in the water pipe, can serve as the in-growth medium electrically conductive cable 40-S.

It should be noted in this regard that when the growth medium 800 in the vicinity of the plants 500 is moist, for example during to natural rainfalls, or due to irrigation, for example, but not necessarily, the moisture in the growth medium 800 can affect the electrical conductivity of the growth medium 800. The electrical conductivity of the growth medium 800 can also be affected by changing the chemical composition of the growth medium 800, for example by modifying the amount of salts per unit volume of the growth medium 800.

At least one in-growth medium electrically conductive cable 40-S can be placed in a vicinity of a row of plants 500, for example a row of trees 500. In another embodiment, one in-growth medium electrically conductive cable 40-S can be placed in a vicinity of a row of plants 500; or in-between multiple rows of plants 500, for example two rows of plants 500, thus serving the multiple rows of plants 500 in terms of conductivity of an electrical current as part of the electrical circuit formed by the stationary plant system 1-S.

FIG. 6C illustrates a power source 10 electrically connected in parallel to three plants 500 with electrically conductive cables 40, and to the growth medium 800, for example soil 800 in which the plants 500 are planted, similarly to the stationary plant system 1-S shown in FIG. 6A, thereby forming an electrical circuit that flows from the power source 10 to the plants 500 through electrically conductive cables 40, through the plants 500 as depicted with dashed lines 510, through the growth medium 800 as depicted with dashed lines 810, and back to the power source 10 through an inward electrically conductive cable 40-I. Also in this embodiment, the stationary plant system 1 comprises at least one plant electrode 20 electrically and mechanically connected to at least one corresponding plant contact point 592 at each plant 500, and at least one grounding electrode 30 electrically and mechanically connected to a corresponding growth medium contact point 892 at the growth medium 800. A closed electrical circuit allows manipulation of the electrical potentials of the plants 500 that are electrically connected in parallel.

In the embodiment illustrated in FIG. 6C, the power source 10 is electrically connected with each plant 500 with a specific electrically conductive cable 40—the power source 10 is electrically connected to the first plane 500-1 with a first outward electrically conducting element 40-O-1; the power source 10 is electrically connected to the second plant 500-2 with a second outward electrically conducting element 40-O-2; and the power source 10 is also electrically connected to the third plant 500-3 with a third outward electrically conducting element 40-O-3. In addition, the growth medium 800 is electrically connected to the power source 10 with an inward electrically conductive cable 40-I. As can be further seen in FIG. 6C, the electrical and mechanical connection with the plants 500 is achieved with corresponding plant electrodes 20 that are electrically and mechanically connected to corresponding plant contact points 592 at the plants 500; and the electrical and mechanical connection with the growth medium 800 is achieved with at least one grounding electrode 30 the is electrically and mechanically connected to a corresponding growth medium contact point 892 at that growth medium 800.

According to one embodiment, when the electrical potential of a plurality of plants 500 is manipulated, the plurality of plants 500 is electrically connected in series, for example trees in a row of the trees, trees in an orchard, trees in a grove, and the like.

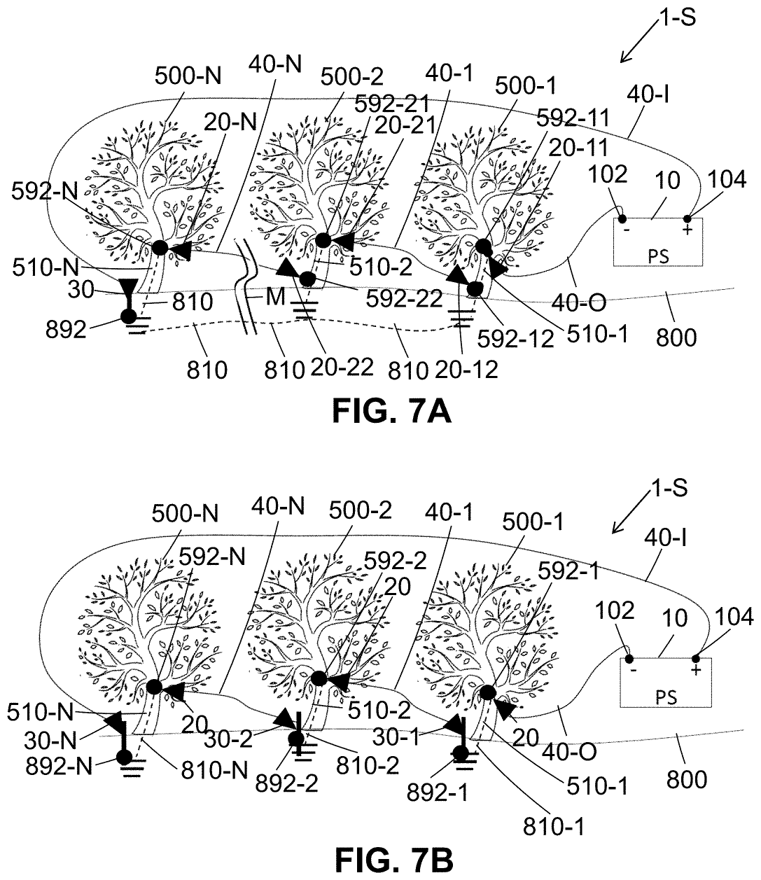

Referring now to FIGS. 7A-B schematically illustrating, according to an embodiment, a side view of some embodiments of a stationary plant system, comprising a power source electrically connected to a plurality of plants in a series. It should be noted that the embodiment of electrically connecting the stationary plant system 1-S to three plants 500 in a series is given as an example only. Any number of plants 500 that are electrically connected in series by the stationary plant system 1-S is under the scope of the present subject matter. In addition, the linear arrangement of the plants 500 that are electrically connected in a series, as shown in FIGS. 7A-B is also given an example. Any arrangement of the plants 500 that are electrically connected in a series, for example multiple lines of plants 500, is also under the scope of the present subject matter. According to one embodiment, the plants 500 that are electrically connected in a series to the power source 10 are all similar, for example trees 500 of the same genus, or species; for example, all the connected plants 500 are almond trees 500. According to another embodiment, the plants 500 that are electrically connected in a series to the power source 10 are not similar, for example trees 500 of different genera, or species; for example, some of the connected plants 500 are almond trees 500, and others are apple trees 500, or in another example—some lines of trees 500 that are electrically connected in a series are almond trees 500, and other lines of trees 500 that are electrically connected in a series are orange trees 500. In an additional example, some lines of trees 500 that are electrically connected in a series are female trees 500, and other lines of trees 500 that are electrically connected in a series are male trees 500.

FIG. 7A shows a side view of a stationary plant system, comprising a power source electrically connected to a plurality of plants in series. FIG. 7A shows the stationary plant system 1-S electrically connecting n plants 500 in a series: a first plant 500-1 that is the closest plant 500 to the power source 10, a second plant 500-2 that is the second plant 500 in the electrical circuit relative to the power source 10, and an n^{th} plant 500-N that is the last plant 500 in the electrical circuit relative to the power source 10. The notation “n” can be any number of plants 500 that are electrically connected in a series by the stationary plant system 1-S. The mark M between the second plant 500-2 and the n^{th} plant 500-N indicates that any number of plants 500, can be present in the electrical circuit between the second plant 500-2 and the n^{th} plant 500-N. In some embodiments, n can equal to 2 as well, so the second plant 500-2 is the n^{th} plant 500-N.

FIG. 7A illustrates a power source 10 electrically connected in a series to n plants 500 with electrically conductive cables 40, and to the growth medium 800, for example soil 800 in which the plants 500 are planted, thereby generating an electrical circuit that allows from of an electrical current from the power source 10 to the plants 500 through electrically conductive cables 40, through the plants 500 (either through the entire plants 500, or through parts, or portions, of the plants 500) as depicted with dashed lines 510, through the growth medium 800 as depicted with dashed lines 810, and back to the power source 10 through the inward electrically conductive cable 40-I. A closed electrical circuit allows manipulation of the electrical potentials of the plants 500 that are electrically connected in a series.

Also in the stationary plant system 1-S shown in FIGS. 7A-B, plant electrodes 20 are used to electrically and mechanically connect to corresponding plant contact points 592 is the plants 500, and a grounding electrode 30 is used to electrically and mechanically connect to a corresponding growth medium contact point 892 in the growth medium 800, as described in detail above.

According to one embodiment, shown in FIG. 7A, the grounding electrode 30 that is in the vicinity of plant 500-N is in a form of a conducting column, or rod, embedded in the growth medium 800. However, this embodiment should not be considered as limiting the scope of the present subject matter. Any type of grounding electrode 30 is under the scope of the present subject matter. In some embodiments, an edge of the inward electrically conductive cable 40-I can serve as a grounding electrode 30, for example by attaching or inserting the edge of the inward electrically conductive cable 40-I to or into the growth medium 800.

FIG. 7A shows a stationary plant system 1-S electrically and mechanically connected to a growth medium contact point 892 in the growth medium 800, and to plant contact points 592 in a first plant 500-1, a second plant 500-2 and an n^{th} plant 500-N. The designations of the various plant contact points 592 and the corresponding plant electrodes 20 that are electrically and mechanically connected to them are as follows: a first plant contact point in the first plant: 592-11, and a corresponding plant electrode 20-11; a second plant contact point in the first plant: 592-12, and a corresponding plant electrode 20-12; a first plant contact point in the second plant: 592-21, and a corresponding plant electrode 20-21; a second plant contact point in the second plant: 592-22, and a corresponding plant electrode 20-22; a plant contact point in the n^{th} plant: 592-N, and a corresponding plant electrode 20-N.

In the embodiment illustrated in FIG. 7A, the power source 10 is electrically connected to plant electrode 20-11 that is electrically and mechanically connected to a plant contact point 592-11 at a closest first plant 500-1 with an outward electrically conductive cable 40-O. A plant electrode 20-12 is electrically and mechanically connected to a plant contact point 592-12 at the first plant 500-1. The plant electrode 20-12 of the first plant 500-1 is electrically connected to a first electrically conductive cable 40-1, that is electrically connected plant electrode 20-21 that is electrically and mechanically connected to a plant contact point 592-21 at the second plant 500-2. In this way, the first plant 500-1 is electrically connected to the second plant 500-2. At the second plant 500-2 there is also a plant contact point 592-22 to which a plant electrode 20-22 is electrically and mechanically connected. The plant electrode 2 20-22 of the second plant 500-2 is also electrically connected, with an n^{th} electrically conductive cable 40-N to a plant electrode 20-N that is electrically and mechanically connected to a plant contact point 592-N at the n^{th} plant 500-N (last plant 500) in the row of plants 500. At the growth medium 800 near the n^{th} plant 500-N there is a growth medium contact point 892 to which a grounding electrode 30 is electrically and mechanically connected. The grounding electrode 30 is also electrically connected to the power source 10 with an inward electrically conductive cable 40-I. This configuration of the stationary plant system 1-S allows formation of an electrical circuit in which the plants 500 are electrically connected in series.

In the configuration of the stationary plant system 1 illustrated in FIG. 7A, an electrical circuit is formed and allows flow of an electric current in the following route: anode 102 of the power source 10—outward electrically conductive cable 40-O—plant electrode 20-11 electrically and mechanically connected to a plant contact point 592-11 at the first plant 500-1— through the trunk of the first plant 510-1—plant contact point 592-12 at the first plant 500-1— plant electrode 20-12 electrically and mechanically connected to plant contact point 592-12 at the first plant 500-1—first electrically conductive cable 40-1—plant electrode 20-21 electrically and mechanically connected to plant contact point 592-21 at the second plant 500-2—through the trunk of the second plant 510-2—plant contact point 592-22 at the second plant 500-2—plant electrode 20-22 electrically and mechanically connected to plant contact point 592-22 at the second plant 500-2—n^{th} electrically conductive cable 40-N—plant electrode 20-N electrically and mechanically connected to plant contact point 592-N at the n^{th} plant 500-N— through the trunk of the n^{th} plant 510-N—through the growth medium 810—growth medium contact point 892—grounding electrode 30 electrically and mechanically connected to the growth medium contact point 892—inward electrically conductive cable 40-I—cathode 104 of the power source 10, where the electrical circuit is closed.

FIG. 7B illustrates a stationary plant system 1-S and an electrical circuit as shown in FIG. 7A, except that a grounding electrode 30 is electrically and mechanically connected to a plant contact point 892 positioned near each plant 500, and one plant electrode 20 is electrically and mechanically connected to a plant contact point 592 at each plant 500. In addition, the grounding electrode 30 electrically and mechanically connected to the plant contact point 892 near the last plant 500, which is the third plant 500-3, is electrically connected with an inward conductive element 40-I to the cathode 104 of the power source 10. This configuration of the stationary plant system 1-S also forms an electrical circuit in which the plants 500 are electrically connected in a series, when the electrical circuit flows, for example, in the following route: anode 102 of power source 10—outward electrically conducting element 40-O—plant electrode 20 electrically and mechanically connected to a first plant contact point 592-1 at first plant 500-1—through first plant 510-1—through growth medium 810-1 near first plant 500-1—grounding electrode No. 1 30-1 electrically and mechanically connected to a first growth medium contact point 892-1 in the growth medium 800 near first plant 500-1—first electrically conductive cable 40-1—plant electrode 20 electrically and mechanically connected to a second plant contact point 592-2 at second plant 500-2—through second plant 510-2— through growth medium 810-2 near second plant 510-2—grounding electrode No. 2 30-2 electrically and mechanically connected to a second growth medium contact point 892-2 in the growth medium 800 near second plant 500-2—second electrically conductive cable 40-2—plant electrode 20 electrically and mechanically connected to an n^{th} plant contact point 592-N at an n^{th} plant 500-N—through n^{th} plant 500-N—through growth medium 810-N near n^{th} plant 500-N—grounding electrode No. n 30-N electrically and mechanically connected to an n^{th} growth medium contact point 892-B in the growth medium 800 near n^{th} plant 500-N—inward electrically conductive cable 40-I—cathode 104 of power source 10.

It should be noted again that the configuration of electrical connection of the anode 102 and cathode 104 to the other parts of the stationary plant system 1-S that is shown in FIG. 7A-B in particular, and all the relevant drawings in general, is given as an example only, and that an opposite electrical connection of the anode 102 and cathode 104 is also under the scope of the present subject matter. According to yet another embodiment, the plurality of plants 500 is electrically connected in a combination of series connection and parallel connection.

Referring now to FIGS. 8A-8C and 8D-8E schematically illustrating, according to some embodiments, a top view and a side view of a plant electrode 20. In some embodiments, plant electrode 20 of FIGS. 8A-8C and 8D-8E is to be used with the method of FIG. 5B. According to one embodiment, the plant electrode 20 is configured to electrically connect to an electrically conductive cable 40, and to electrically and mechanically connect to a plant 500, and conduct an electrical current from the electrically conducting element 40 to the plant 500. Some examples of the plant electrode 20 are described hereinafter.

It should be noted that in some occasions, electrical and mechanical connection of the plant electrode 20 to a surface of a plant 500 can ionize air particles in the vicinity of the plant electrode 20. Thus, airborne particles, solid or fluid, have a capacity to carry an electrical charge. An electrical charged particle has an electrical potential, that can be positive, or negative, or neutral (namely, without excess electrical charge). On the other hand, electrically charged airborne pollen grains, drifting in the air, can be affected by the electrically charged airborne particles. If the electrical charge of the pollen grains and of the airborne particles is in the same polarity—for example, both are positively charged, or negative charged, they can be repelled from the vicinity of the plant electrode 20, and as a result, from the plant 500. However, If the electrical charges of the pollen grains and the airborne particles are of opposite polarities—for example, the airborne particles are negatively charged, and the pollen grains are positively charged, there is a possibility that the electrical charge of the pollen grains will be neutralized, or reduced.

The plant electrode 20, shown in FIGS. 8A-8E, is configured to electrically connect to an electrically conductive cable 40, and electrically and mechanically connect to a surface of the plant 500, and conduct electrical current to the surface of the plant 500. According to one embodiment, the plant electrode 20 is configured to attach to any surface of a plant 500, thereby affecting the electrical potential of the plant 500. In this regard, it should be mentioned that an outer surface of the plant 500 acts as a resistor in an electrical circuit, namely resists conduction of an electrical current therethrough. The thicker the outer layer of the plant 500—the more resistant it is to the conduction of the electrical current. For example, a bark of a trunk, or branches, of a tree 500 are highly electrically resistant, and can be considered as electrical insulators, for example within voltage ranges of substantially 0 to 500 Volts. Therefore, when the plant electrode 20 electrically and mechanically connects to a plant contact point 592 on a surface of a trunk of a tree 500, the electrical potential of the plant 500 cannot be manipulated. On the other hand, an epidermal layer of a leafy part of a plant 500, for example a leaf of a plant 500, or a stem of a herbal plant 500, is less electrically resistant. Therefore, when the plant electrode 20 electrically and mechanically connects to a plant contact point 592 on the surface of a part of a herbal plant 500, the electrical potential of the herbal plant 500 can be manipulated, even though there can be some loss of potential. When the plant electrode 20 electrically and mechanically connects to a plant contact point 592 on a surface of a trunk of a tree 500, the electrical potential of air, or environment, surrounding the plant 500 can be manipulated.

According to one embodiment, the plant electrode 20 comprises a surface body 632 made at least partially of an electrically conductive material. FIG. 8A shows a plant electrode 20 comprising a surface body 632 in a form of a band, and alternatively, a band closure element 631, made of at least partially electricity conducting material. The surface body 632 in a form of a band is configured to embrace a stem of a herb, or bush; or a branch or a trunk of a tree, for example, as shown hereinafter. In another embodiment, the surface body 632 is electrically non-conductive, and the plant electrode 20 further comprises a surface conductor 634 that is attached to the surface body 632, electrically connected to an electrically conducting element 40, and configured to electrically and mechanically connect to the surface of the plant 500. Thus, the surface conductor 634 is configured to receive an electrical current from the electrically conductive cable 40 and conduct the electrical current to the surface of the plant 500.

According to another embodiment, shown in FIGS. 8B and 8C, the plant electrode 20, as shown in FIG. 8A, further comprises at least one affixing element 637 that is attached to the surface body 632 and configured to affix to the surface of the plant 500, for example to a bark of a tree, and prevent undesired detachment of the plant electrode 20 from the surface of the plant 500. According to yet another embodiment, the at least one affixing element 637 is made of an electrically conductive material. This embodiment is important for providing conduction of an electrical current into inner tissues, or layers, of the plant 500.

According to one embodiment, the affixing element 637 has a tack-like shape, or a nail-like shape, or a blade-like shape, having a sharp tip, or edge, that is configured to facilitate affixing of the affixing element 637 to the surface of the plant 500, thereby preventing undesired detachment of the plant electrode 20 from the surface of the plant 500. It should be noted that the shape of the affixing element 637 illustrated in FIG. 8B is given as an example only, and should not be considered as limiting the scope of the present subject matter. The affixing element 637 can have any shape the allows affixing of the affixing element 637 to the surface of the plant 500. In some embodiments two or more affixing elements 637 may be connected evenly around body 632. The angular distance between the electrodes may be calculated using the following equation:

Angular distance=360/n±20°,

Where n is the number of electrodes.

For example, two affixing elements 637 may be connected at two opposite sides of body 632, for example, at 180±20° from each other. In yet another example, three affixing elements 637 may be connected at 120±20° from each other.

FIGS. 8C-8D show a plant 500, for example a tree 500, and a plant electrode 20 electrically and mechanically connected to a plant contact point 592 at a part of the plant 500, with an electrically conductive cable 40 electrically connected to the surface body 632 of the plant electrode 20. The plant electrode 20 is configured to electrically and mechanically connect to any part of the plant 500. For example, in an embodiment of the plant 500 as a tree 500, the plant electrode 20 is configured to electrically and mechanically connect to a trunk 550 of the tree 500 given as an example. The plant electrode 20 can electrically and mechanically connect, for example, to at least one branch 555 of a tree 500, or to at least one leaf 557; or in embodiments of the plant 500 as a herb, the plant electrode 20 can electrically and mechanically connect to at least one stem of at least one herb, at least one leaf of the at least one herb, and the like.

FIG. 8C shows an embodiment of the plant electrode 20 that comprises a surface body 632 in a form of a band made of at least partially electricity conducting material. On the other hand, FIG. 8D shows an embodiment of the plant electrode 20 that comprises a surface body 632 in a form of a coil made of at least partially electricity conducting material. The surface body 632 in the form of a coil is configured to embrace a part of the plant 500, for example a trunk 550 of a tree 500, and conduct an electrical current to the surface of the part of the plant 500.

As can be seen in FIG. 8D, an electrically conductive cable 40 is electrically connected to the surface body 632 having a coil-like structure. Thus, according to one embodiment, the surface body 632 having a coil-like structure is electrically connected to an electrically conductive cable 40. This electrically conductive cable 40 can be an outward electrically conductive cable 40-O electrically connected to the power source 10; or an electrically conductive cable 40 that is electrically connected to another plant 500, for example in the embodiment described in FIGS. 6A-C and 7A-B of multiple plants 500 electrically connected in a serial, or parallel, electrical circuit; or any other type of electrically conducting element 40 according to embodiments of the present subject matter.

As further shown in FIG. 8D, an additional electrically conductive cable 40 is electrically connected to the surface body 632 having a coil-like structure. Thus, according to a further embodiment, the surface body 632 having a coil-like structure is electrically connected to an additional electrically conductive cable 40. This electrically conductive cable 40 can be an inward electrically conductive cable 40-I electrically connected to the power source 10; or an electrically conductive cable 40 that is electrically connected to another plant 500, for example in the embodiment described in FIGS. 6A-6C and 7A-7B of multiple plants 500 electrically connected in a serial, or parallel, electrical circuit; or any other type of electrically conducting element 40 according to embodiments of the present subject matter.

It should be noted that the structure of the plant electrode 20 that is shown in FIGS. 8A-8D is given as an example only. For example, the plant electrode 20 can have a surface body 632 having a substantially flat structure, for example a sheet-like structure, that is configured to attach, or adhere, to a surface of a part of a plant 500, and conduct electricity to the surface of the plant 500. In another specific example, the surface body 632 can adhere to a surface of a part of a plant 500 by using glue, preferably electrically conductive glue, that can improve the electrical conductivity between the surface body 632 and the surface of the part of the plant 500. In another specific example, the surface body 632 Includes a conductive element electrically connected to at least two affixing elements 637. In yet another specific example, the at least two affixing elements 637 can be separated from the surface body 632. In still another specific example, the at least two affixing elements 637 can be mechanically and electrically connected to the plant 500 prior to being electrically connected to the surface body 632.

Referring now to FIG. 9A schematically illustrating, according to some embodiments, a side view of a plant electrode configured to electrically and mechanically connect to at least one inner tissue, or layer of a plant. The plant electrode 20, shown in FIG. 9A, is configured to electrically connect to the power source 10, electrically and mechanically connect to a plant contact point 592 in at least one inner tissue, or layer of the plant 500, and to conduct an electrical current to the at least one inner tissue, or layer of the plant 500.

According to one embodiment, the plant electrode 20 is at least partially electrically conductive, namely made at least partially of an electricity conductive material. FIG. 9A shows an plant electrode 20. The structure of the plant electrode 20 facilitates penetration of the electrode 20 into inner tissues, or layers of a plant 500 and conduct an electrical current to the inner tissues, or layers of the plant 500. A structure of the plant electrode 20, shown in FIG. 9A, is a substantially elongated sharp structure. Thus, according to this embodiment, the plant electrode 20 comprises at least one affixing element 652. In the embodiment shown in FIG. 9A, the plant electrode 20 comprises one affixing element 652. Hereinafter, other embodiments of the plant electrode 20 that comprise multiple affixing elements 652, are shown. The affixing element 652 is configured to penetrate into the plant 500. In another embodiment, the structure of the affixing element 652 is elongated, thin, and the like. In yet another embodiment, the structure of the affixing element 652 is a blade-like structure with a sharp uniform, or serrated, edge.

According to another embodiment, the plant electrode 20 optionally comprises a sharp tip 654 at one edge of the affixing element 652. The tip 654 is configured to puncture a surface of a part of a plant 500 and facilitate penetration of the affixing element 652 into inner tissues, or layers of the plant 500.

The dashed line 651 inside the affixing element 652 of the plant electrode 20 designates passage of electrical current from the electrically conductive cable 40 through the affixing element 652, and optionally also through the tip 654, in a manner that allows conduction of the electrical current from the plant electrode 20 to inner tissues, or layers of the plant 500.

Referring now to FIG. 9B schematically illustrating, according to an embodiment, a side view of a plant electrode configured to electrically and mechanically connect to at least one inner tissue, or layer of a plant. FIG. 9B shows a plant 500, for example a tree 500, and a plant electrode 20 electrically and mechanically connect to a part of the plant 500, with an electrically conductive cable 40 electrically connected to the plant electrode 20. The plant electrode 20 is configured to electrically and mechanically connect to any part of the plant 500. For example, in an embodiment of the plant 500 as a tree 500, the plant electrode 20 is configured to electrically and mechanically connect to a trunk 550 of the tree 500, as shown in FIG. 9B. However, this embodiment is given as an example only. The plant electrode 20 can electrically and mechanically connect, for example, to at least one branch 555 of a tree 500; or to at least one leaf 557 of the plant; or in embodiments of the plant 500 as a herb, the plant electrode 20 can electrically and mechanically connect to at least one stem of at least one herb, at least one leaf of at least one herb, and the like.

As can be seen in FIG. 9B, at least part of the affixing element 652 of the plant electrode 20 penetrates into inner tissues of the plant 500, in this embodiment, into inner tissues of a trunk 550. In one embodiment, in order to penetrate the affixing element 652 into the trunk 550, the tip 654 of the plant electrode 20 can be used to puncture the surface of the trunk 550 in order to facilitate penetration of the affixing element 652 into the inner tissues of the trunk 550. In another embodiment, as designated in FIG. 9B with dashed line 651, electrical current is conducted through an interior, and in some cases on a surface of the affixing element 652 of the plant electrode 20 into inner tissues of the trunk 550 of the tree 500.

Refereeing now to FIGS. 9C and 9D schematically illustrating, according to an embodiment, a side view of affixing elements of a plant electrode configured to electrically and mechanically connect to at least one inner tissue, or layer of a plant. Affixing element 652-1 is inserted into a heartwood 525, affixing element 653-2 is inserted into bark 515 and affixing element 653-2, acting as the grounding electrode is inserted into a sapwood 520. In some embodiments, affixing element 653-1 is connected to a first pole of power source 10 (which can be either DC power source or AC power source) and affixing element 653-2 is connected to the second pole of power source 10, thereby closing the electrical circuit between the different layers of plant 500. In FIG. 9C affixing element 653-3 is connected to a conducting element. In FIG. 9D affixing element 653-3 is connected to a resistor, thereby forming coaxial transmission line. The resistor allows better electrical energy coupling between power source 10 and plant 500. Since the resistance of the transmission line depends on the diameter of the conductors, it is possible to control the point to which the majority of energy will be provided by choosing to connect at least one of the affixing elements to a thinner branch, thus directing the current to flow to this section of the tree.

It should be noted that the structure of the plant electrode 20 that is shown in FIGS. 9A-9D is given as an example only. For example, the plant electrode 20 can have a substantially elongated structure that is optionally sharp at one side, for example a nail-like structure, a screw-like structure, a peg-like structure, and the like, that is configured to penetrate into inner tissues of a plant 500, and conduct electricity to the inner tissues of the plant 500. Other embodiments of the structure of the plant electrode 20 are illustrated hereinafter.

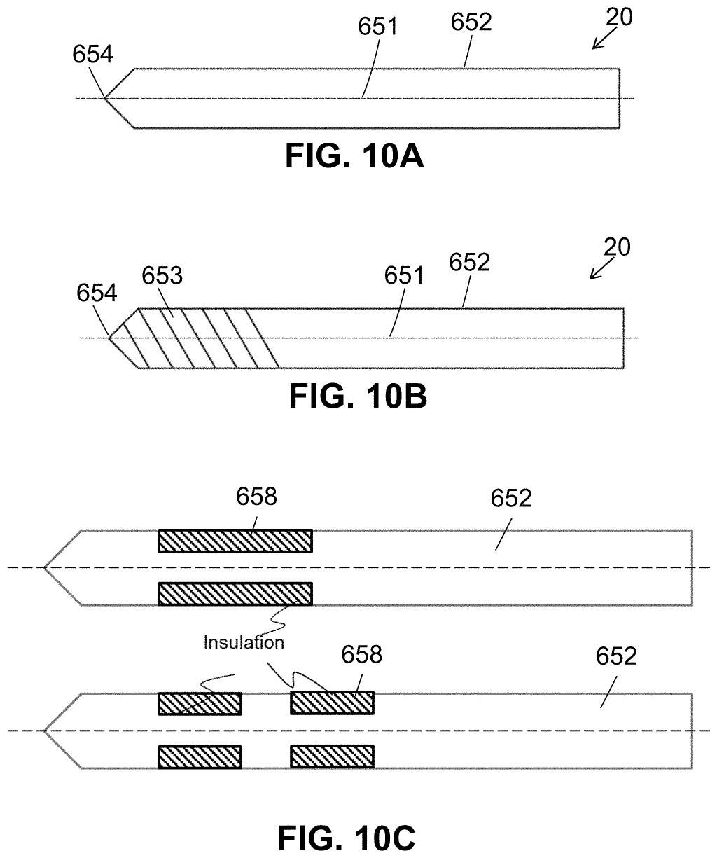

Referring now to FIGS. 10A-B, schematically illustrating, according to an embodiment, top longitudinal section views of various embodiments of a plant electrode. As mentioned above, according to the various embodiments shown in FIGS. 10A-B, the plant electrode 20 comprises a affixing element 652 that has a structure that facilitates penetration of the affixing element 652 into inner tissues of a plant 500, and conduct electrical current to the inner tissues, namely an elongated structure, and an optional sharp edge 654.

According to one embodiment, illustrated in FIG. 10A, the affixing element 652 of the plant electrode 20 is entirely electrically conductive. In other words, this embodiment of the plant electrode 20 is configured to conduct an electrical current throughout the entire surface of the affixing element 652, thereby allowing conduction of an electrical current to multiple tissues of the plant 500 that are in contact with the surface of the affixing element 652 of the plant electrode 20.

According to one embodiment, illustrated in FIG. 10B, the plant electrode 20 further comprises screw threads 653 on a surface of the affixing element 652, for example adjacent to the tip 654. This embodiment confers to the affixing element 652 a screw-like structure, facilitating penetration of the affixing element 652 into inner tissues of the plant 500. For example, the affixing element 652 comprising screw threads 653 is configured to be screwed into a part of a plant 500, for example into a trunk 550 of a tree 500, a branch 555 of a tree 500, and the like. It should be noted that the embodiment of the screw threads 653 is not restricted to a plant electrode 20 that is entirely electrically conductive, as shown in FIG. 6A. Also, other embodiments of the plant electrode 20, that are shown hereinafter, can comprise screw threads 653.

According to one embodiment, illustrated in FIG. 10C, the affixing element 652 of the plant electrode 20 at least partially comprising one or more isolation elements 658 configured to isolate at a portion of the length of affixing element 652 from electrically be connected to plant 500. In this embodiment, only portions of affixing element 652 not covered by isolation elements 658 can deliver electrical current to the plant, thereby ensuring that specific layers of the plant (e.g., bark 515, sapwood 520, heartwood 525 etc., illustrated in FIGS. 9C and 9D) are being included in the electrical circuit. Therefore, the current may be forced to flow via specific layers of the trunk.

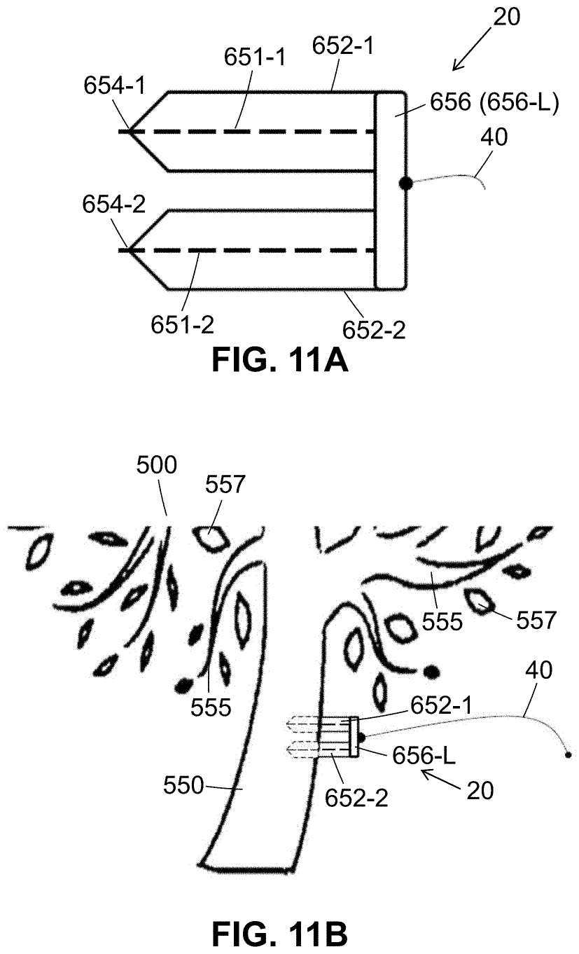

Referring now to FIG. 11A, schematically illustrating, according to an embodiment, a side view of a plant electrode 20 comprising a plurality of affixing elements connected to a linear connector. According to one embodiment, the plant electrode 20 comprises a plurality of affixing elements 652, electrically connected to a connector 656 configured to connect to the plurality of affixing elements 652, and to an electrically conductive cable 40, in a manner that allows conduction of an electrical current from the electrically conductive cable 40 to the plurality of affixing elements 652. The connector 656 has any shape that allows the connector 656 to connect to the plurality of affixing elements 652, and to an electrically conductive cable 40. According to one embodiment, a surface of the connector 656 that can be in contact with a surface of the plant 500 is covered with an electrically insulating layer in order to prevent conduction of an electrical current to the surface of the plant 500, thus allowing conduction of an electrical current only into inner tissues of the plant 500 through the at least one affixing element 652. This embodiment of the electrically insulating layer is relevant to most of the different shape embodiments of the connector 656 mentioned hereinafter. In the embodiment illustrated in FIG. 11A, the connector 656 is linear, designated hereinafter “linear connector 656-L”. Furthermore, in the embodiment illustrated in FIG. 11A, the plurality of affixing elements 652 is two affixing elements 652—a first affixing element 652-1 and a second affixing element 652-2. Accordingly, the plant electrode 20 comprises a first tip 654-1 at the first affixing element 652-1, and a second tip 654-2 at the second affixing element 652-2. In addition, the first affixing element 652-1 allows conduction of a first electrical current, depicted with dashed line 651-1, to inner tissues of the plant 500, and the second affixing element 652-2 allows conduction of a second electrical current, depicted with dashed line 651-2, to inner tissues of the plant 500.

It should be noted that the embodiment illustrated in FIG. 11A is given as an example only, and should not be considered as limiting the scope of the present subject matter. The plant electrode 20 can comprise any number of affixing elements 652, for example, a plurality of affixing elements 652 that are different in length, or in shape, or in length and shape.

According to one embodiment, the plurality of affixing elements 652 is parallel one to the other, as can be seen in FIG. 11A. However, it should be additionally noted that this embodiment as illustrated in FIG. 11A should not be considered as limiting the scope of the present subject matter. Any orientation of the plurality of affixing elements 652, one relative to the other, is under the scope of the present subject matter.

Referring now to FIG. 11B, schematically illustrating, according to an embodiment, a side view of a plant electrode comprising a plurality of affixing elements connected to a linear connector, electrically and mechanically connected to a plant. As can be seen in FIG. 11B, the plurality of affixing elements 652 of the plant electrode 20 is configured to penetrate into a part of a plant 500, and conduct electrical current to inner tissues of the plant 500, similarly to the aforementioned embodiments of the plant electrode 20, according to which the plant electrode 20 comprises one affixing element 652. In addition, all the aforementioned embodiments of the plant electrode 20 that comprises one affixing element 652, are relevant also to the plant electrode 20 that comprises a plurality of affixing elements 652.

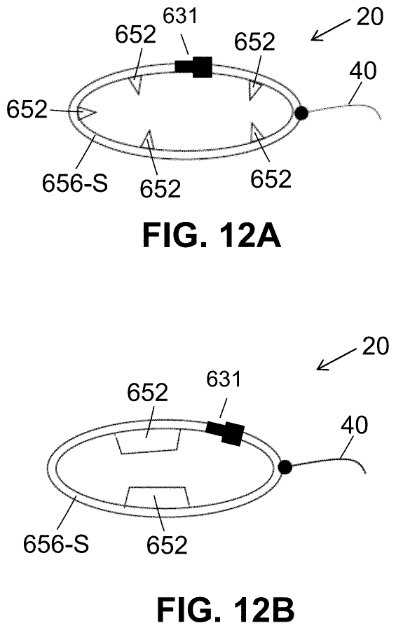

Referring now to FIGS. 12A-B, schematically illustrating, according to some embodiments, a top view of a plant electrode comprising at least one affixing element connected to a surface attaching connector. According to one embodiment, the plant electrode 20 comprises at least one affixing element 652 connected to another embodiment of the connector 656—a surface attaching connector 656-S, and alternatively a band closure element 631. The surface attaching connector 656-S is configured to attach to a surface of a part of a plant 500, thereby facilitating penetration of the at least one affixing element 652 into inner tissues of the plant 500. For example, the surface attaching connector 656-S has a shape of a band that is configured to embrace a part of plant 500, for example a stem of a herb, or bush; or a trunk of a tree, and the like. According to another embodiment, the band closure element 631 is configured to tighten the surface attaching connector 656-S having a shape of a band and insert at least one affixing element 652 into inner tissues of the plant 500. It should be noted, though, that the embodiment of the surface attaching connector 656-S that has a band-like shape is given as an example only and should not be considered as limiting the scope of the present subject matter. Any shape of the surface attaching element 656-S that allows attachment of the surface attaching element 656-S to a surface of a part of a plant 500, and thereby penetration of the at least one affixing element 652 into inner tissues of the plant 500, is under the scope of the present subject matter.

According to one embodiment, the surface attaching connector 656-S is additionally configured to conduct an electrical current from the electrically conductive cable 40, that is electrically connected to the surface attaching connector 656-S, to the at least one affixing element 652. Thus, according to another embodiment, the surface attaching connector 656-S is electrically conductive. However, in some embodiments there can be a desire to prevent conduction of the electrical current to the surface of the plant 500 to which the surface attaching connector 656-S is attached. This can be achieved, for example, by covering the surface attaching element 656-S with an electrically insulating coating. Alternatively, the plant electrode 20 can comprise at least one electrical wire, for example, that electrically connects the electrically conducting element 40 to the at least one affixing element 652.

FIGS. 12A-B further illustrate two embodiments of the shape of the at least one affixing element 652. According to the embodiment illustrated in FIG. 12A, the at least one affixing element 652 has a tack-like shape, or a nail-like shape, having a sharp tip that is configured to facilitate penetration of the at least one affixing element 652 into inner tissues of the plant 500. According to another embodiment, illustrated in FIG. 12B, the at least one affixing element 652 has a blade-like shape, having a sharp edge that is configured to facilitate penetration of the at least one affixing element 652 into inner tissues of the plant 500. It should be noted that the shapes of the affixing element 652 of the plant electrode 20 illustrated in FIGS. 12A-B, and herein in general, are given as an example only and should not be considered as limiting the scope of the present subject matter. Any shape of the affixing element 652 that facilitates penetration of the affixing element 652 into a part of a plant 500 and conduct an electrical current to inner tissues of the plant 500 is under the scope of the present subject matter.

According to one embodiment, the stationary plant system 1-S for manipulating the electrical potential of at least one plant is configured to control the electrical potential of the at least plant 500, or of at least one part of the at least one plant 500. According to another embodiment, the controlling of the electrical potential is increasing the electrical potential, or decreasing the electrical potential. According to yet another embodiment, the controlling of the electrical potential is switching a polarity of the electrical potential. According to still another embodiment, the controlling of the electrical potential is a combination of increasing, or decreasing, the electrical potential, and switching the polarity of the electrical potential.

According to one embodiment, the stationary plant system 1-S is configured to control a potential difference between at least one stigma of at least one flower of at least one plant 500, and the growth medium 800, for example soil 800 serving as electrical ground. This embodiment is achieved by electrically and mechanically connecting a power source 10 to the at least one plant 500 and to the growth medium 800 that is in contact with the at least one plant 500, for example as illustrated in FIGS. 4A and 5, thereby generating an electrical circuit between the power source 10, the at least one plant 500, and the growth medium 800 that is in contact with the at least one plant 500, wherein the growth medium 800 serves as an electrical ground in the electrical circuit.

According to one embodiment, the stationary plant system 1-S comprises a plurality of plant electrodes 20 configured to electrically and mechanically connect to separate plants 500. According to another embodiment, a distance between two adjacent plants 500 is at least one 1 meter.

According to one embodiment, the stationary plant system 1-S comprises at least one grounding electrode 30, when the number of grounding electrodes 30 is lower than a number of the plant electrodes 20. For example, the stationary plant system 1-S comprises two plant electrodes 20, each plant electrode 20 is electrically and mechanically connected to a separate plant 500; and one grounding electrode 30. In another example, the stationary plant system 1-S comprises four plant electrodes 20, each plant electrode 20 is electrically and mechanically connected to a separate plant 500; and less than four grounding electrodes 30, namely one, or two, or three grounding electrodes 30.

According to one embodiment, the plant electrode 20 is configured to electrically and mechanically connect to a plant contact point 592, and the grounding electrode 30 is configured to electrically and mechanically connect to a growth medium contact point 892, wherein a distance between the plant contact point 592 and the growth medium contact point 892 is at least substantially 10 meters.

According to one embodiment, not all plants 500, for example trees 500, in an orchard, or plantation, or row, are electrically and mechanically connected to the stationary plant system 1-S, when the stationary plant system 1-S is used for manipulating the electrical potential of a portion of the plants 500 that are electrically and mechanically connected to the stationary plant system 1-S.

According to one embodiment, the plant electrode 20 is configured to electrically and mechanically connect to a plant 500 in a form of a tree 550. More particularly, the plant electrode 20 is configured to electrically and mechanically connect to a plant contact point 592 positioned in a trunk 550 of the tree 500. A trunk 550 of a tree 500, or a stem of a plant 500, The trunk 550 connects the leafy crown with the roots. In a tree 500, the trunk 550 is the main stem apart from limbs and roots. A length of the trunk 550 is measure from the growth medium 800, or soil 800, in which the tree 550 is planted to the first branches of the crown. For example, the length of the trunk 550 is measured from the soil 800 to the first division of the trunk 550 into branches. In this regard, a crown of a tree 500 is defined as the upper part of the tree 500, composed of leaves, twigs, branches, flowers and fruits.

Plants 500, and specifically trees 500, are considered as very poor conductors of electrical current. In some cases, plants can conduct electrical current. It should be mentioned at this stage that electrical conductivity is the reciprocal of electrical resistance. Thus, different parts and different layers, or tissues, of the tree, exhibit substantially different electrical conductivity and resistance properties. For example, on one hand, the electrical resistance of an outer layer of a tree 500, for example a surface of the trunk 550 the tree 500, is very high. As a result, the outer layer of the trunk 550 is a poor electrical conductor. On the hand, the electrical resistance of inner layers, or tissues, of the trunk 550 is significantly lower that the electrical resistance of the outer layer of the trunk 550. As a result, the electrical conductivity of the internal layers, or tissues, of the trunk 550 is significantly higher than the electrical conductivity of the outer layer of the trunk 550.

In an experiment conducted by the inventor with the stationary plant system 1-S an unexpected result has been obtained. It was found that the location of the plant contact point 592, to which the plant electrode 20 is electrically and mechanically connected, has a significant effect on the electrical potential that is measured in the crown of the tree 500. The distance of the plant contact point 592 from the soil 800 in which the tree 500 is planted has a significant effect on the electrical potential of the crown, as measured with reference to the electrical ground, which is the soil 800 in which the tree 500 is planted.

In an experiment, a plant electrode 20 of the stationary plant system 1-S was electrically connected to two different positions over the height of a trunk 550 of a tree 500, and a grounding electrode 30 was electrically and mechanically connected to the soil 800 in which the tree 500 is planted, in the vicinity of the tree 500. Then, an output of the power source 10 was set to 76 Volts/5 Amp, and the electrical potential on a leaf in the crown of the tree 500 was measured. When the plant electrode 20 was electrically and mechanically connected to a plant contact point 592 in the trunk 550 at a height of substantially 1 meter above the soil 800, the measured electrical potential on the leaf was 43 Volts. However, when the plant electrode 20 was electrically and mechanically connected to a plant contact point 592 in the trunk 592 in a height of substantially 0.1 meter above the soil 800, the electrical potential on the leaf was reduced to 10 Volts. This result indicates that the higher the plant contact point 592 on the trunk, the higher is the electrical potential of the crown. This result can be attributed to the poor electrical conductivity of parts of the tree 500.

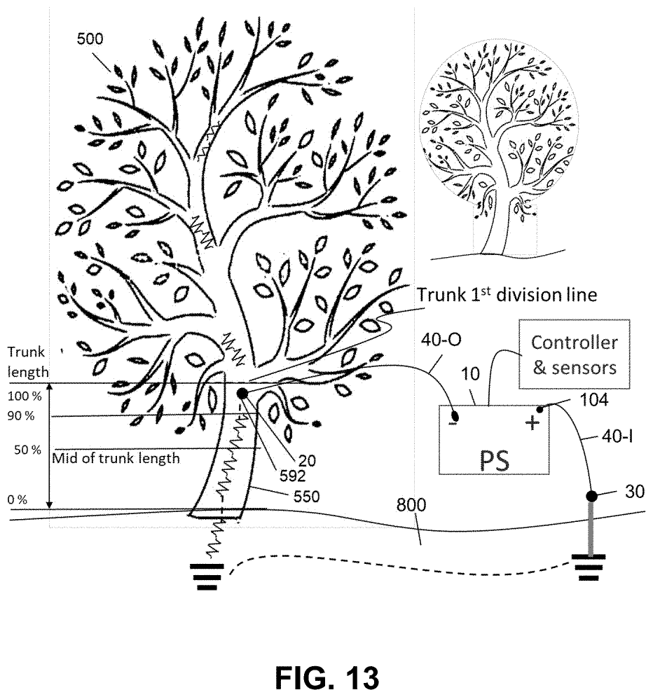

Referring now to FIG. 13, schematically illustrating, according to an embodiment, some definitions relating to a position of a plant contact point along a trunk of a tree.

“Full trunk length”, or “100% trunk length”, is defined as 100% of the trunk's length from the soil to the trunk first trunk division line, which is at the branching point of the lowest branch of the tree, measured from the surface of growth medium (e.g., soil) 800.

“Mid of trunk length”, or “50% trunk length”, is defined as a point on the trunk that is 50% of the full trunk length, as measured from the soil.

“90% trunk length” is defined as a point on the trunk that is 90% of the full trunk length, as measured from the soil. (Similarly, any length of the trunk is defined as percentage of the full trunk length from the soil).

According to one embodiment, the plant electrode 20 is electrically and mechanically connected to a plant contact point 592 at an edge a trunk 550, above substantially the 90% trunk length. According to another embodiment, the plant electrode 20 is electrically and mechanically connected above substantially the 75% trunk length.

According to one embodiment, electrically connecting the plant electrode 20 to a plant contact point 592 below the 100% trunk length, namely below the first trunk division line, results in substantially equal electrical potential in the branches of the tree that are above the first trunk division line. According to one embodiment, electrically and mechanically connecting the plant electrode 20 to a plant contact point 592 below the 100% trunk length, namely below the first trunk division line, results in less than 25% difference in electrical potential in the branches of the tree 500 that are above the first trunk division line.

According to another embodiment, electrically connecting the plant electrode 20 to a plant contact point 592 above the 100% trunk length, namely above the first trunk division line, results in non-equal electrical potential in the plurality of branches that are above the plant contact point 592. According to one embodiment, electrically and mechanically connecting the plant electrode 20 to a plant contact point 592 above the 100% trunk length, namely above the first trunk division line, results in more than 100% difference in electrical potential in the branches of the tree 500 that are above the first trunk division line.

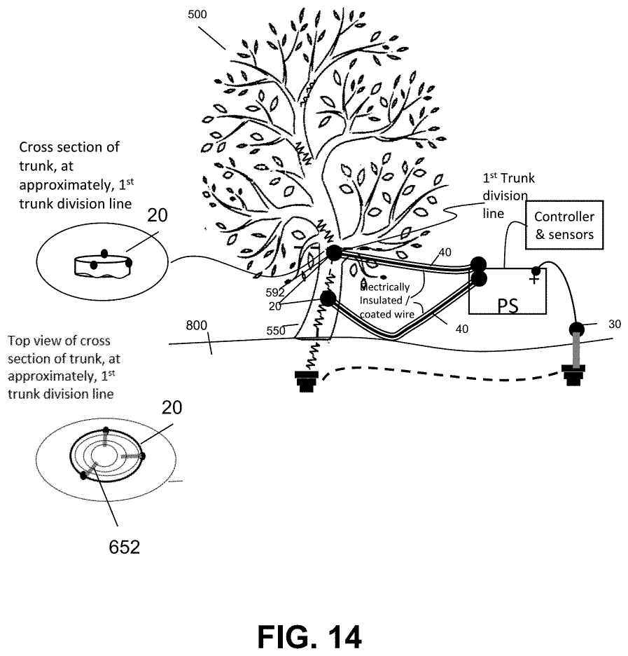

Referring now to FIG. 14, schematically illustrating embodiments of a position of plant electrodes on a trunk of a tree. According to one embodiment, the plurality of plant electrodes 20 is configured to electrically and mechanically connect to one plant 500, for example to a trunk 550 of a tree 500, at substantially the same height of the trunk 550, for example within 10% of a height of the trunk 550, around a circumference of the trunk 550. For example, two plant electrodes 20, when each plant electrode 20 is configured to electrically and mechanically connect to a separate plant contact point 592 in a trunk 550, when the two plant contact points 592 are positioned opposite one to the other, for example at an angle of substantially 180°±20% in between them. In another example, three plant electrodes 20, when each plant electrode 20 is configured to electrically and mechanically connect to a separate plant contact point 592 in a trunk, when the three plant contact points 592 are positioned at an angle of substantially 120°±20% in between them.

As shown in FIG. 14, affixing elements 652 (or 672) may penetrate into inner layers of the trunk.

FIG. 14 also shows that the plant electrodes 20 can be placed around the circumference, as well as along the trunk's length, at different heights. FIG. 14 further shows an electrically insulated electrically conductive cable 40. This electrically insulated electrically conductive cable 40, while touching the soil, does not short or drain the electrical circuit.

Referring now to FIG. 15, schematically illustrating, according to an embodiment, a stationary plant system comprising a plurality of power sources. According to one embodiment, the stationary plant system 1-S comprises a plurality of power sources 10. According to another embodiment, each power source 10 is configured to electrically and mechanically connect to a different type of plant 500, or to different rows of plants 500. For example, each power source 10 is electrically and mechanically connected to a different row of trees 500; or one power source 10 is electrically and mechanically connected to male trees 500 and another power source 10 is electrically and mechanically connected to female trees 500; or one power source is electrically and mechanically connected to trees 500 at an edge of an orchard, and another power source 10 is electrically and mechanically connected to trees 500 in a center region of the orchard. In accordance with these embodiments, each power source 10 may provide a different type of electrical current, for example in order to give rise to a different electric potential to the different types of plants 500. FIG. 15 shows: A) a stationary plant system 1-S that includes electrically insulated electrically conductive cable 40 as well as non-insulated electrically conductive cable 40; B) a non-conductive element supporting the electrically conductive cable 40 and preventing it from touching the growth medium 40; C) a distance D1 from the power source 10 to a first tree 500, a distance D2 from the power source 10 to a second tree, and distance D3 between trees 500-1 and 500-2. In some embodiments, the trees 500-1 and 500-2 are not planted in a linear fashion; D) The power source 10 is connected to, and controlled by a controller, that is further connected to sensors. The connection to the sensors can be wired or wireless. Sensors include sensors that sense for example temperature, humidity, wind condition, soil conditions, and plant parameters. In a nonlimiting example, the distance D3 between plants or tress 500 may be at least 0.5 meter, at least 0.8 meter at least 1 meter or more. Therefore, the distance D1 of the electrical contact point of the first plant 500-1 from DC power source 10 (e.g., grounding electrode 30) may be at least 0.5 meter, at least 0.8 meter at least 1 meter or more. In some embodiments, the distance D2 of the electrical contact point of the second plant 500-2 from DC power source 10 (e.g., grounding electrode 30) may at least 1 meter. In some embodiments, the distances can be larger than 5 meters, 10 meters or more. In some embodiments, plant 500-2 is not the neighboring plant to plant 500-1.

According to one embodiment, the stationary plant systems 1-S comprises a plurality of plant electrodes 20 (e.g., 20-1, 20-2 and 20-3) configured to electrically and mechanically connect to a plurality of plant contact points 592 on a plant 500, namely on the same plant 500, as illustrated in FIG. 5A. According to another embodiment, at least some of the plant contact points 592 are electrically connected in parallel. According to yet another embodiment, at least some of the plant contact points 592 are electrically connected in series. According to still another embodiment, some of the plant contact points are at substantially the same height on a trunk 550 of the plant 500. According to a further embodiment, some of the plant contact points 592 are at different locations along the height of the trunk 5501.

According to one embodiment, the electrical potential of the crown of a tree may be varied due to IR drop. IR drop is the electrical potential difference between the two ends of a conducting phase during a current flow. This voltage drop across any resistance is the product of current (I) passing through resistance and resistance value (R). The IR drop is a function the electrical resistance of the electrically conductive cable 40, for example a cable 40, and the magnitude of the electrical current. The electrical resistance of the electrically conductive cable 40, for example of a cable 40, is a function of the material from which the cable 40 is made, a diameter of the cable 40, a length of the cable 40, which can be considered as the distance from the power source, and the like.

Therefore, in some embodiments, cable 40-O connecting power supply 10 to first plant electrode 20-1 may have a first thickness, first length, and/or first conductivity, cable 40-1 connecting first plant electrode 20-1 to second plant electrode 20-2 may have a second thickness, second length, and/or second conductivity, and cable 40-2 connecting second plant electrode 20-2 to third plant electrode 20-3 may have a third thickness, third length, and/or third conductivity. The thickness, second length, and/or second conductivity of each cable is set to minimize the resistivity and voltage drop on the cables. The longer the distance from the power source the thinner the cable can be.

In some embodiments, electrically conductive cables 40-O, 40-1 and or 40-2, may be bare cables, insulated cables and/or coated with an isolating material (e.g., a polymer).

According to one embodiment, the length of the cable 40, and the number and density of the plant contact points 592, influence the required diameter of the cable, and the type of internal wires of the cable.

High voltage systems include low current cables 40 with thin conductive wires, also known as strands. The electrical resistance of thin conductive wires is typically high. In addition, high voltage cables are very expensive because they include a high voltage insulation material of high quality.

Low voltage systems include high current cables that conduct high current values, and they include cables with thicker conductive wires. In addition, they contain a low-level insulation, or no insulation at all, namely the cable is bare, not coated with an insulating material.

According to one embodiment, the electrically conductive cable 40 is bare, namely not coated. According to another embodiment, the electrically conductive cable 40 is covered with an electrical insulation.

According to one embodiment, the grounding electrode 30 is configured to be electrically and mechanically connected to the growth medium 800, for example to soil 800. According to another embodiment, the grounding electrode 30 is made of an electrically conductive material. According to yet another embodiment, the grounding electrode is made of metal, for example iron, stainless steel and the like. According to still another embodiment, the grounding electrode can be a water pipe, or a conductive pole, for example a conductive fence pole, that is stuck, or inserted, in the growth medium 800.

The grounding electrode 30 can have a shape of a rod or a pole; or have a shape of a plate; or have a shape of a grid, or have any other shape that allow it to electrically and mechanically connect to the growth medium 800.

According to one embodiment, the grounding electrode 30 is an existing electrical ground, for example an electrical ground of a building, a ground of an electrical circuit, and the like.

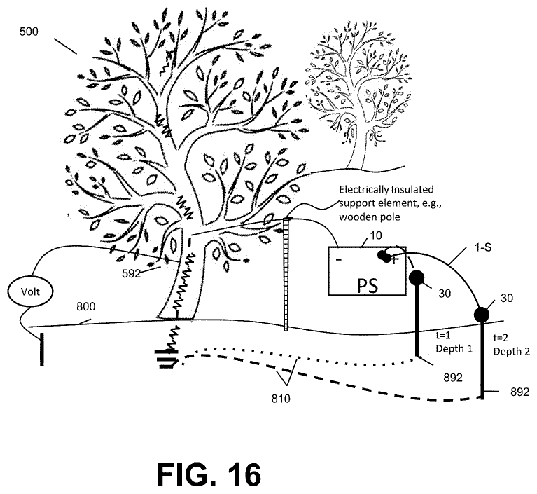

Referring now to FIG. 16, schematically illustrating some embodiments of a depth of a grounding electrode in a growth medium. According to one embodiment, the electrical potential of the crown of the tree 500 is a function of an ability of the grounding electrode 30 to form a good electrical ground. The electrical ground is affected by the depth of the grounding electrode 30 in the growth medium 800, like soil 800, the humidity of the soil 800, the presence of ice, type of soil, content of minerals in the soil, acidity of the soil, presence of fertilizers in the soil, and the like. FIG. 16 also shows an embodiment of a plurality of grounding electrodes 30 electrically connected to the power supply 10, and optionally, at different depths in the growth medium 800, for example soil 800. Additionally shown in FIG. 16 is how the electrical potential is measured with respect to the growth medium 800. In the example shown in FIG. 16, the potential of the trunk, above the midpoint and below the first division line, is monitored with respect to the growth medium 800. Similar measurements can be taken along the trunk's length, at branches, twigs, leaves and flower parts of the plant 500.

According to one embodiment, the electrical potential of the plant 500, for example of the crown of a tree 500, in the plant contact point 592 to which the plant electrode 20 is electrically and mechanically connected, is a function of the depth of the grounding electrode 30 in the soil 800. According to one embodiment, the depth of the grounding electrode 30 in the soil 800 gives rise to an electrical potential at the plant contact point 592 that is at least 90% of the voltage of the power source 10. According to another embodiment, the electrical potential at the plant contact point 592 can be brought to a level of at least 90% of the voltage of the power source 10 by apply an aquas liquid, for example water, to the soil 800 in the vicinity of the tree 500 and the grounding electrode 30.

According to one embodiment, the grounding electrode 30 is inserted into the soil 800 to a depth that gives rise to a stable electrical potential at the plant contact point 592, namely the electrical potential at the plant contact point 592 does not change when the grounding electrode 30 is inserted to higher depths in the soil 800, or when the water content of the soil 800 changes.

Additional embodiments of the operation of the combined system 12: Wisps of pollen are exhaled from the artificial pollinator barrels. Close to the barrel exit, the wisps of pollen are denser. Further away, they expand and become less dense. The pollen wisp hovers in the air, and within the tree branches for many minutes. In an embodiment, for more than 0.5, 1.0, 2.0, 5, 10, 15, 20, 30, 45, 60, 75, 90, 180, 360, 540, 720, min.

The pollen wisp continues to hover in the air and within the tree branches also after the artificial pollinator has passed. It can be described as a settling-cloud. As it hovers, it expands, and the wisp center-of-mass gets lower and lower and closer to the ground.

According to one embodiment, the polarity of the artificially distributed pollen in the settling pollen cloud is opposite the natural potential of the plant. According to another embodiment, the polarity of the artificially distributed pollen in the settling pollen cloud is positive. According to yet another embodiment, the polarity of the artificially distributed pollen is positive. According to still another embodiment, the polarity of the artificially distributed pollen in the settling pollen cloud is negative. According to a further embodiment, the polarity of the artificially distributed pollen is negative.

According to one embodiment, the stationary plant system 1-S is powered and the electrical potential of the tree crown is at a desired value and polarity. In this state, the tree crown can attract pollen from the hovering wisp. According to one embodiment, the stationary plant system 1-S is powered and the electrical potential of the tree crown is at a desired value and polarity. In this state, the tree crown can repel particles from the hovering wisp.

In an embodiment, the stationary system is powered for a duration that is less than 1, 2, 4, 7, 10, 14, 21, 28, 45, 90, 180, 270, 365 days. In an embodiment, the stationary system is powered continuously for a duration that is less than 1, 2, 4, 7, 10, 14, 21, 28, 45, 90, 180, 270, 365 days. In an embodiment, the stationary system is powered with a battery PS, whose power declines over time. In an embodiment, the stationary system is powered for a duration of the natural pollination season. In an embodiment, the stationary system is powered for a duration of the bee pollination period.

In an embodiment, the stationary system is powered for a duration that is less than 1, 2, 4, 7, 10, 14, 21 days before and or after the wind-borne pollination period. In an embodiment, the stationary system is powered for a duration that is less than 1, 2, 4, 7, 10, 14, 21 days before and or after bee pollination period.

In an embodiment, the stationary system is powered intermittently. In an embodiment, the stationary system is powered intermittently wherein the ON cycle is less than 0.5, 1, 2.5, 5, 10, 20, 30, 60, 120, 360, 540 min and the OFF cycle is less than 1, 5, 10, 20, 30, 60, 120, 360, 540, 720 min. In an embodiment, the stationary system is powered intermittently such that pollen particles overcome friction forces in the air. In an embodiment, the stationary system is powered during daylight or at night. In an embodiment, the stationary system is powered when the temperature is above −20, −10, 0, 10, 15, 20, 30, 40, 50 degrees Celsius. In an embodiment, the stationary system is not powered when the temperature is below 0, 10, 15, 20, 30, 40, 50 degrees Celsius.

In an embodiment, the artificial pollinator exhales wisps of pollen intermittently. In an embodiment, the duration of gap between each exhale cycle is less than 0.1, 0.3, 0.5, 0.75, 1, 2, 3, 5, 7, 10, 15, 25 multiples of the duration of an exhale cycle. For example, an exhale duration of 15 sec. and a gap duration of 15 sec. The intermittent cycle can vary, such that an exhale duration of 15 sec. is followed by gap duration of 15 sec, which is followed by an exhale duration of 30 sec. is followed by gap duration of 60 sec.

Following are some embodiments of a control unit of the combined system 12. It should be noted that the control unit is relevant to the stationary plant system 1-S, as shown in FIG. 15. As well as to the combined system 12. The control unit is configured to control an operation of the combined system 12, to indicate or measure various parameters, and to communicate with components of the combined system 12. The control unit may comprise a processing unit, a monitoring unit and sensors. The control unit may be configured to operate an open and closed loop control of the power source 10, or partially closed loop control, e.g., manually or periodically monitoring and controlling. The control unit may comprise a communication module capable of wired and wireless communication. According to some embodiments, the control unit comprises a controller system based on a light detector, temperature or humidity detector.

The control unit according to some embodiments, may comprise monitor configured to monitor particles from afar, for example by a drone. The control unit may comprise various types of monitors configured to monitor ambient temperature; ambient humidity; wind conditions; density of pollen in air; motion, for example direction and velocity of a pollen cloud; electrical parameters like voltage, current, resistance in the combination system, and any combination thereof. According to some embodiments, the control unit may be configured to control the plant 500, or tree 500, electrical potential at plant contact points on the plant.

Referring now to the mobile plant system. The present subject matter further provides a mobile plant system for manipulating an electrical potential of at least one plant at a time by forming an electrical circuit that includes the at least one plant. According to one embodiment, the mobile plant system comprising: at least one mobile power source configured to electrically and mechanically connect to at least one plant contact point in at least one inner tissue of at least one plant at a time, and to at least one growth medium contact point that is at a growth medium that is in contact with the at least one plant, the electrical and mechanical connection enables conduction of an electrical current between the growth medium and the at least one plant; and a mobile carrier configured to move and carry components of the mobile plant system, wherein the at least one mobile power source is electrically connected to at least one mobile plant electrode configured to electrically and mechanically connect to the at least one plant contact point, wherein the at least one mobile power source is further electrically connected to at least one mobile grounding electrode configured to electrically and mechanically connect to the at least one growth medium contact point, wherein the mobile plant system is configured to enable formation of an electrical circuit between the at least one mobile power source, the at least one plant and the growth medium, and wherein the electrical potential of at least part of the at least one plant is affected by inducing an electrical current in the electrical circuit.

According to one embodiment, the growth medium is a growth medium in which the at least one plant is planted, and the mobile carrier is configured to move on, or through, the growth medium. According to another embodiment, the growth medium is an electrically conductive liquid. According to yet another embodiment, the mobile carrier is configured to fly, or hover.

According to a further embodiment, the mobile plant module further comprising: at least one growth medium electrification station, comprising at least one growth medium connector electrically connected to an at least one growth medium contact point; and at least one growth medium station attaching element electrically connected to the at least one mobile power source, and is configured to electrically connect to the at least one growth medium connector and allow conduction of an electrical current from the at least one mobile power source to the at least one mobile contact point.

According to yet a further embodiment, the at least one inner tissue of the plant is below an epidermis layer of the plant. According to still a further embodiment, the plant is a woody plant comprising a bark, and the at least one inner tissue is below the bark. According to an additional embodiment, the electrical potential of a part of the at least one plant is affected by inducing an electrical current in the electrical circuit.

According to yet an additional embodiment, an anode of the mobile power source is electrically connected to the mobile plant electrode that is configured to electrically and mechanically connect to at least one plant contact point in at least one plant, and a cathode of the mobile power source is electrically connected to the mobile grounding electrode that is configured to electrically and mechanically connect to at least one growth medium contact point in the growth medium, causing the at least one plant to be negatively charged.

According to still an additional embodiment, a cathode of the mobile power source is electrically connected to the mobile plant electrode that is configured to electrically and mechanically connect to at least one plant contact point, and an anode of the mobile power source is electrically connected to the mobile grounding electrode that is configured to electrically and mechanically connect to at least one growth medium contact point in the growth medium, causing the at least one plant to be positively charged. According to another embodiment, the mobile power source is a mobile direct current (DC) power source. According to yet another embodiment, the mobile power source is a mobile alternating current (AC) power source. According to still another embodiment, the mobile power source is configured to supply an AC current carried on a DC current.

According to an additional embodiment, the electrical circuit comprising: a closed sub-circuit in which there is a flow of an electrical current through a part of the at least one plant and through components of the plant module; and an open sub-circuit, at another part of the at least one plant in which there is no flow of electrical current, and there is formation of an electrical potential. According to yet an additional embodiment, manipulation of the electrical current that flows through the closed sub-circuit affects the electrical potential in the open sub-circuit.

According to still an additional embodiment, the mobile plant system is configured to monitor and control the electrical potential of the at least one plant, or the electrical current that is conducted through the at least one plant, or contact of particles with part of the at least one plant, or any combination thereof.

The present subject matter also provides a method for manipulating an electrical potential of at least one plant at a time, the method comprising: providing a mobile plant system as described herein; electrically and mechanically connecting at least one mobile plant electrode of the mobile plant system to at least one plant contact point in at least one inner tissue of the at least one plant at a time; and electrically and mechanically connecting at least one mobile grounding electrode of the mobile power system to at least one growth medium contact point in a growth medium that is in contact with the at least one plant, conducting an electrical current between the growth medium and the at least one plant, thereby forming an electrical circuit that includes the mobile power source, the at least one plant and the growth medium, wherein the electrical potential of at least part of the at least one plant, is affected by the conduction of the electrical current in the electrical circuit.

According to one embodiment, the electrically connecting the at least one mobile power source to the at least one plant contact point is for a duration that is less than substantially 1 hour.

The present subject matter additionally provides a mobile plant system, as described herein, for affecting movement of electrically charged particles to, from, or within, at least one plant.

Regarding the description of the following drawings, illustrating the mobile plant system 1-M, the embodiments of the terms plant electrode 20, the plant contact point 592, the grounding electrode 30 and the growth medium contact point 892, that are described above, are relevant also to the embodiments of the mobile plant system 1-M described hereinafter, even though there is no direct reference to the aforementioned terms. In addition, the mobile plant system 1-M may be occasionally termed “mobile plant module 1-M”.

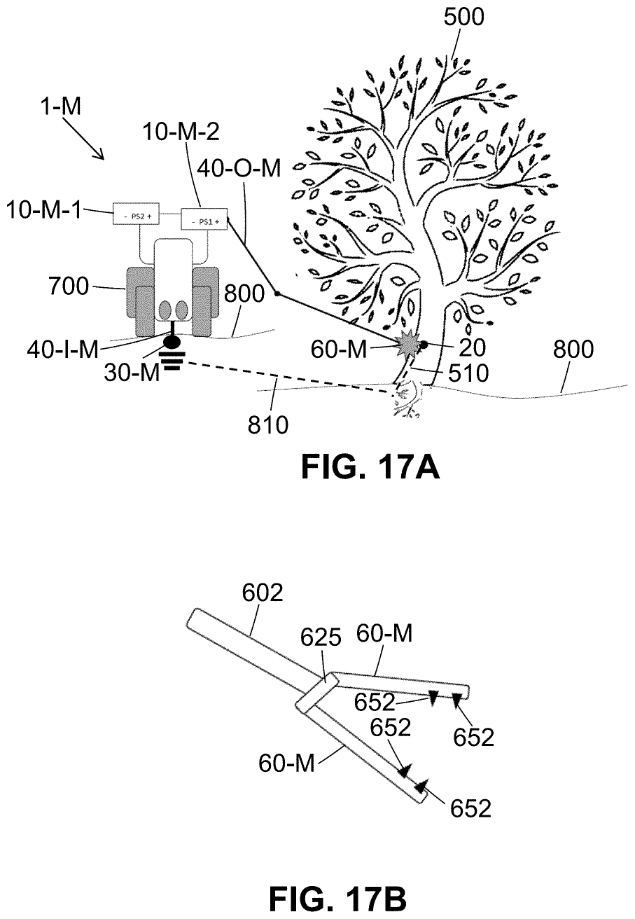

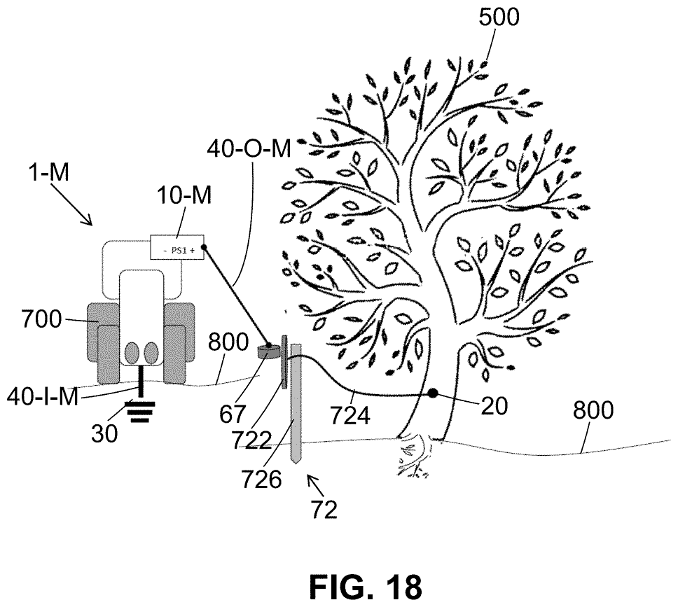

Referring now to FIG. 17A, schematically illustrating, according to some embodiments, a side view of a mobile plant module, comprising a power source electrically connected to a plant and to a growth medium. As shown in FIG. 17A, the present subject matter provides a mobile plant module. The plant module 1 that is mobile is occasionally designated hereinafter “mobile plant module 1-M”. According to another embodiment, the at least one power source 10 of the mobile plant module 1-M is mobile. The at least one power source 10 that is mobile is occasionally designated hereinafter at least one “mobile power source 10-M”. According to yet another embodiment, the at least one mobile power source 10-M, and additional components of the mobile plant module 1-M, are carried by at least one mobile carrier 700. In other words, the mobile carrier 700 is configured to move and carry components of the mobile plant module 1-M. According to one embodiment, the mobile carrier 700 is configured to move on the growth medium 800. According to another embodiment, the mobile carrier 700 is configured to move on the growth medium 800 in which the at least one plant 500 is planted. According to yet another embodiment, the mobile carrier 700 is configured to move on a ground aside the growth medium 800 in which the at least one plant 500 is planted, for example on a paved road aside soil 800 in which the at least one plant is planted. Any type of mobile carrier 700 that is configured to carry components of the mobile plant module 1-M, including the mobile power source 10-M, and move, is under the scope of the present subject matter. Some mobile carriers 700 include: a vehicle, for example a truck; an agricultural vehicle, for example a tractor; a towed vehicle, for example a towed platform, a cart, a carriage, or a wagon; an autonomous vehicle, for example an autonomous driving tractor; a robotics platform, and the like. According to another embodiment, the mobile carrier 700 is configured to move on a liquid, for example on water, for example on the water of a flooded rice field, or on an aqueous solution of hydroponic plants, and the like. According to yet another embodiment, the mobile carrier 700 is configured to move in a liquid, for example in an aqueous solution of hydroponic plants. According to a further embodiment, the liquid is electrically conductive, for example water, an aqueous solution of salts, and the like. Even though only one mobile carrier 700 is illustrated in FIGS. 17-18, it should be noted that any number of mobile carriers 700 is under the scope of the present subject matter. For example, an embodiment according to which the mobile plant module 1-M comprises two mobile carriers 700, for example a platform towed by a tractor, when some components of the mobile plant module 1-M are carried on the tractor, and other components of the mobile plant module 1-M are carried on the platform.

In other words, the present subject matter provides a mobile plant module for manipulating an electrical potential of at least one plant 500, the mobile plant module 1-M comprising: at least one mobile power source 10-M electrically connected to at least one first contact point 20 and at least one second contact point 30, in a manner that forms an electrical circuit; and at least one mobile carrier 700 configured to carry components of the mobile plant module 1-M, wherein the at least one first contact point 20 is at the at least one plant 500, wherein the at least one second contact point 30 is at the at least one plant 500, or external to the at least one plant 500, or a combination thereof, and wherein the electrical potential of the at least one plant 500, or at least one part of the at least one plant 500, is affected by inducing an electrical current in the electrical circuit.

The present subject matter further provides a mobile plant module 1-M for affecting movement of electrically charged particles to, from, or within, at least one plant, the mobile plant module 1-M comprising: at least one mobile power source 10-M electrically connected to at least one first contact point 20 and at least one second contact point 30, in a manner that forms an electrical circuit; and at least one mobile carrier 700 configured to carry components of the mobile plant module 1-M, wherein the at least one first contact point is at the at least one plant, wherein the at least one second contact point is at the at least one plant, or external to the at least one plant, or a combination thereof, and wherein the electrical potential of the at least one plant, or the at least one part of the at least one plant, is affected by inducing an electrical current in the electrical circuit.

According to the embodiment shown in FIG. 17A, the mobile plant module 1-M comprises two mobile power sources 10—a first mobile power source 10-M-1 carried by mobile carrier 700-F-1, and a second mobile power source 10-M-2 carried by mobile carrier 700-F-2, both carried on a mobile carrier 700. It should be noted that the embodiments shown in FIG. 17A, regarding the number of mobile power sources 10-M is given as an example only and should not be considered as limiting the scope of the present subject matter. Any number of mobile power sources 10-M is under the scope of the present subject matter.

According to one embodiment, the mobile power source 10-M is electrically connected to the first contact point 20 at the plant 500 with a mobile outward electrically conductive cable 40-O-M. The mobile outward electrically conductive cable 40-O-M is functionally similar to the outward electrically conductive cable 40-O described above, with respect to the electrical circuit, except that it is mobile.

According to one embodiment, the mobile plant module 1-M further comprises at least one mobile plant attaching element, that is configured to attach to a mobile outward electrically conductive cable 40-O-M, and to a plant 500, and conduct an electrical current from the mobile outward electrically conducting element 40-O-M to the plant 500. According to one embodiment, the at least one mobile plant attaching element 60-M is a mobile plant surface attaching element, that is similar to the plant surface attaching element 63 described above. According to another embodiment, the at least one mobile plant attaching element 60-M is a mobile plant penetrating attaching element, that is similar to the plant penetrating attaching element 65 described above. Since FIG. 17A, and some of the following drawings, are a general presentation of the mobile plant module 1-M, both optional embodiments—the mobile plant surface attaching element and the mobile plant penetrating attaching element, are designated hereinafter as “mobile plant attaching element 60-M”.

Referring now to FIG. 17B schematically illustrating, according to an embodiment, mobile plant attaching elements attach to an arm through an arm connector. FIG. 17B illustrates an embodiment according to which two mobile plants attaching elements 60-M are attached to the arm 602 through an arm connector 625. It should be noted though that FIG. 17B should not be considered as limiting the scope of the present subject matter in relation to the number of mobile plant attaching element 60-M that are attached to the arm 602. Any number of mobile plant attaching elements 60-M can attach to the arm 602.

Another embodiment shown in FIG. 17B relates to the number of affixing elements 652 attached to the mobile plant attaching element 60-M. According to one embodiment, at least one affixing element 652 is attached to the mobile plant attaching element 60-M. For example, FIG. 17B shows two affixing elements 652 attached to the mobile plant attaching element 60-M.

Yet another embodiment shown in FIG. 17B relates to the position and orientation of the affixing elements 652 that are attached to the mobile plant attaching element 60-M. According to one embodiment, the at least one affixing element 652 is attached to any position on the mobile plant attaching element 60-M. According to another embodiment, the at least one affixing element 652 is attached to the mobile plant attaching element 60-M in any orientation relative to the mobile plant attaching element 60-M. For example, FIG. 17B shows affixing elements 652 that are attached to a surface of the mobile plant attaching element 60-M and are substantially perpendicular to the length of the mobile plant attaching element 60-M. It should be noted, though, that FIG. 17B should not be considered as limiting the scope of the present subject matter in relation to the position and orientation of the affixing elements 652 that are attached to the mobile plant attaching element 60-M. Any position and orientation of the at least one affixing element 652 relative to the mobile plant attaching element 60-M is under the scope of the present subject matter. Any type of affixing element 652 may be attached to the mobile plant attaching element 60-M.

An embodiment of a mobile plant attaching element 60-M having sharp elements, like sharp tips, that are configured to be imbedded in a surface of a plant 500, and detach from the surface of the plant 500, is shown in FIG. 17A. According to this embodiment, the mobile plant attaching element 60-M has a body, for example a body having a substantially flat structure—for example a flat disk-like structure, or a ball-like structure, and sharp tips that extend from a surface of the body.

Returning now to FIG. 17A. According to one embodiment, the second mobile contact point 30-M is configured to be in contact with the growth medium 800 during the movement of the mobile plant module 1-M, or more particularly, during the movement of the mobile carrier 700. Any mechanism that allows the second mobile contact point 30-M to be in contact with the growth medium 800 during the movement of the mobile plant module 1-M is under the scope of the present subject matter.

According to one embodiment, the second mobile contact point 30-M is configured to be dragged by the mobile carrier 700, and simultaneously be embedded in the growth medium 800, during the movement of the mobile carrier 700. For example, the second mobile contact point 30-M and the mobile inward electrically conductive cable 40-I-M to which the second mobile contact point 30-M is connected, can have a structure and function of a plow that is dragged in the growth medium 800 during the movement of the mobile carrier 700.

According to another embodiment, the second mobile contact point 30-M is configured to be stationarily embedded in the growth medium 800, while the mobile inward electrically conductive cable 40-I-M is configured to change its length during the movement of the mobile carrier 700. For example, the mobile inward electrically conductive cable 40-I-M is a wire the is rolled over a drum carried by the mobile carrier 700, and as the mobile carrier 700 moves, the wire-shaped mobile inward electrically conductive cable 40-I-M is rolled out of the drum, thus allowing constant electrical connection between the second mobile contact point 30-M that is stationarily embedded in the growth medium 800, and the at least one mobile power source 10-M that moves as the mobile carrier 700 moves.

According to yet another embodiment, the second mobile contact point 30-M is configured to be stationarily embedded in the growth medium 800, as described above, and the mobile inward electrically conductive cable 40-I-M is elastic, thus allowing stretching of the mobile inward electrically conductive cable 40-I-M as it moves away from the second mobile contact point 30-M that is stationarily embedded in the growth medium 800.

The aforementioned embodiments of the mobile outward electrically conductive cable 40-O-M, the mobile plant attaching element 60-M, the second mobile contact point 30-M, and the mobile inward electrically conductive cable 40-I-M, allow formation of an electrical circuit that runs from the at least one mobile power source 10-M, through mobile outward electrically conductive cable 40-O-M, the plant 810, the growth medium 810, and back to the at least one mobile power source 10-M through the mobile inward electrically conductive cable 40-I-M, during movement of the mobile plant module 1-M.

Referring now to FIG. 18, schematically illustrating, according to an embodiment, a side view of a plant electrification station. According to one embodiment, illustrated in FIG. 18, the stationary plant module 1, and the mobile plant module 1-M, further comprise a plant electrification station 72. Even though FIG. 18 shows a mobile plant module 1-M comprising a plant electrification station 72, it should not be considered as limiting the scope of the present subject matter. The stationary plant module 1 can comprise a plant electrification station 72 as well. The purpose of the plant electrification station 72 is to allow indirect connection of at least one plant 500 to the stationary plant module 1, or to the mobile plant module 1-M, for conduction of an electrical current through the plant 500, without direct contact of components of the stationary plant module 1, or of the mobile plant module 1-M, with the at least one plant 500. This embodiment is important, for example, when conditions like topography, shape of plant 500, and the like, do not allow direct connection of components of the stationary plant module 1, or of the mobile plant module 1-M, with the at least one plant 500.

According to one embodiment, the plant electrification station 72 comprises a plant connector 722 electrically connected to a first contact point 20 of at least one plant 500. According to another embodiment, the plant connector 722 is electrically connected to the first contact point 20 of the at least one plant 500 with at least one plant station conducting element 724. Any type of plant station conducting element 724 is under the scope of the present subject matter, for example an electrical wire, an electricity conducting rod, and the like.

According to one embodiment, the plant electrification station 72 further comprises a plant stand 726 configured to hold the plant connector 722 in place, preferably at the vicinity of the at least one plant 500. Any type of plant stand 726 is under the scope of the present subject matter. For example, the plant stand 726 is a column that is configured to be imbedded in the growth medium 800, preferably at the vicinity of the at least one plant 500, as shown in FIG. 18. Another plant stand 726 is a rod, for example, that is configured to be imbedded in the plant 500, for example to a trunk of a tree 500. It should be noted, though, that the plant base 726 has no function in the formation of the electrical circuit that includes the at least one plant 500, and in some embodiment, the growth medium 800 as well. Therefore, according to a further embodiment, the plant stand 726 is at least partially electrically insulated, in a manner that does not allow conduction of an electrical current from the plant connector 722 to the plant 500, or to the growth medium 800, through the plant stand 726.

Still referring to FIG. 18, the plant electrification station 72 can be in any distance from the plant 500. For example, the distance between the plant electrification station 72 and the plant 500 is substantially 0.5, 1, 2, 5, 10, 20, 30, 45, 60, 120, 180, 360, 500 meters.

According to one embodiment, the stationary plant module 1 and the mobile plant module 1-M comprises a plant station attaching element 67, instead of the plant attaching element 60, or the mobile plant attaching element 60-M, as can be seen in FIG. 18. The plant station attaching element 67 is configured to electrically connect to the plant connector 722 and allow conduction of an electrical current from the power source 10, or the mobile power source 10-M, to the at least one plant 500. Any mechanism of connection of the plant station attaching element 67 with the plant connector 722 is under the scope of the present subject matter. For example, the plant station attaching element 67 can adhere to the plant connector 722, either constantly, for example in the stationary plant module 1, or transiently, for example in the stationary plant module 1 and the mobile plant module 1-M. Any mechanism of adherence is under the scope of the present subject matter, for example by gluing, welding, screwing, affixing, magnetic attraction, and the like.

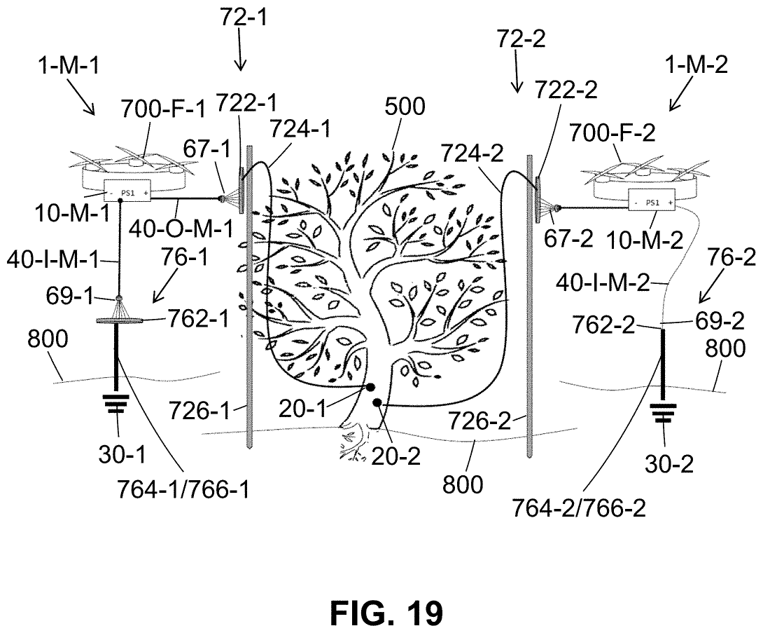

Referring now to FIG. 19, schematically illustrating, according to some embodiments, a side view of two mobile plant module for manipulating an electrical potential of at least one plant, each mobile plant module comprising a flying mobile carrier. FIG. 19 shows two mobile plant modules—a first mobile plant module 1-M-1 and a second mobile plant module 1-M-2, electrically connected to a plant 500. The first mobile plant module 1-M-1 comprises a first flying mobile carrier 700-1 carrying a first mobile power source 10-M-1, a first plant electrification station 72-1 and a first growth medium electrification station 76-1, according to embodiments described above. A first mobile outward electrically conductive cable 40-O-M-1 is electrically connected to the first mobile power source 10-M-1. A first plant station attaching element 67-1 is electrically connected to the first mobile outward electrically conductive cable 40-O-M-1, and is configured to electrically connect to a first plant connector 722-1, which is electrically connected to a first plant station conducting element 724-1, and the first plant station conducting element 724-1 is electrically connected to a first contact point 20-1 at the plant 500. In addition, the first plant connector 722-1 is held by a first plant stand 726-1. Furthermore, a first mobile outward electrically conductive cable 40-O-M-1 is electrically connected to the first mobile power source 10-M-1 at one side, while an opposite side of the first mobile power source 10-M-1 is electrically connected to a first growth medium station attaching element 69-1, via an electrically conductive cable 40-I-M-1. In some embodiments, station attaching element 69-1 is configured to electrically connect to a first growth medium connector 762-1. The first growth medium connector 762-1 is held by a first growth medium stand 766-1, which can serve also as a first growth medium station conducting element 764-1 that is configured to electrically connect to a first second contact point 30-1 at the growth medium 800.

Similarly, the second mobile plant module 1-M-2 comprises a second flying mobile carrier 700-2 carrying a second mobile power source 10-M-2, a second plant electrification station 72-2 and a second growth medium electrification station 76-2, according to embodiments described above. A second mobile outward electrically conductive cable 40-O-M-2 is electrically connected to the second mobile power source 10-M-2. A second plant station attaching element 67-2 is electrically connected to the second mobile outward electrically conductive cable 40-O-M-2, and is configured to electrically connect to a second plant connector 722-2, which is electrically connected to a second plant station conducting element 724-2, and the second plant station conducting element 724-2 is electrically connected to a second first contact point 20-2 at the plant 500. In addition, the second plant connector 722-2 is held by a second plant stand 726-2. Furthermore, a second mobile outward electrically conductive cable 40-O-M-2 is electrically connected to the second mobile power source 10-M-2 at one side, while an opposite side of the second mobile power source 10-M-2 is electrically connected to a second growth medium station attaching element 69-2, via an electrically conductive cable 40-I-M-2. In some embodiments, station attaching element 69-2 is configured to electrically connect to a second growth medium connector 762-2. The second growth medium connector 762-2 is held by a second growth medium stand 766-2, which can serve also as a second growth medium station conducting element 764-2 that is configured to electrically connect to a second contact point 30-2 at the growth medium 800.

According to one embodiment, at least two mobile plant modules 1-M are used for manipulating an electrical potential of at least one plant 500. For example, a first mobile plant module 1-M comprising a flying mobile carrier 700-F and a second mobile 1-M comprising a mobile carrier 700 traversing the growth medium 800.

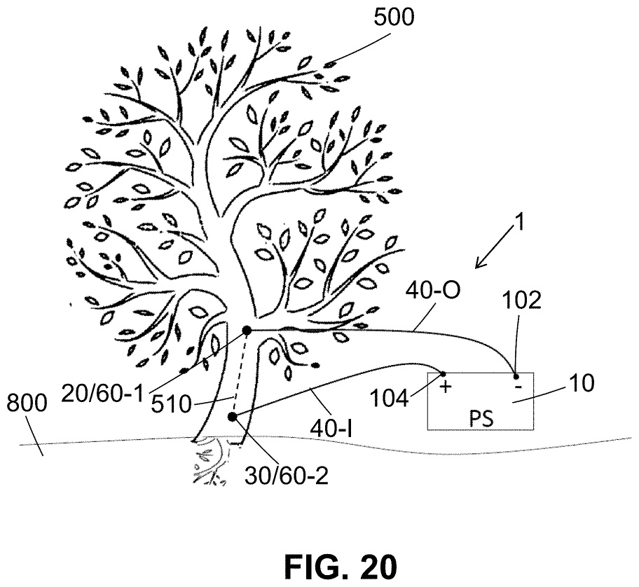

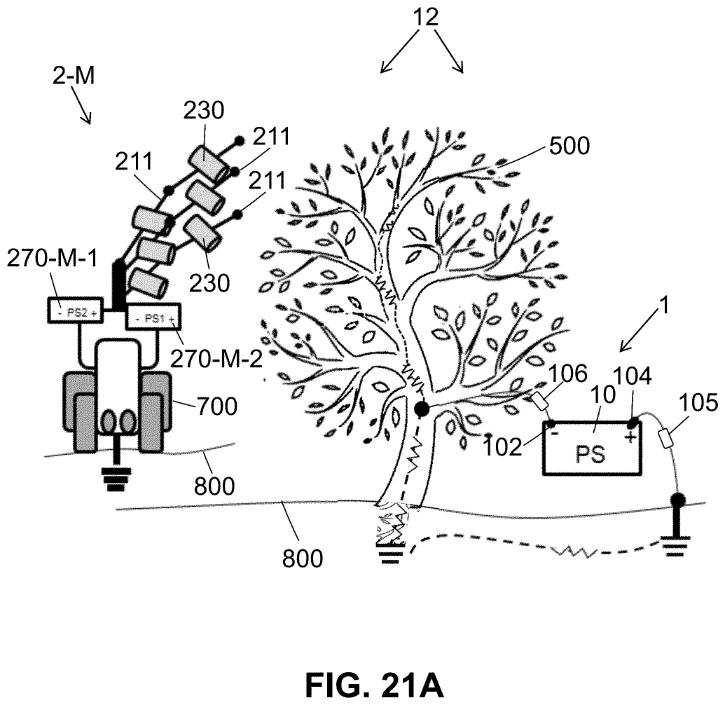

Referring now to FIG. 20 schematically illustrating, according to an embodiment, a side view a stationary plant module for manipulating an electrical potential of at least one plant, comprising a power source electrically connected to a plant at two contact points. According to the embodiment shown in FIG. 20, the second contact point 30 is at the plant 500, instead of at the growth medium 800. As a result, the closed sub-circuit does not run through the growth medium 800. Accordingly, the pathway of the closed sub-circuit shown in FIG. 20 is: an anode 102 of a power source 10—an outward electrically conductive cable 40-O—a first plant attaching element 60-1 connected to a first contact point 20 at the plant—through the plant 510—a second plant attaching element 60-2 connected to a second contact point 30 at the plant—an inward electrically conductive cable 40-I—a cathode 104 of the power source 10. It should be noted that the closed sub-circuit runs through the plant 510 between the first contact point 20 and the second contact point 30, which are both at the plant 500.

According to one embodiment, the plant power source 10 is configured to provide various levels of different characteristics of electrical power, for example various levels of electrical current, various levels of electrical voltage, and the like. Any type of mechanism, and any composition of the plant power source 10, that allows the plant power source 10 to provide the various levels of different characteristics of electrical power, is under the scope of the present subject matter. Following are some embodiments of the plant power source 10 that allow the plant power source 10 to provide the various levels of different characteristics of electrical power: According to one embodiment, the plant power source 10 comprises at least two resistors. According to another embodiment, the plant power source 10 comprises multiple capacitors. According to yet another embodiment, the plant power source 10 comprises at least two resistors and multiple capacitors. As mentioned above, the plant module 1 is configured to provide the various characteristics of the electrical power to a plant 500, or to different parts of a same plant 500, or to multiple plants 500, or any combination thereof.

Referring now to a system for manipulating an electrical potential of at least one plant and for manipulating an electrical charge of particles that interact with the at least one plant. For the sake of simplicity only, this system is occasionally referred to hereinafter as “system 12”.

Generally, the system 12 is a combination of at least one plant module 1 as described above and a particle module 2 as described above. The embodiments of the plant module 1 described above are relative to the plant module 1 of the system 12, and all the embodiments of the particle module 2 described above are relative to the particle module 2 of the system 12.

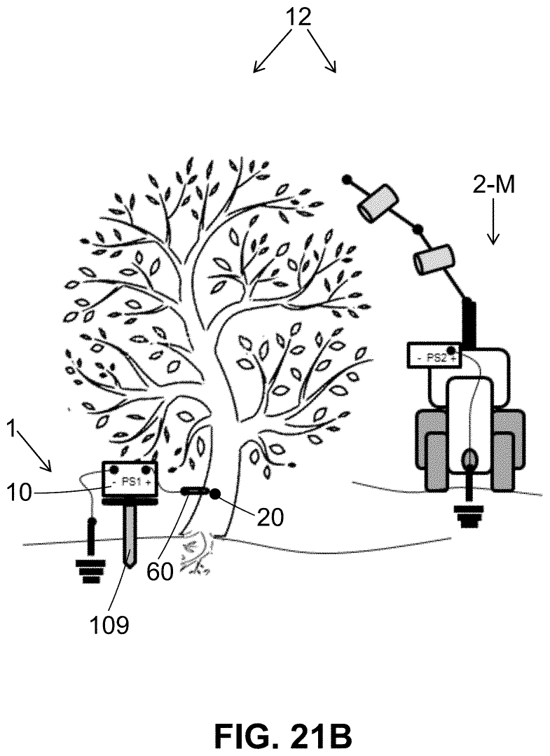

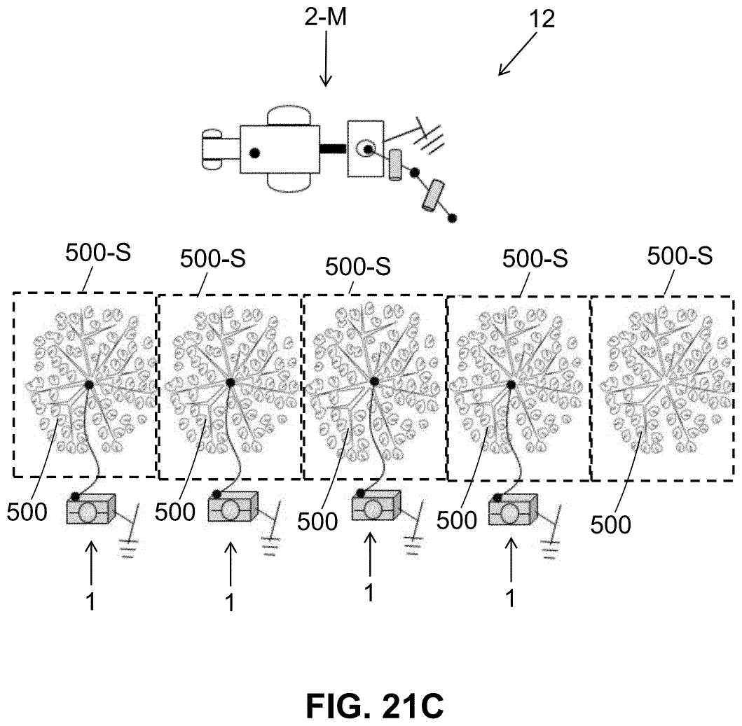

Referring now to FIGS. 21A-B schematically illustrating side views of some embodiments of a system for manipulating an electrical potential of at least one plant and for manipulating an electrical charge of particles that interact with the at least one plant. The system 12 is a combination of the plant module 1 and the particle module 2 described in detail above. Therefore, only components that are necessary for describing the various embodiments of the system 12 are marked with reference numbers in FIGS. 21A-B.

According to one embodiment, the plant module 1 of the system 12 is stationary. According to another embodiment, the plant module 1 of the system 12 is a mobile plant module 1-M. According to yet another embodiment, the particle module 2 of the system 12 is a mobile particle module 2-M. According to still another embodiment, the particle module 2 of the system 12 is stationary. Any combination of the stationary and mobile modules is under the scope of the present subject matter. According to one embodiment, when the particle module 2 is configured to manipulate the electrical charge of dispersed airborne particles, for example pollen, a mobile particle module 2-M can be used, for example when there is a need to disperse the particles in large areas such as orchards, plantations, fields, groves and the like. It should be noted that the particle modules 2 shown in FIGS. 21A-B are configured to manipulate the electrical charge of particles in a form of airborne pollen. However, this embodiment should not be considered as limiting the scope of the present subject matter. Any type of particle module 2 is under the scope of the present subject matter.

Described in other words, the system 12 comprises the plant module 1 described above, including the aforementioned embodiments of the plant module 1, and the particle module 2 described above, including the aforementioned embodiments of the particle module 2. Generally, according to one embodiment, the system 12 comprises a stationary plant module 1 and a stationary particle module 2. According to another embodiment, the system 12 comprises a stationary plant module 1 and a mobile particle module 2-M. According to yet another embodiment, the system 12 comprises a mobile plant module 1-M and a stationary particle module 2. According to still another embodiment, the system 12 comprises a mobile plant module 1-M and a mobile particle module 2-M.

FIG. 21A illustrates a system 12 comprising a stationary plant module 1 and a mobile particle module 2. Regarding the mobile particle module 2-M, FIG. 21A illustrates only some of the components of the mobile particle module 2-M, namely: at least one mobile particle power source 270-M electrically connected to at least one electrode (not seen) in at least one particle distributor 230, and further electrically connected to the soil 800. It should be noted that the components of the mobile particle module 2-M are carried by a mobile carrier 700, as shown in FIG. 21A. Regarding the at least one mobile particle power source 270-M, the mobile particle module 2-M shown in FIG. 21A comprises two mobile particle power sources 270-M: a first mobile particle power source 270-M-1 and a second mobile particle power source 270-M-2. Both mobile particle power sources 270-M are electrically connected together to the soil 800.

One feature of the mobile particle module 2-M that is shown in FIG. 21A relates to the barrel mounting pole P11. In FIGS. 21A-B, the barrel mounting pole P11 is designated “mounting pole 211”. According to one embodiment, the particle module 2, and the mobile particle module 2-M, comprises at least one mounting pole 211 that is configured to hold at least one particle distributor 230. The mobile particle module 2-M shown in FIG. 21A comprises an amount of three mounting poles 211, when each one of the three mounting poles 211 holds multiple particle distributors 230.

According to one embodiment, the system 12 shown in FIG. 21A operates on a plant 500, in a form of a tree 500. According to this particular embodiment, the system 12 is configured to control, or affect, or both control and affect, a trajectory of air-borne pollen dispersed from the mobile plant module 2-M to the tree 500. The plant module 1 charges parts of the tree 500, particularly stigmas in flowers of the tree 500, with a negative electrical charge (note that the anode 102 of the power source 10 is electrically connected to the tree 500, and the cathode 104 is electrically connected to the soil 800). In addition, the mobile particle module 2-M is configured to artificially pollinate the tree 500 with pollen, for example insect-borne pollen. For this purpose, the mobile particle module 2-M is configured to manipulate the natural electrical charge of pollen, and more specifically, to charge the pollen with a positive electrical charge, and disperse the manipulated electrically charged pollen toward the tree 500. According to this embodiment, the operation of the system 12 is a combination of charging the tree 500 with a negative electrical charge, by the stationary plant module 1, and charging of the dispersed pollen with a positive electrical charge, by the mobile particle module 2-M. This combination enhances the movement of the pollen toward stigmas of the tree 500, for example in order to increase the rate of pollination of the flowers of the tree 500, thereby increase the yield of fruits produced by the tree 500. In an embodiment, the polarity of the plant 500 and particles is reversed. That is, the plant module 1 charges parts of the plant 500, for example a tree 500, particularly stigmas in flowers of the tree 500, with a positive electrical charge, and the mobile particle module 2-M is configured to charge the pollen with a negative electrical charge, and disperse the manipulated electrically charged pollen toward the tree 500.

Still referring to FIG. 21-A, the system 12 further comprises at least one electrical current changer between the soil 800 and the plant power source 10, and between the plant power source 10 and the plant 500. For example, a first electrical current changer 105 is shown in FIG. 21A between the cathode 104 and the soil 800, and a second electrical current changes 106 is shown between the anode 102 and the plant 500. The electrical current changer 105/106 is configured to change the electrical current that flows through the electrical circuit generated by the plant module 1. Any type of electrical current changer 105/106 is under the scope of the present subject matter, for example an electrical resistor. For example, a 1G Ohm resistor that is configured to allow flow of only high current intensities, while blocking the flow of low current intensities. This embodiment, of at least one electrical current changer 105/106, allows control of the electrical current to which the at least one plant is exposed. In should be noted in this regard, that the embodiment of the at least one electrical current changer 105/106 is relevant to any type and embodiment of the plant module 1 described herein.

The system 12 shown in FIG. 21B shows a stationary or semi stationary, plant module 1. According to an embodiment, the stationary plant module 1 shown in FIG. 21B comprises a power source stand 109 configured to hold at least one power source 10.

Referring now to FIG. 21C, schematically illustrating, according to an embodiment, a top view of a system for manipulating an electrical potential of at least one plant and for manipulating an electrical charge of particles that interact with the at least one plant. FIG. 21C shows multiple plants 500, in a form of trees 500, arranged in a row. Another embodiment shown in FIG. 21C is that the multiple plants 500 are divided to subsets 500-S of plants 500, and each subset 500-S of plants 500 comprises at least one plant 500, for example a row of trees 500 in an orchard. According to one embodiment, the dashed line defining the subsets 500-S is virtual and drawn to show the concept of the subsets 500-S. According to another embodiment, the dashed line defining the subsets 500-S represents a tangible border that separates between the different subsets 500-S. In the embodiment shown in FIG. 21C, each subset 500-S of plants 500 comprises one plant 500. In addition, a stationary plant module 1 is electrically connected to each subset 500-S of plants 500, except one subset 500-S. Furthermore, a mobile particle module 2-M moves along the row of plants 500, and, for example disperses manipulated electrically charged pollen towards the plants 500 that their electrical potential is manipulated by the stationary plant module 1 electrically connected to them.

The embodiment shown in FIG. 21C is an embodiment of a way to artificially pollinate multiple plants 500 with the system 12 of the present subject matter, when the system 12 improves the efficiency of the pollination of the plants 500 due to the manipulation of the electrical potential of the plants 500 in combination with manipulation of the electrical potential of the dispersed particles, for example pollen, or a pesticide, or a fertilizer, that interact with the plants 500.

To summarize, FIGS. 21A-C illustrated various embodiments of the system 12, or combinations of various types of the plant module 1 and the particle module 2. It should be noted that the embodiments and combinations of the two modules that are shown in FIGS. 21A-C are given as an example only, and that any combination of various embodiments of the plant module 1 and the particle module 2 are under the scope of the present subject matter.

According to one embodiment, the system 12 is configured to control trajectory of manipulated electrically charged particles. According to another embodiment, the system 12 is configured to control velocity of movement of manipulated electrically charged particles. According to yet another embodiment, the system 12 is configured to control trajectory and velocity of movement of manipulated electrically charged particles. As can be understood from the description of the present subject matter, both plant module 1 and particle module 2 are configured to affect, or control, or both affect and control, the trajectory of manipulated electrically charged particles. The combined operation of the plant module 1 and the particle module 2 has a stronger effect on the trajectory of the manipulated electrically charged particles than each one alone. The coordinated operation of the plant module 1 and the particle module 2 has a stronger effect on the trajectory of the manipulated electrically charged particles than each one alone. In addition, the combined operation of the plant module 1 and the particle module 2 enables improved control of the trajectory of the manipulated electrically charged particles. In this regard, the system 12 is configured, for example, to modify motion of the manipulated electrically charged particles.

Generally, the trajectory of electrically charged particles is affected by wind and gravity as well as momentum provided to the particles by the system 12, and the electrical potential of bodies in proximity of the electrical charged particles. Thus, the system 12 of the present subject matter provides additional forces that affect attraction or repulsion of manipulated electrically charged particles to, or from, the at least one plant. In this sense, the plant module 1 and the particle module 2 create forces that affect the trajectory of the manipulated electrically charged particles. These forces originate from different sources and typically have different vectors. The force that is created by the system 12 is an integration of the forces created by the plant module 1 and the particle module 2. As a result of the forces that are applied on the particles, the particles disperse and move in a certain displacement vector.

When a particle moves from an initial position in space to a final position in space, a displacement vector of the particle can be defined as a difference between the final position and the initial position. In other words, the displacement vector is a vector whose length is the shortest distance from the initial position in space to the final position in space. The displacement vector quantifies both a distance and direction of a net, or total, motion of the particle along a straight line from the initial position in space to the final position in space. A particle motion path comprises at least one movement from an initial position in space to a final position in space.

In relation to the system 12 of the present subject matter, the initial position in space of the particle is the distributor 230, and the final position in space of the particle is a position in space that is reached by the particle. In relation to dispersing particles towards plants 500 it is desired that the final position is target position on the plant 500. However, prior art devices and mechanism sometimes fail in directing the particles to their target position on the plant 500. For example, the dispersed particles can miss the target position on the plant. Therefore, an aim of the system 12 of the present subject matter is modify the displacement vector of the particle from the distributor 230 to the target position on the plant 500, instead of from the distributor 230 to a missed position in space. Accordingly, the system 12 is indeed configured to modify the displacement vector of the particles.

For example, in case when the particles are pollen, using the system 12 can increase attraction forces toward the at least one plant that act on manipulated electrically charged pollen, resulting in more pollen grains reaching flowers of the at least one plant.

Similarly, for example, in case when the particles are pesticide droplets, using the system 12 can apply repulsion forces on manipulated electrically charged pesticide droplets from the at least one plant, resulting in control of the trajectory of the manipulated electrically charged pesticide droplets—for example preventing from the manipulated electrically charged pesticide droplets from reaching certain parts of the at least one plant, as desired. In other words, the system 1 is configured to control repulsion of manipulated electrically charged particles from the at least one plant.

According to one embodiment, when it is desired to attract particles to the at least one plant, the system 12 is configured to manipulate the electrical charge of the particles and manipulate the electrical potential of the at least one plant, to produce manipulated electrically charged particles having a polarity that is opposite the polarity of the manipulated electrical potential of the at least one plant. For example, the system 12 is configured to produce positively manipulated electrically charged particles and at least one plant having a negative manipulated electrical potential; or the system 12 is configured to produce negatively manipulated electrically charged particles and at least one plant having a positive manipulated electrical potential.

According to another embodiment, when it desired to repel particles from the at least one plant, the system 12 is configured to manipulate the electrical charge of the particles and manipulate the electrical potential of the at least one plant, to produce manipulated electrically charged particles having a polarity that is similar to the polarity of the manipulated electrical potential of the at least one plant. For example, the system 12 is configured to produce positively manipulated electrically charged particles and at least one plant having a positive manipulated electrical potential; or the system 12 is configured to produce negatively manipulated electrically charged particles and at least one plant having a negative manipulated electrical potential.

Following are some embodiments of the system 12, and a method using the system 12. The present subject matter provides a system 12 for manipulating an electrical potential of at least one plant and for manipulating an electrical charge of particles that interact with the at least one plant and disperse the particles, the system 12 comprising: at least one plant module 1 configured to manipulate the electrical potential of at least one plant 500; and at least one particle module 2 configured to manipulate a natural electrical charge of particles that interact with the at least one plant to obtain manipulated electrically charged particles and disperse the manipulated electrically charged particles.

According to an embodiment, an operation of the at least one plant module 1 is coordinated with an operation of the at least one particle module 2. According to an embodiment, the plant module 1 is either a stationary plant module 1, or a mobile plant module 1-M.

According to an embodiment, the particle module 2 is either a stationary particle module 2, or a mobile particle module 2-M. According to an embodiment, the plant module 2 comprising at least one plant power source 10. According to an embodiment, the at least one plant power source 10 comprising at least one direct current (DC) power source. According to an embodiment, the at least one plant power source 10 comprising at least one alternating current (AC) plant power source. According to an embodiment, the plant module 1 comprising multiple plant power sources 10.

According to an embodiment, the multiple plant power sources 10 are each electrically connected to a different plant 500. According to an embodiment, the multiple plant power sources 10 are each electrically connected to a different part of a same plant 500.

According to an embodiment, the multiple plant power sources 10 are configured to provide an electrical voltage, wherein a characteristic of the electrical voltage is different between at least two plant power sources 10, wherein the characteristic is voltage magnitude, or voltage polarity, or voltage frequency, or any combination thereof.

According to an embodiment, the particle module 2 comprising at least one particle power source 270. According to an embodiment, the at least one particle power source 270 comprising at least one DC power source 270. According to an embodiment, the at least one particle power source 270 comprising at least one AC plant power source 270. According to an embodiment, the particle module 2 comprising multiple particle power sources 270. According to an embodiment, the particle module 2 comprising a first particle power source 270-1 and a second particle power source 270-2.

According to an embodiment, the manipulated electrically charged particles 299 are manipulated electrically charged pollen, and the system 12 is configured to increase attraction forces toward the at least one plant 500 that act on the manipulated electrically charged pollen.

According to an embodiment, the manipulated electrically charged particles 299 are manipulated electrically charged particles, and the system 12 is configured to modify a displacement vector of the manipulated electrically charged particles.

According to an embodiment, the manipulated electrically charged particles 299 have a positive electrical charge. According to an embodiment, the manipulated electrically charged particles 299 have a negative electrical charge.

According to an embodiment, the manipulated electrically charged particles 299 are a mixture of manipulated electrically charges particles 299 having a positive electrical charge and manipulated electrically charged particles 299 having a negative electrical charge.

The present subject matter further provides a method for manipulating an electrical potential of at least one plant 500 and for manipulating a natural electrical charge of particles that interact with the at least one plant, the method comprising: providing a system 12 for manipulating an electrical potential of at least one plant 500 and for manipulating a natural electrical charge of particles that interact with the at least one plant 500, the system comprising: at least one plant module 1 configured to manipulate the electrical potential of at least one plant 200; and at least one particle module 2 configured to manipulate the natural electrical charge of particles that interact with the at least one plant 500 to obtain manipulated electrically charged particles 299 and disperse the manipulated electrically charged particles 299; manipulating the electrical potential of at least one plant 500; manipulating the natural electrical charge of particles that interact with the at least one plant 500 to obtain manipulated electrically charged particles 299; and dispersing the manipulated electrically charged particles 299 in a vicinity of the at least one plant.

According to an embodiment, an operation of the at least one plant module 1 is coordinated with an operation of the at least one particle module 2, and wherein the manipulating the electrical potential of the at least one plant 500 is coordinated with the manipulating of the natural electrical charge of the particles that interact with the at least one plant 500.

According to an embodiment, a quantity of the electrical charge of the manipulated electrically charged particles is different from a quantity of the electrical charge of the natural electrically charged particles.

According to an embodiment, an electrical polarity of the manipulated electrically charged particles 299 is opposite to an electrical polarity of the natural electrically charged particles.

According to an embodiment, an electrical polarity of the manipulated electrically charged particles 299 is identical to an electrical polarity of the natural electrically charged particles.

According to an embodiment, the particles are pollen, and the manipulated electrically charged particles 299 are manipulated electrically charged pollen. According to an embodiment, the particles are pesticide particles, and the manipulated electrically charged particles 299 are manipulated electrically charged pesticide particles.

According to an embodiment, the particles are fertilizer particles, and the manipulated electrically charged particles 299 are manipulated electrically charged fertilizer particles. In some embodiments, the fertilizer may be added to growth medium 800 and the electrical potential provided to plants 500, 500-1, 500-2, and 500-3 may accelerate the provision of the fertilizer (e.g., water soluble chemicals) to the roots of the plants.

According to an embodiment, the manipulating the electrical potential of the at least one plant 500 coincides with the dispersing of the manipulated electrically charged particles 299 in a vicinity of the at least one plant 500.

According to an embodiment, the manipulating the electrical potential of the at least one plant 500 is starting before, or after, dispersing the manipulated electrically charged particles 299 towards the at least one plant 500, and there is a start time gap between a starting of the manipulating the electrical potential of the at least one plant 500 and a starting of the dispersion of the manipulated electrically charged particles 299 towards the at least one plant 500. According to an embodiment, the start time gap is up to substantially 0.5, 1, 2, 5, 10, 20, 30, 45, 60, 90 seconds, or 2, 5, 10, 20, 30, 45, 60, 120, 180, 360, 500, 1,000 minutes.

According to an embodiment, the manipulating the electrical potential of the at least one plant 500 is ending before, or after, the dispersing the manipulated electrically charged particles 299 towards the at least one plant 500, and there is an end time gap between an ending of the dispersion of the manipulated electrically charged particles 299 towards the at least one plant 500 and an ending of the manipulating the electrical potential of the at least one plant 500. According to an embodiment, the end time gap is at least substantially 0.5, 1, 2, 5, 10, 20, 30, 45, 60, 90 seconds, or 2, 5, 10, 20, 30, 45, 60, 120, 180, 360, 500, 1,000 minutes.

According to an embodiment, the manipulating the electrical potential of the at least one plant 500 starts when the particle module 2 is at a working distance from the at least one plant 500. According to an embodiment, the working distance is up to substantially 0.1, 0.3, 0.5, 0.75, 1, 2, 3, 4, 5, 6, 7, 8, 9, 10, 20, 30, 50, 100, 200, 500, 1,000, 2,000, 5,000, or 10,000 meters.

According to an embodiment, the manipulated electrically charged particles 299 have a positive electrical charge. According to an embodiment, the manipulated electrically charged particles 299 have a negative electrical charge. According to an embodiment, the manipulated electrically charged particles 299 are a mixture of manipulated electrically charges particles 299 having a positive electrical charge and manipulated electrically charged particles 299 having a negative electrical charge.

According to one embodiment, in the system 12, an operation of the at least one plant module 1 is coordinated with an operation of the at least one particle module 2. According to another embodiment, the method further comprising: coordinating the manipulating the electrical potential of the at least one plant 500 with the manipulating of the electrical charge of the particles that interact with the at least one plant 500.

Some additional embodiments of the system 12, and the method using the system 12, are provided hereinafter. According to one embodiment, the plant module 1 comprises a first plant power source 10-1 and a second plant power source 10-2. According to one embodiment, the multiple plant power sources 10 comprise at least one DC power source 10 and at least one AC power source 10.

According to one embodiment, an electrical polarity of the manipulated electrically charged particles 299 is similar to the electrical polarity of the particles before the manipulation of their electrical charge, and the quantity of the electrical charge of the manipulated electrically charged particles 299 is different from the quantity of the electrical charge of the particles before the manipulation of their electrical charge, namely the quantity of the electrical charge of the manipulated electrically charged particles 299 is either higher, or lower, than the electrical charge of the particles before the manipulation of their electrical charge.

According to another embodiment, an electrical polarity of the manipulated electrically charged particles 299 is opposite to the electrical polarity of the particle before the manipulation of their electrical charge, and the quantity of the electrical charge of the manipulated electrically charged particles 299 is similar to the quantity of the electrical charge of the particles before the manipulation of their electrical charge. For example, when the natural particles are positively charged, the manipulated electrically charged particles 299 are negatively charged; or when the natural particles are negatively charged, the manipulated electrically charged particles 299 are positively charged.

According to yet another embodiment, an electrical polarity of the manipulated electrically charged particles 299 is opposite to the electrical polarity of the particle before the manipulation of their electrical charge, and the quantity of the electrical charge of the manipulated electrically charged particles 299 is different from the quantity of the electrical charge of the particles before the manipulation of their electrical charge.

According to a further embodiment, the manipulating the electrical potential of the at least one plant 500 is starting before dispersing the manipulated electrically charged particles 299 towards the at least one plant 500, and there is a start time gap between a starting of the manipulating the electrical potential of the at least one plant and a starting of the dispersion of the manipulated electrically charged particles towards the at least one plant 500.

According to still a further embodiment, the start time gap is at least substantially 0.5, 1, 2, 5, 10, 20, 30, 45, 60 seconds, or 2, 5, 10, 20, 30, 45, 60, 120, 180, 360, 500, 1,000 minutes.

According to an additional embodiment, the manipulating the electrical potential of the at least one plant is ending after the dispersing the manipulated electrically charged particles 299 towards the at least one plant 500. That is, there is an end time gap between an ending of the dispersion of the manipulated electrically charged particles 299 towards the at least one plant 500 and an ending of the manipulating the electrical potential of the at least one plant.

According to still an additional embodiment, the end time gap is up to substantially 0.5, 1, 2, 5, 10, 20, 30, 45, 60 seconds, or 2, 5, 10, 20, 30, 45, 60, 120, 180, 360, 500, 1,000 minutes.

According to one embodiment, the manipulating the electrical potential of the at least one plant is starting when the particle module is at a working distance from the at least one plant.

Additional embodiments in, relating to a plurality of power sources that are part of the stationary plant system: According to one embodiment, the stationary plant system 10 comprises a plurality of power sources 10. According to another embodiment, the plurality of power sources 10 are each electrically connected to a different plant 500. According to yet another embodiment, the plurality of power sources 10 are each electrically connected to a different part of a same plant 500.

According to still another embodiment, the plurality of power sources is configured to provide an electrical voltage, wherein a characteristic of the electrical voltage is different between at least two power sources 10, wherein the characteristic is voltage magnitude, or voltage polarity, or voltage frequency, or any combination thereof.

### Experimental Results

### Example I—Combining the Manipulating the Electrical Potential of Trees with Artificial Pollination

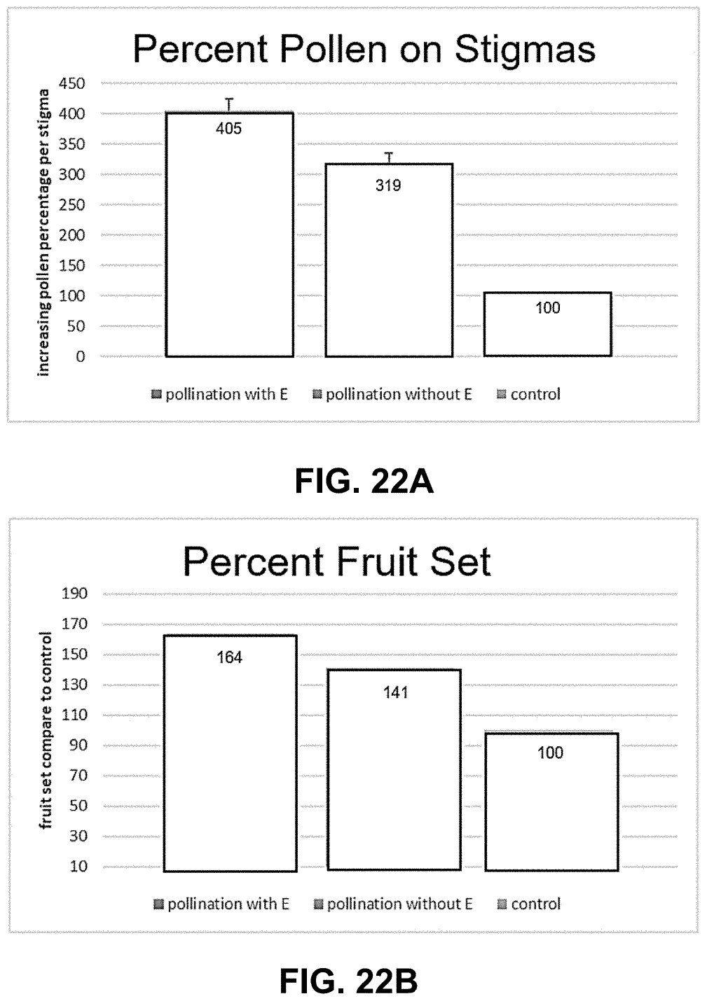

Referring now to FIGS. 22A and 22B showing results of manipulating the electrical potential of trees using system S-1 and the method of FIG. 5B. in almonds orchard during the blooming season according to some embodiments of the invention. The percentage of pollen per stigmas and the percentage of fruit production was measured for three groups of trees. In each tree four branches having substantially similar blooming (substantially similar amount of flowers) were chosen for measuring the percentage of pollen per stigmas and four different branches having substantially similar blooming were chosen for measuring the percentage of fruit production. The percentage of pollen per stigmas were measured by removing the branches, 3 days after the pollination) immersed in a fixating solution, and the number on pollens in the fixating solution was measured under a microscope. The percentage of fruit production was directly measured by comparing the fruit production of the selected branch prior to harvesting.

Group I was provided with an artificial pollination (using system such as systems 2 or 2-M) together with the provision of electrical potential, using system 1-S, to multiple pollinated trees. Group II was provided with an artificial pollination (using system such as systems 2 or 2-M) without the addition of the electrical potential. Following the artificial pollination the chosen branches were covered to avoid natural pollination. Group III was the control group. Each one of the groups included at least 5 trees. The DC power of 76/volts was provided to the trees of group I for at least 20 minutes during the activation of artificial pollination systems 2 or 2-M.

As clearly shown in FIGS. 22A and 22B adding the electrical potential during the artificial pollination process increased the pollen percentage per stigma by 20% and the fruit production by at least 10%.

### Example II—Combining the Manipulating the Electrical Potential of Trees with Natural Pollination

Various types of trees were provided with electrical potential of 12 Volts for at least the entire blooming season. Table 1 summarizes the increase in the fruit production in comparison to a control group (as disclosed in Example I).

TABLE 1

| | | Increase in fruit |
|---|---|---|
| Type | Species | production percentage |
| Prunus subg. Cerasus (Cherry) | 40E50 | 9.5% |
| Prunus subg. Cerasus (Cherry) | Royal Dawn | 11% |
| Pear | Spadona | 3% |
| Apple | Gala | 9% |
| Olive | Cornice | 6.5% |

It is appreciated that certain features of the subject matter, which are, for clarity, described in the context of separate embodiments, may also be provided in combination in a single embodiment. Conversely, various features of the subject matter, which are, for brevity, described in the context of a single embodiment, may also be provided separately or in any suitable sub combination.

Although the subject matter has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method for manipulating an electrical potential of a plurality of plants, comprising:
electrically and mechanically connecting at least a first plant electrode to a first plant of a plurality of plants;
electrically and mechanically connecting at least a second plant electrode to a second plant of the plurality of plants;
connecting the first plant electrode to the second plant electrode;
connecting the first plant electrode to a DC power source;
connecting the DC power source to a growth medium via a grounding electrode; and
continuously providing DC power to the plurality of plants,
wherein electrically and mechanically connecting the first and second plant electrode to the first and second plants comprises inserting at least a portion of each plant electrode into inner layers of the plant, in proximity to a lowest branching point of each plant.

2. The method of claim 1, wherein each plant electrode comprises a body and one or more conductive affixing elements configured to penetrate into the inner layers of the plant.

3. The method of claim 1, wherein the grounding electrode is located at least 5 meters from at least one plant electrode.

4. The method of claim 1, wherein continuously providing the DC power is for a duration of at least 2 minutes.

5. The method of claim 1, wherein connecting the first plant electrode to a DC power source is via an electrically conductive cable isolated from the growth medium.

6. The method of claim 1, wherein inserting the at least a portion of each plant electrode to each plant is at a height of at least 50% of the height of the lowest branching point from the growth medium.

7. The method of claim 1, wherein inserting the at least a portion of each plant electrode to each plant is at a height of at least 75% of the height of the lowest branching point from the growth medium.

8. The method of claim 1, wherein connecting the first plant electrode to the second plant electrode is via a first electrically conductive cable,
and wherein the method further comprising:
electrically and mechanically connecting at least a third plant electrode to a third plant of the plurality of plants; and
connecting the second plant electrode to the third plant electrode via a second electrically conductive cable;
wherein the first electrically conductive cable differs from the second electrically conductive cable by at least one of, thickness, length, and conductivity.

9. The method of claim 1, further comprising electrically connecting a plurality of plant electrodes, directly to a DC power source and to a plant electrode of a different plant.

10. The method of claim 1, wherein the plurality of plants belongs to a row of trees in an orchard.

11. The method of claim 1, wherein the growth medium is soil.

12. A system for manipulating an electrical potential of plants, comprising:
two or more plant electrodes, each electrically and mechanically connectable to a plant, wherein at least a portion of each plant electrode is insertable into inner layers of the plant;
a DC power source directly connected to a first plant electrode of a first plant;
a grounding electrode connected to the DC power source and electrically grounded to a growth medium; and
at least one electrically conductive cable, configured to connect the first plant electrode to a second plant electrode.

13. The system of claim 12, wherein each plant electrode comprises a body and one or more conductive affixing elements configured to penetrate into the inner layers of the plant.

14. The system of claim 13, wherein the conductive affixing elements have a shape selected from, a tack-like shape, or a nail-like shape, or a blade-like shape, having a sharp tip, and edge.

15. The system of claim 12, wherein the grounding electrode is located at least 5 meters from at least one plant electrode.

16. The system of claim 12, wherein the at least one electrically conductive cable is electrically isolated from the growth medium.

17. The system of claim 12, comprising:
a first electrically conductive cable for electrically connecting a first plant electrode of a first plant to a second plant electrode of a second plant; and
a second electrically conductive cable, for electrically connecting the second plant electrode of the second plant to a third plant electrode of a third plant,
wherein the first electrically conductive cable differs from the second electrically conductive cable by at least, thickness, length, and conductivity.

18. The system of claim 12, comprising a plurality of DC power sources, each being connected to the plant electrode of a first plant from a group of plants.

19. The system of claim 18, further comprising a plurality of connecting cables sequentially connecting all the plant electrodes of all other plants of the group to the first plant electrode of the first plant.
